(19) United States
(12) Patent Application Publication
(10) Pub. No.: US 2018/0127654 A1
(43) Pub. Date: May 10, 2018

(54) PIPERIDINE DERIVATIVE, LIQUID CRYSTAL COMPOSITION AND LIQUID CRYSTAL DISPLAY DEVICE

(71) Applicants: JNC CORPORATION, Tokyo (JP); JNC PETROCHEMICAL CORPORATION, Tokyo (JP)

(72) Inventors: Yasuyuki GOTOH, TOKYO (JP); Kazuo OKUMURA, CHIBA (JP)

(73) Assignees: JNC CORPORATION, Tokyo (JP); JNC PETROCHEMICAL CORPORATION, Tokyo (JP)

(21) Appl. No.: 15/805,142

(22) Filed: Nov. 7, 2017

(30) Foreign Application Priority Data
Nov. 9, 2016 (JP) 2016-218624

(51) Int. Cl. C09K 19/54; C09K 19/30; C09K 19/34; C07D 211/12; C07D 211/22; C07D 211/94; C07D 211/18

(52) U.S. Cl. CPC C09K 19/54; C09K 2019/3422; C09K 19/3003; C09K 19/3001; C09K 19/3402; C09K 19/3066; C07D 211/12; C07D 211/22; C07D 211/94; C07D 211/18; C09K 2019/3077; C09K 2019/3004; C09K 2019/3009; C09K 2019/301; C09K 2019/3016; C09K 2019/308; C09K 2019/3083; C09K 19/3068

(57) ABSTRACT

Provided are a compound having an effect on preventing photolysis of a liquid crystal composition, and high solubility in the liquid crystal composition, a liquid crystal composition containing the compound and a liquid crystal display device including the composition.

A compound is represented by formula (1), a liquid crystal composition contains the compound and a liquid crystal display device uses the composition.

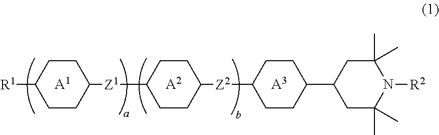

In formula (1), for example, R1 is alkyl having 1 to 10 carbons; R2 is hydrogen, hydroxy, oxy radical or alkyl; ring A1, ring A2 and ring A3 are independently 1,4-cyclohexylene, 1,4-phenylene or pyridine-2,5-diyl; Z1 and Z2 are independently a single bond or alkylene; and a and b are independently 0 or 1.

### CROSS-REFERENCE TO RELATED APPLICATIONS

This application claims the priority benefit of Japanese application serial no. 2016-218624, filed on Nov. 9, 2016. The entirety of the above-mentioned patent application is hereby incorporated by reference herein and made a part of this specification.

### TECHNICAL FIELD

The invention relates to a piperidine derivative, a liquid crystal composition and a liquid crystal display device. In particular, the invention relates to a compound having tetramethylpiperidinyl, a liquid crystal composition containing the compound and having positive or negative dielectric anisotropy, and a liquid crystal display device including the composition.

### BACKGROUND ART

In a liquid crystal display device, a classification based on an operating mode for liquid crystal molecules includes a phase change (PC) mode, a twisted nematic (TN) mode, a super twisted nematic (STN) mode, an electrically controlled birefringence (ECB) mode, an optically compensated bend (OCB) mode, an in-plane switching (IPS) mode, a vertical alignment (VA) mode, a fringe field switching (FFS) mode and a field-induced photo-reactive alignment (FPA) mode. A classification based on a driving mode in the device includes a passive matrix (PM) and an active matrix (AM). The PM is classified into static, multiplex and so forth, and the AM is classified into a thin film transistor (TFT), a metal insulator metal (MIM) and so forth. The TFT is further classified into amorphous silicon and polycrystal silicon. The latter is classified into a high temperature type and a low temperature type based on a production process. A classification based on a light source includes a reflective type utilizing natural light, a transmissive type utilizing backlight and a transflective type utilizing both the natural light and the backlight.

The liquid crystal display device includes a liquid crystal composition having a nematic phase. The composition has suitable characteristics. An AM device having good characteristics can be obtained by improving characteristics of the composition. Table 1 below summarizes a relationship in the characteristics. The characteristics of the composition will be further described based on a commercially available AM device. A temperature range of the nematic phase relates to a temperature range in which the device can be used. A preferred maximum temperature of the nematic phase is about 70° C. or higher, and a preferred minimum temperature of the nematic phase is about −10° C. or lower. Viscosity of the composition relates to a response time in the device. A short response time is preferred for displaying moving images on the device. A shorter response time even by one millisecond is desirable. Accordingly, a small viscosity in the composition is preferred. A small viscosity at low temperature is further preferred.

TABLE 1
Characteristics of composition and characteristics of AM device

| No. | Characteristics of composition | Characteristics of AM device |
|---|---|---|
| 1 | Wide temperature | Wide usable |
| | range of a nematic phase | temperature range |
| 2 | Small viscosity | Short response time |
| 3 | Suitable optical anisotropy | Large contrast ratio |
| 4 | Large positive or negative | Low threshold voltage |
| | dielectric anisotropy | and small electric |
| | | power consumption |
| | | Large contrast ratio |
| 5 | Large specific resistance | Large voltage holding ratio |
| | | and large contrast ratio |
| 6 | High stability to | Long service life |
| | ultraviolet light and heat | |
| 7 | Large elastic constant | Large contrast |
| | | ratio and short |
| | | response time |

Optical anisotropy of the composition relates to a contrast ratio in the device. According to a mode of the device, large optical anisotropy or small optical anisotropy, more specifically, suitable optical anisotropy is required. A product (Δn×d) of the optical anisotropy (Δn) of the composition and a cell gap (d) in the device is designed so as to maximize the contrast ratio. A suitable value of the product depends on a type of the operating mode. A composition having large optical anisotropy is preferred for a device having a small cell gap. Large dielectric anisotropy in the composition contributes to low threshold voltage, small electric power consumption and a large contrast ratio in the device. Accordingly, the large positive or negative dielectric anisotropy is preferred. Large specific resistance in the composition contributes to a large voltage holding ratio and the large contrast ratio in the device. Accordingly, a composition having large specific resistance in an initial stage is preferred. The composition having large specific resistance even after the device has been used for a long period of time is preferred. Stability of the composition to ultraviolet light and heat relates to a service life of the device. In the case where the stability is high, the device has a long service life. Such characteristics are preferred for an AM device use in a computer monitor, a liquid crystal television and so forth.

In a liquid crystal display device having a polymer sustained alignment (PSA) mode, a liquid crystal composition containing a polymer is used. First, a composition to which a small amount of a polymerizable compound is added is injected into the device. Next, the composition is irradiated with ultraviolet light while voltage is applied between substrates of the device. The polymerizable compound is polymerized to form a network structure of the polymer in the composition. In the composition, alignment of liquid crystal molecules can be controlled by the polymer, and therefore the response time in the device is shortened and also image persistence is improved. Such an effect of the polymer can be expected for a device having the mode such as the TN mode, the ECB mode, the OCB mode, the IPS mode, the VA mode, the FFS mode and the FPA mode.

The liquid crystal composition is prepared by mixing a liquid crystal compound. An additive such as a polymerizable compound, a polymerization initiator, a polymerization inhibitor, an optically active compound, an antioxidant, an ultraviolet light absorber, a light stabilizer, a heat stabilizer and an antifoaming agent is added to the composition when necessary. Among the additives, the light stabilizer is effective in preventing the liquid crystal compound from being decomposed by backlight, or light from the sun. A high voltage holding ratio of the device is maintained by the effect, and therefore the service life of the device is increased. Although a hindered amine light stabilizer (HALS) is suitable for such a purpose, a superb light stabilizer is expected to be developed.

### CITATION LIST

### Patent Literature

Patent literature No. 1: JP 2004-507607 A.

### Non-Patent Literature

Non-Patent literature No. 1: K. Schoening, W. Fischer, S. Hauck, A. Dichtl, and M. Kuepfert J. Org. Chem. 2009, 74, 1567-1573.

### SUMMARY OF INVENTION

The invention provides a compound having an effect on preventing photolysis of a liquid crystal composition, and high solubility in the liquid crystal composition. The invention further provides a liquid crystal composition containing the compound, and satisfying at least one of characteristics such as high maximum temperature of a nematic phase, low minimum temperature of the nematic phase, small viscosity, suitable optical anisotropy, large positive or negative dielectric anisotropy, large specific resistance, high stability to ultraviolet light, high stability to heat and a large elastic constant. The invention provides a liquid crystal composition having stability to light. The invention further provides a liquid crystal display device including the composition and having a wide temperature range in which the device can be used, a short response time, a high voltage holding ratio, low threshold voltage, a large contrast ratio, a small flicker rate and a long service life.

The invention concerns a compound represented by formula (1), a liquid crystal composition containing the compound, and a liquid crystal display device including the composition. Definitions of symbols such as R¹ are described in item 1.

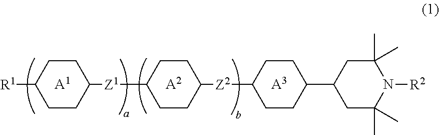

### DESCRIPTION OF EMBODIMENTS

The invention provides a compound having an effect on preventing photolysis of a liquid crystal composition, and high solubility in the liquid crystal composition. The invention further provides a liquid crystal composition containing the compound, and satisfying at least one of characteristics such as high maximum temperature of a nematic phase, low minimum temperature of the nematic phase, small viscosity, suitable optical anisotropy, large positive or negative dielectric anisotropy, large specific resistance, high stability to ultraviolet light, high stability to heat and a large elastic constant. The invention provides a liquid crystal composition having stability to light. The invention further provides a liquid crystal display device including the composition and having a wide temperature range in which the device can be used, a short response time, a high voltage holding ratio, low threshold voltage, a large contrast ratio, a small flicker rate and a long service life.

Usage of terms herein is as described below. Terms “liquid crystal compound,” “liquid crystal composition” and “liquid crystal display device” may be occasionally abbreviated as “compound,” “composition” and “device,” respectively. “Liquid crystal compound” is a generic term for a compound having a liquid crystal phase such as a nematic phase and a smectic phase, and a compound having no liquid crystal phase but to be added for the purpose of adjusting physical properties of a composition such as maximum temperature, minimum temperature, viscosity and dielectric anisotropy. The compound has a six-membered ring such as 1,4-cyclohexylene and 1,4-phenylene, and has rod-like molecular structure. “Liquid crystal display device” is a generic term for a liquid crystal display panel and a liquid crystal display module. “Polymerizable compound” is a compound to be added for the purpose of forming a polymer in the composition.

The liquid crystal composition is prepared by mixing a plurality of liquid crystal compounds. An additive is added to the composition for the purpose of further adjusting the physical properties. The additive such as the polymerizable compound, a polymerization initiator, a polymerization inhibitor, an optically active compound, an antioxidant, an ultraviolet light absorber, a light stabilizer, a heat stabilizer, a dye and an antifoaming agent is added thereto when necessary. The liquid crystal compound and the additive are mixed in such a procedure. A proportion of the liquid crystal compound is expressed in terms of weight percent (% by weight) based on the weight of the liquid crystal composition containing no additive even after the additive has been added. A proportion of the additive is expressed in terms of weight percent (% by weight) based on the weight of the liquid crystal composition containing no additive. More specifically, a proportion of the liquid crystal compound or the additive is calculated based on the total weight of the liquid crystal compound. Weight parts per million (ppm) may be occasionally used. A proportion of the polymerization initiator and the polymerization inhibitor is exceptionally expressed based on the weight of the polymerizable compound.

“Clearing point” is a transition temperature between the liquid crystal phase and an isotropic phase in the liquid crystal compound. “Minimum temperature of the liquid crystal phase” is a transition temperature between a solid and the liquid crystal phase (the smectic phase, the nematic phase or the like) in the liquid crystal compound. “Maximum temperature of the nematic phase” is a transition temperature between the nematic phase and the isotropic phase in a mixture of the liquid crystal compound and a base liquid crystal or in the liquid crystal composition, and may be occasionally abbreviated as “maximum temperature.” “Minimum temperature of the nematic phase” may be occasionally abbreviated as “minimum temperature.” An expression “increase the dielectric anisotropy” means that a value of dielectric anisotropy positively increases in a composition having positive dielectric anisotropy, and the value of dielectric anisotropy negatively increases in a composition having negative dielectric anisotropy. An expression “having a large voltage holding ratio” means that the device has a large voltage holding ratio at room temperature and also at a temperature close to the maximum temperature in an initial stage, and the device has the large voltage holding ratio at room temperature and also at a temperature close to the maximum temperature even after the device has been used for a long period of time. In order to examine characteristics of the composition and the device, an aging test may be occasionally carried out.

A compound represented by formula (1) may be occasionally abbreviated as compound (1). At least one compound selected from the group of compounds represented by formula (1) may be occasionally abbreviated as compound (1). “Compound (1)” means one compound, a mixture of two compounds or a mixture of three or more compounds represented by formula (1). A same rule applies also to any other compound represented by any other formula. In formulas (1) to (15), a symbol of A¹, B¹, C¹ or the like surrounded by a hexagonal shape corresponds to ring A¹, ring B¹ and ring C¹, respectively. The hexagonal shape represents a six-membered ring such as cyclohexane or benzene. The hexagonal shape may occasionally represent a fused ring such as naphthalene or a bridged ring such as adamantane.

A symbol of terminal group R¹¹ is used in a plurality of compounds in chemical formulas of component compounds. In the compounds, two groups represented by two pieces of arbitrary R¹¹ may be identical or different. For example, in one case, R¹¹ of compound (2) is ethyl and R¹¹ of compound (3) is ethyl. In another case, R¹¹ of compound (2) is ethyl and R¹¹ of compound (3) is propyl. A same rule applies also to a symbol of R¹², R₁₃, Z¹¹ or the like. In compound (8), when a subscript i is 2, two of ring D¹ exists. In the compound, two groups represented by two of ring D¹ may be identical or different. A same rule applies also to two of arbitrary ring D¹ when the subscript i is larger than 2. A same rule applies also to other symbols. In formula (1a) or the like, a straight line crossing one side of a benzene ring represents that arbitrary hydrogen on the ring may be replaced by fluorine. A subscript such as ‘c’ represents the number of groups to be replaced. When subscript ‘c’ is 0, no such replacement exists.

An expression “at least one piece of ‘A’” means that the number of ‘A’ is arbitrary. An expression “at least one piece of ‘A’ may be replaced by ‘B’” means that, when the number of ‘A’ is 1, a position of ‘A’ is arbitrary, and also when the number of ‘A’ is 2 or more, positions thereof can be selected without restriction. A same rule applies also to an expression “at least one piece of ‘A’ is replaced by ‘B’.” An expression “at least one piece of ‘A’ may be replaced by ‘B’, ‘C’ or ‘D’” includes a case where arbitrary ‘A’ is replaced by ‘B’, a case where arbitrary ‘A’ is replaced by ‘C’, and a case where arbitrary ‘A’ is replaced by ‘D’, and also a case where a plurality of pieces of ‘A’ are replaced by at least two pieces of ‘B’, ‘C’ and/or ‘D.’ For example, “alkyl in which at least one piece of —CH₂— may be replaced by —O— or —CH═CH—” includes alkyl, alkoxy, alkoxyalkyl, alkenyl, alkoxyalkenyl and alkenyloxyalkyl. In addition, a case where two pieces of consecutive —CH₂— are replaced by —O— to form —O—O— is not preferred. In alkyl or the like, a case where —CH₂— of a methyl part (—CH₂—H) is replaced by —O— to form —O—H is not preferred, either.

An expression “R¹¹ and R¹² are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine” may be occasionally used. In the expression, “in the groups” may be interpreted according to wording. In the expression, “the groups” means alkyl, alkenyl, alkoxy, alkenyloxy or the like. More specifically, “the groups” represents all of the groups described before the term “in the groups.”

Alkyl of the liquid crystal compound is straight-chain alkyl or branched-chain alkyl, but includes no cyclic alkyl. In general, straight-chain alkyl is preferred to branched-chain alkyl. A same rule applies also to a terminal group such as alkoxy and alkenyl. With regard to a configuration of 1,4-cyclohexylene, trans is preferred to cis for increasing the maximum temperature. Then, 2-fluoro-1,4-phenylene means two divalent groups described below. In a chemical formula, fluorine may be leftward (L) or rightward (R). A same rule applies also to an asymmetrical divalent group formed by removing two hydrogens from a ring, such as tetrahydropyran-2,5-diyl.

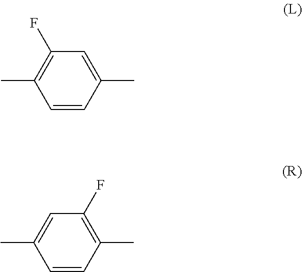

The invention includes items described below.

Item 1. A compound, represented by formula (1):

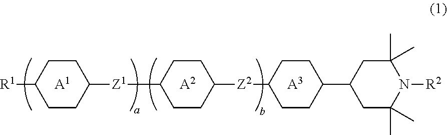

wherein, in formula (1),

R¹ is alkyl having 1 to 10 carbons, alkoxy having 1 to 10 carbons or alkenyl having 2 to 10 carbons, and in the groups, at least one hydrogen may be replaced by fluorine or chlorine;

R² is hydrogen, hydroxy, oxy radical, alkyl having 1 to 10 carbons, alkoxy having 1 to 10 carbons or alkenyl having 2 to 10 carbons, and in the groups, at least one hydrogen bound to carbon may be replaced by fluorine or chlorine;

ring A¹, ring A² and ring A³ are independently 1,4-cyclohexylene, 1,4-cyclohexenylene, 3,4-dihydro-2H-pyrane-2,5-diyl, 3,4-dihydro-2H-pyrane-3,6-diyl, 3,6-dihydro-2H-pyrane-2,5-diyl, tetrahydropyran-2,5-diyl, 1,3-dioxane-2,5-diyl, 1,4-phenylene, pyridine-2,5-diyl, decahydronaphthalene-2,6-diyl, naphthalene-1,3-diyl, naphthalene-1,4-diyl, naphthalene-1,5-diyl, naphthalene-1,6-diyl, naphthalene-1,7-diyl, naphthalene-1,8-diyl, naphthalene-2,3-diyl, naphthalene-2,6-diyl or naphthalene-2,7-diyl, and in the rings, at least one hydrogen on an aromatic ring may be replaced by fluorine, chlorine, cyano, alkyl having 1 to 5 carbons, alkoxy having 1 to 5 carbons, alkyl having 1 to 5 carbons in which at least one hydrogen is replaced by fluorine or chlorine, or alkoxy having 1 to 5 carbons in which at least one hydrogen is replaced by fluorine or chlorine;

Z¹ and Z² are independently a single bond or alkylene having 1 to 10 carbons, and in the alkylene, at least one piece of —CH₂— may be replaced by —O—, —COO— or —OCO—, and at least one piece of —CH₂—CH₂— may be replaced by —CH═CH— or —C≡C—, and in the groups, at least one hydrogen may be replaced by fluorine or chlorine; and

a and b are independently 0 or 1.

Item 2. The compound according to item 1, wherein, in formula (1), R¹ is alkyl having 1 to 10 carbons, alkoxy having 1 to 10 carbons or alkenyl having 2 to 10 carbons; R² is hydrogen, hydroxy, oxy radical, alkyl having 1 to 10 carbons, alkoxy having 1 to 10 carbons or alkenyl having 2 to 10 carbons; ring A¹, ring A² and ring A³ are independently 1,4-cyclohexylene, 1,4-cyclohexenylene, tetrahydropyran-2,5-diyl, 1,3-dioxane-2,5-diyl, 1,4-phenylene, pyridine-2,5-diyl or naphthalene-2,6-diyl, and in rings, at least one hydrogen on an aromatic ring may be replaced by fluorine, chlorine, —CF₃, —CHF₂, —CH₂F, —OCF₃, —OCHF₂ or —OCH₂F; Z¹ and Z² are independently a single bond, —COO—, —OCO—, —CH₂O—, —OCH₂—, —CF₂O—, —OCF₂—, —CH₂CH₂—, —CF₂CF₂—, —CH═CH—, —CF═CF—, —CH₂CH₂CH₂CH₂— or —CH₂CH═CHCH₂—; and a and b are independently 0 or 1.

Item 3. The compound according to item 1, wherein, in formula (1), R¹ is alkyl having 1 to 10 carbons, alkoxy having 1 to 10 carbons or alkenyl having 2 to 10 carbons; R² is hydrogen, hydroxy, oxy radical, alkyl having 1 to 10 carbons, alkoxy having 1 to 10 carbons or alkenyl having 2 to 10 carbons; ring A¹, ring A² and ring A³ are independently 1,4-cyclohexylene, 1,4-cyclohexenylene, tetrahydropyran-2,5-diyl, 1,3-dioxane-2,5-diyl, 1,4-phenylene or pyridine-2,5-diyl, and in the rings, at least one hydrogen on an aromatic ring may be replaced by fluorine or chlorine; Z¹ and Z² are independently a single bond, —COO—, —CH₂O—, —CF₂O—, —CH₂CH₂— or —CH═CH—; and a and b are independently 0 or 1.

Item 4. The compound according to item 1, wherein, in formula (1), R¹ is alkyl having 1 to 8 carbons, alkenyl having 2 to 8 carbons or alkoxy having 2 to 8 carbons; R² is hydrogen, hydroxy, oxy radical, alkyl having 1 to 8 carbons, alkoxy having 1 to 8 carbons or alkenyl having 2 to 8 carbons; ring A¹, ring A² and ring A³ are independently 1,4-cyclohexylene, 1,4-phenylene, or 1,4-phenylene in which at least one hydrogen is replaced by fluorine; Z¹ and Z² are independently a single bond, —COO—, —CH₂O— or —CH₂CH₂—; and a and b are independently 0 or 1.

Item 5. The compound according to item 1, represented by formula (1a) or (1b):

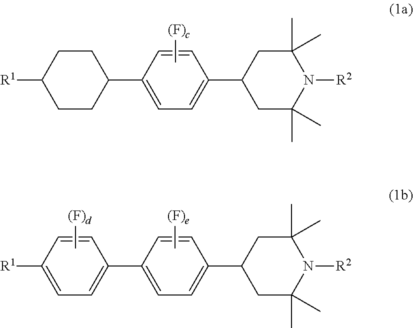

wherein, in formula (1a) or (1b), R¹ is straight-chain alkyl having 1 to 8 carbons, straight-chain alkoxy having 2 to 8 carbons or straight-chain alkenyl having 2 to 8 carbons; R² is hydrogen, hydroxy, oxy radical, straight-chain alkyl having 1 to 8 carbons, straight-chain alkoxy having 1 to 8 carbons or straight-chain alkenyl having 2 to 8 carbons; and c, d and e are independently an integer from 0 to 4.

Item 6. The compound according to item 5, wherein, in formula (1a) or (1b), R¹ is straight-chain alkyl having 1 to 8 carbons; R² is hydrogen, hydroxy, oxy radical or straight-chain alkyl having 1 to 8 carbons; and c, d and e are independently 0 or 1.

Item 7. The compound according to item 1, represented by formula (1c) or (1d):

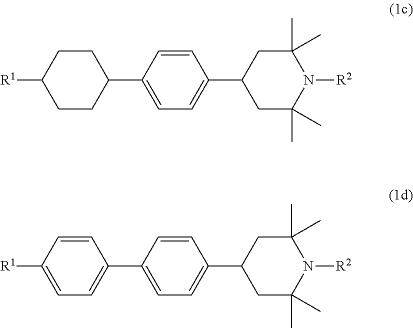

wherein, in formula (1c) or (1d), R¹ is straight-chain alkyl having 1 to 6 carbons, straight-chain alkoxy having 1 to 6 carbons or straight-chain alkenyl having 2 to 6 carbons; and R² is hydrogen, hydroxy, oxy radical, straight-chain alkyl having 1 to 6 carbons, straight-chain alkoxy having 1 to 6 carbons or straight-chain alkenyl having 2 to 6 carbons.

Item 8. The compound according to item 7, wherein, in formula (1c) or (1d), R¹ is straight-chain alkyl having 1 to 6 carbons; and R² is hydrogen, hydroxy, oxy radical or straight-chain alkyl having 1 to 6 carbons.

Item 9. A liquid crystal composition, containing at least one compound according to any one of items 1 to 8.

Item 10. The liquid crystal composition according to item 9, further containing at least one compound selected from the group of compounds represented by formulas (2) to (4):

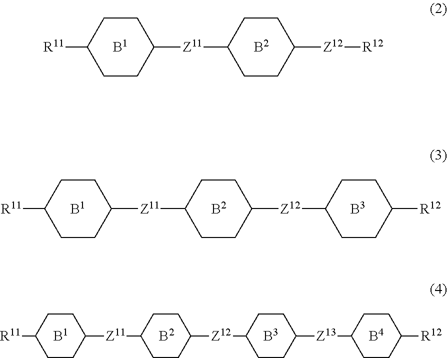

wherein, in formulas (2) to (4),

R¹¹ and R¹² are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine;

ring B¹, ring B², ring B³ and ring B⁴ are independently 1,4-cyclohexylene, 1,4-phenylene, 2-fluoro-1,4-phenylene, 2,5-difluoro-1,4-phenylene or pyrimidine-2,5-diyl; and

Z¹¹, Z¹² and Z¹³ are independently a single bond, —COO—, —CH₂CH₂—, —CH═CH— or —C≡C—.

Item 11. The liquid crystal composition according to item 9 or 10, further containing at least one compound selected from the group of compounds represented by formulas (5) to (7):

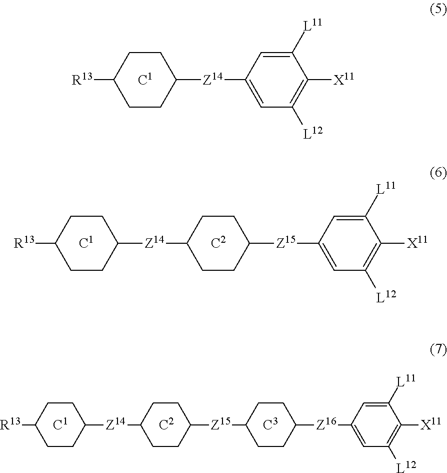

wherein, in formulas (5) to (7),

R¹³ is alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine;

X¹¹ is fluorine, chlorine, —OCF₃, —OCHF₂, —CF₃, —CHF₂, —CH₂F, —OCF₂CHF₂ or —OCF₂CHFCF₃;

ring C¹, ring C² and ring C³ are independently 1,4-cyclohexylene, 1,4-phenylene, 1,4-phenylene in which at least one hydrogen is replaced by fluorine, tetrahydropyran-2,5-diyl, 1,3-dioxane-2,5-diyl, or pyrimidine-2,5-diyl;

Z¹⁴, Z¹⁵ and Z¹⁶ are independently a single bond, —COO—, —OCO—, —CH₂O—, —OCH₂—, —CF₂O—, —OCF₂—, —CH₂CH₂—, —CH═CH—, —C≡C— or —(CH₂)₄—; and

L¹¹ and L¹² are independently hydrogen or fluorine.

Item 12. The liquid crystal composition according to any one of items 9 to 11, further containing at least one compound selected from the group of compounds represented by formula (8):

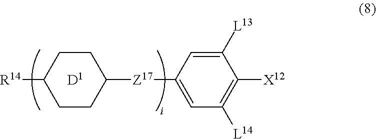

wherein, in formula (8),

R¹⁴ is alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine;

X¹² is —C≡N or —C≡C—C≡N;

ring D¹ is independently 1,4-cyclohexylene, 1,4-phenylene, 1,4-phenylene in which at least one hydrogen is replaced by fluorine, tetrahydropyran-2,5-diyl, 1,3-dioxane-2,5-diyl, or pyrimidine-2,5-diyl;

Z¹⁷ is independently a single bond, —COO—, —OCO—, —CH₂O—, —OCH₂—, —CF₂O—, —OCF₂—, —CH₂CH₂— or —C≡C—;

L¹³ and L¹⁴ are independently hydrogen or fluorine; and

i is 1, 2, 3 or 4.

Item 13. The liquid crystal composition according to any one of items 9 to 12, further containing at least one compound selected from the group of compounds represented by formulas (9) to (15):

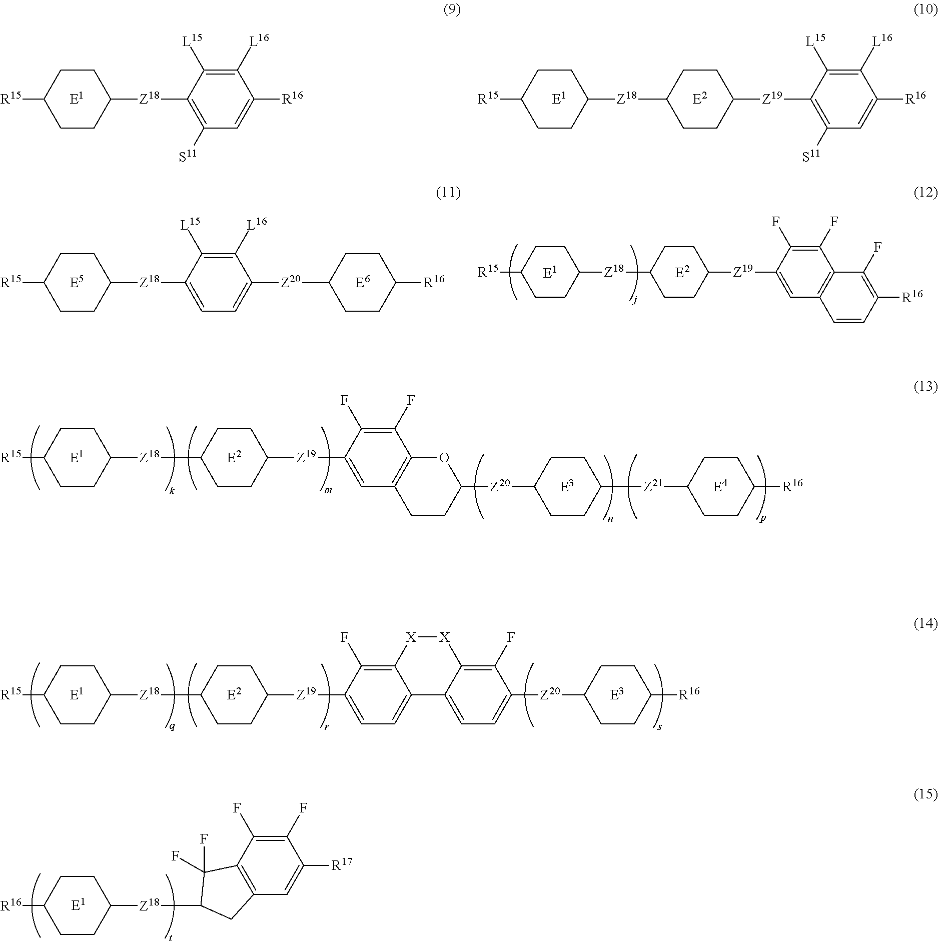

wherein, in formulas (9) to (15),

R¹⁵, R¹⁶ and R¹⁷ are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine, and R¹⁷ may be hydrogen or fluorine;

ring E¹, ring E², ring E³ and ring E⁴ are independently 1,4-cyclohexylene, 1,4-cyclohexenylene, 1,4-phenylene, 1,4-phenylene in which at least one hydrogen is replaced by fluorine, tetrahydropyran-2,5-diyl or decahydronaphthalene-2,6-diyl;

ring E⁵ and ring E⁶ are independently 1,4-cyclohexylene, 1,4-cyclohexenylene, 1,4-phenylene, tetrahydropyran-2,5-diyl or decahydronaphthalene-2,6-diyl;

Z¹⁸, Z¹⁹, Z²⁰ and Z²¹ are independently a single bond, —COO—, —OCO—, —CH₂O—, —OCH₂—, —CF₂O—, —OCF₂—, —CH₂CH₂—, —CF₂OCH₂CH₂— or —OCF₂CH₂CH₂—;

L¹⁵ and L¹⁶ are independently fluorine or chlorine;

S¹¹ is hydrogen or methyl;

X is —CHF— or —CF₂—; and

j, k, m, n, p, q, r and s are independently 0 or 1, a sum of k, m, n and p is 1 or 2, a sum of q, r and s is 0, 1, 2 or 3, and t is 1, 2 or 3.

Item 14. A liquid crystal display device, including at least one liquid crystal composition according to any one of items 9 to 13.

The invention further includes the following items: (a) the liquid crystal composition, further containing one, two or at least three additives such as a polymerizable compound, a polymerization initiator, a polymerization inhibitor, an optically active compound, an antioxidant, an ultraviolet light absorber, a light stabilizer different from compound (1), a heat stabilizer and an antifoaming agent; (b) a polymerizable composition prepared by adding the polymerizable compound to the liquid crystal composition; (c) a liquid crystal composite prepared by polymerizing the polymerizable composition; (d) a liquid crystal display device having a polymer sustained alignment (PSA) mode including the liquid crystal composite; (e) Use as the light stabilizer of compound (1); (f) use as the heat stabilizer of compound (1); (g) use as a combination of the light stabilizer different from compound (1) and compound (1); and (h) use as an optically active composition by adding the optically active compound to the liquid crystal composition.

An aspect of compound (1), synthesis of compound (1), the liquid crystal composition and the liquid crystal display device will be described in the order.

### 1. Aspect of Compound (1)

Compound (1) of the invention has features of having tetramethyl piperidinyl at a terminal and having a rod-like skeleton. The compound is useful as a hindered amine light stabilizer. Compound (1) is suitable for trapping a decomposition product formed by photoreaction of the liquid crystal compound. The compound can be added to a mixture of the liquid crystal compounds, namely, the liquid crystal composition. The reason is that the compound has high solubility in the liquid crystal composition. Compound (1) is effective in preventing the liquid crystal compound from being decomposed by backlight or light from the sun. Compound (1) has presumably has an effect as the heat stabilizer.

If the liquid crystal display device is used for a long period of time, the liquid crystal compound tends to be decomposed by light to form the decomposition product. The product is an impurity, and therefore is not preferred for the device. The reason is that the impurity causes a phenomenon such as reduction of a contrast ratio, generation of display unevenness and image persistence. The phenomenon can be easily visually identified, and is also very marked even if a degree thereof is slight. Accordingly, a light stabilizer from which an amount of generation of the impurity is smaller even by 1% in comparison with a conventional light stabilizer is preferred. Compound (1) is such a light stabilizer.

Preferred examples of compound (1) will be described. Preferred examples of substituent R, ring A and bonding group Z in compound (1) apply also to a subordinate formula of formula (1) for compound (1). In compound (1), characteristics can be arbitrarily adjusted by suitably combining kinds of the groups. Compound (1) may contain a larger amount of isotope such as ²H (deuterium) and ¹³C than an amount of natural abundance because no significant difference exists in the characteristics of the compound.

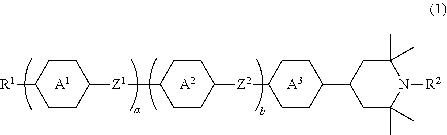

In formula (1), R¹ is alkyl having 1 to 10 carbons, alkoxy having 1 to 10 carbons or alkenyl having 2 to 10 carbons, and in the groups, at least one hydrogen may be replaced by fluorine or chlorine.

Preferred alkyl is methyl, ethyl, propyl, butyl, pentyl, hexyl, heptyl or octyl. Further preferred alkyl is methyl, ethyl, propyl, butyl or pentyl for decreasing the viscosity.

Preferred alkoxy is methoxy, ethoxy, propoxy, butoxy, pentyloxy, hexyloxy or heptyloxy. Further preferred alkoxy is methoxy or ethoxy for decreasing the viscosity.

Preferred alkenyl is vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl or 5-hexenyl. Further preferred alkenyl is vinyl, 1-propenyl, 3-butenyl or 3-pentenyl for decreasing the viscosity. A preferred configuration of —CH═CH— in the alkenyl depends on a position of a double bond. Trans is preferred in alkenyl such as 1-propenyl, 1-butenyl, 1-pentenyl, 1-hexenyl, 3-pentenyl and 3-hexenyl for decreasing the viscosity, for instance. Cis is preferred in alkenyl such as 2-butenyl, 2-pentenyl and 2-hexenyl.

Preferred examples of alkyl in which at least one hydrogen is replaced by fluorine or chlorine include fluoromethyl, 2-fluoroethyl, 3-fluoropropyl, 4-fluorobutyl, 5-fluoropentyl, 6-fluorohexyl, 7-fluoroheptyl or 8-fluorooctyl. Further preferred examples include fluoromethyl, 2-fluoroethyl, 3-fluoropropyl, 4-fluorobutyl or 5-fluoropentyl for increasing the dielectric anisotropy.

Preferred examples of alkenyl in which at least one hydrogen is replaced by fluorine or chlorine include 2,2-difluorovinyl, 3,3-difluoro-2-propenyl, 4,4-difluoro-3-butenyl, 5,5-difluoro-4-pentenyl or 6,6-difluoro-5-hexenyl. Further preferred examples include 2,2-difluorovinyl or 4,4-difluoro-3-butenyl for decreasing the viscosity.

In formula (1), R² is hydrogen, hydroxy, oxy radical, alkyl having 1 to 10 carbons, alkoxy having 1 to 10 carbons or alkenyl having 2 to 10 carbons, and in the groups, at least one hydrogen bound to carbon may be replaced by fluorine or chlorine. Hydroxy means —OH, and the oxy radical means a free radical having a part of >N—O—.. Preferred R² is hydrogen, hydroxy, oxy radical, alkyl having 1 to 3 carbons or alkoxy having 1 to 3 carbons. Further preferred R² is hydrogen, hydroxy, oxy radical, methyl, ethyl, propyl, isopropyl, methoxy or ethoxy. Particularly preferred R² is hydrogen, hydroxy, oxy radical, methyl or methoxy. Most preferred R² is hydrogen, methyl or methoxy. Most preferred R² is also hydrogen, hydroxy or methyl. Most preferred R² is also hydroxy, methyl or methoxy.

In formula (1), ring A¹, ring A² and ring A³ are independently 1,4-cyclohexylene, 1,4-cyclohexenylene, 3,4-dihydro-2H-pyrane-2,5-diyl, 3,4-dihydro-2H-pyrane-3,6-diyl, 3,6-dihydro-2H-pyrane-2,5-diyl, tetrahydropyran-2,5-diyl, 1,3-dioxane-2,5-diyl, 1,4-phenylene, pyridine-2,5-diyl, decahydronaphthalene-2,6-diyl, naphthalene-1,3-diyl, naphthalene-1,4-diyl, naphthalene-1,5-diyl, naphthalene-1,6-diyl, naphthalene-1,7-diyl, naphthalene-1,8-diyl, naphthalene-2,3-diyl, naphthalene-2,6-diyl or naphthalene-2,7-diyl, and in the rings, at least one hydrogen on an aromatic ring may be replaced by fluorine, chlorine, cyano, alkyl having 1 to 5 carbons, alkoxy having 1 to 5 carbons, alkyl having 1 to 5 carbons in which at least one hydrogen is replaced by fluorine or chlorine, or alkoxy having 1 to 5 carbons in which at least one hydrogen is replaced by fluorine or chlorine.

Then, 3,4-dihydro-2H-pyrane-2,5-diyl, 3,4-dihydro-2H-pyrane-3,6-diyl and 3,6-dihydro-2H-pyrane-2,5-diyl mean the following divalent group or divalent group having a mirror image thereof in the order.

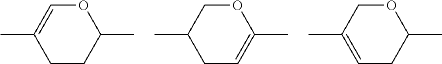

Preferred ring A¹, ring A² or ring A³ is 1,4-cyclohexylene, 1,4-cyclohexenylene, tetrahydropyran-2,5-diyl, 1,3-dioxane-2,5-diyl, 1,4-phenylene, pyridine-2,5-diyl or naphthalene-2,6-diyl. In the rings, at least one hydrogen on an aromatic ring may be replaced by fluorine, chlorine, —CF₃, —CHF₂, —CH₂F, —OCF₃, —OCHF₂ or —OCH₂F. Further preferred ring A¹, ring A² or ring A³ is 1,4-cyclohexylene, 1,4-cyclohexenylene, tetrahydropyran-2,5-diyl, 1,3-dioxane-2,5-diyl, 1,4-phenylene or pyridine-2,5-diyl, and in the rings, at least one hydrogen on an aromatic ring may be replaced by fluorine or chlorine. Particularly preferred ring A¹, ring A² or ring A³ is 1,4-cyclohexylene, 1,4-phenylene, or 1,4-phenylene in which at least one hydrogen is replaced by fluorine. Most preferred ring A¹, ring A² or ring A³ is 1,4-phenylene.

In formula (1), Z¹ and Z² are independently a single bond or alkylene having 1 to 10 carbons, and in the alkylene, at least one piece of —CH₂— may be replaced by —O—, —COO— or —OCO—, and at least one piece of —CH₂—CH₂— may be replaced by —CH═CH— or —C≡C—, and in the groups, at least one hydrogen may be replaced by fluorine or chlorine.

Preferred Z¹ or Z² is a single bond, —COO—, —OCO—, —CH₂O—, —OCH₂—, —CF₂O—, —OCF₂—, —CH₂CH₂—, —CF₂CF₂—, —CH═CH—, —CF═CF—, —CH₂CH₂CH₂CH₂— or —CH₂CH═CHCH₂—. Further preferred Z¹ or Z² is a single bond, —COO—, —CH₂O—, —CF₂O—, —CH₂CH₂— or —CH═CH—. Particularly preferred Z¹ or Z² is a single bond, —COO—, —CH₂O— or —CH₂CH₂—. Most Preferred Z¹ or Z² is a single bond.

In formula (1), subscripts a and b are independently 0 or 1. More specifically, the number of rings is 2 to 4. The preferred number of rings is 2 or 3. The further preferred number of rings is 3.

Compound (1) having objective characteristics can be obtained by suitably selecting a combination of substituent R, ring A, bonding group Z and subscripts a and b with reference to the preferred examples described above. Preferred examples of compound (1) include the compound described in item 2, item 3 or the like.

### 2. Synthesis of Compound (1)

A synthesis method of compound (1) will be described. Compound (1) can be prepared by suitably combining methods in synthetic organic chemistry. The method is described in books such as Houben-Wyle, Methoden der Organische Chemie (Georg-Thieme Verlag, Stuttgart), Organic Syntheses (John Wily & Sons, Inc.), Organic Reactions (John Wily & Sons Inc.), Comprehensive Organic Synthesis (Pergamon Press), and New Experimental Chemistry Course (Shin Jikken Kagaku Koza in Japanese) (Maruzen Co., Ltd.).

### 2-1. Formation of Bonding Group Z

First, a scheme is shown with regard to a method for forming bonding group Z¹ or Z². Next, reactions described in the scheme will be described in methods (1) to (11). In the scheme, MSG¹ (or MSG²) is a monovalent organic group having at least one ring. The monovalent organic groups represented by a plurality of MSG¹ (or MSG²) used in the scheme may be identical or different. Compounds (1A) to (1J) correspond to compound (1).

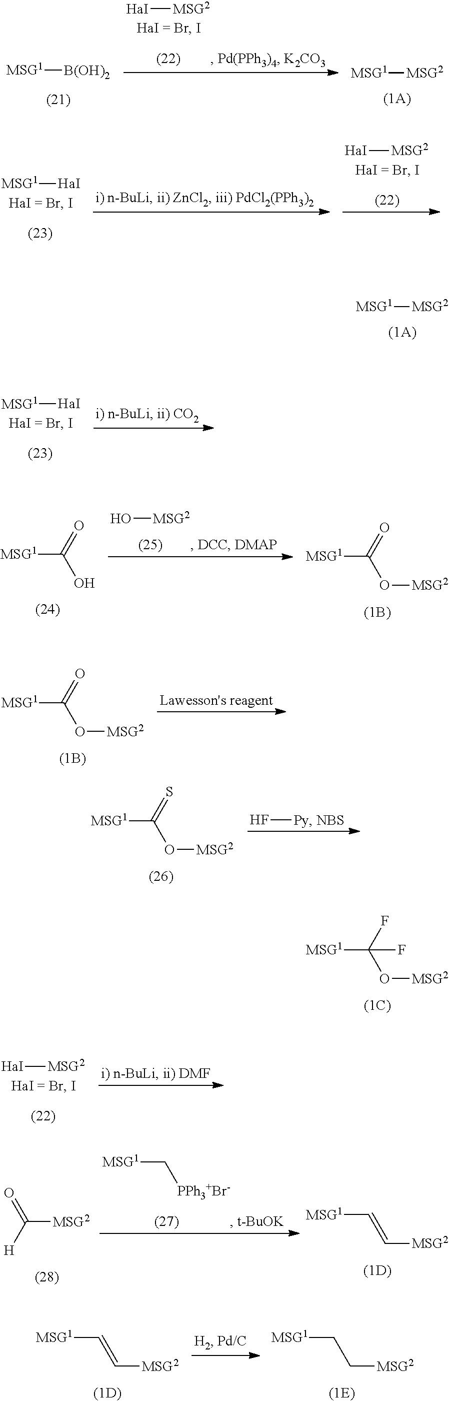

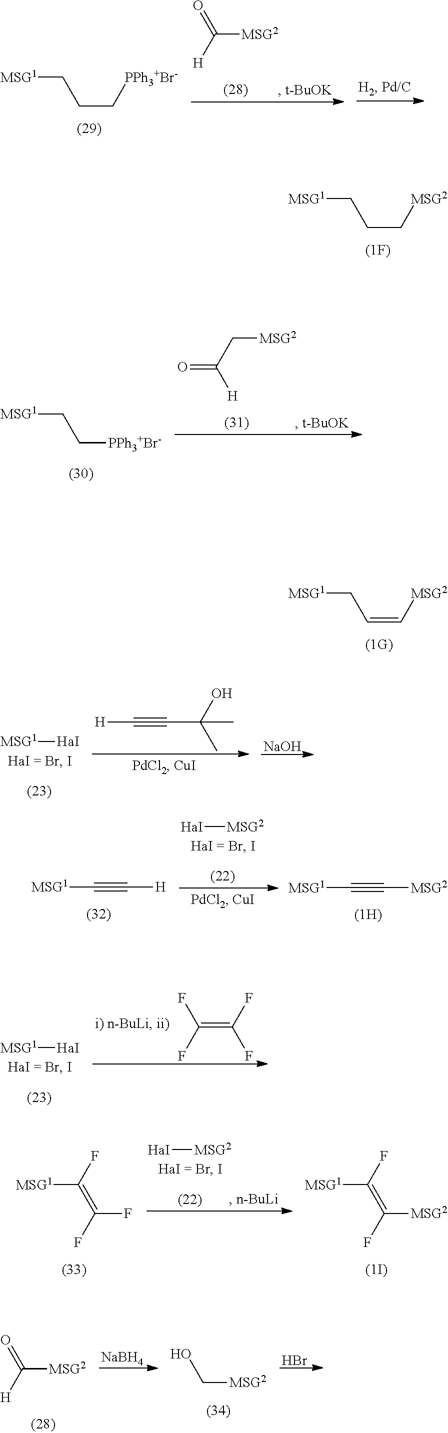

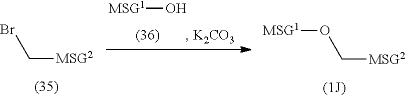

### (1) Formation of a Single Bond

Compound (1A) is prepared by allowing aryl boronic acid (21) prepared according to a publicly known method to react with halide (22), in the presence of carbonate and a catalyst such as tetrakis(triphenylphosphine) palladium. Compound (1A) is also prepared by allowing halide (23) prepared according to a publicly known method to react with n-butyllithium and subsequently with zinc chloride, and further with halide (22) in the presence of a catalyst such as dichlorobis(triphenylphosphine)palladium.

### (2) Formation of —COO—

Carboxylic acid (24) is obtained by allowing halide (23) to react with n-butyllithium and subsequently with carbon dioxide. Compound (1B) is prepared by dehydration of compound (25) prepared according to a publicly known method and carboxylic acid (24) in the presence of 1,3-dicyclohexylcarbodiimide (DCC) and 4-dimethylaminopyridine (DMAP).

### (3) Formation of —CF₂O—

Thionoester (26) is obtained by treating compound (1B) with a thiation reagent such as Lawesson's reagent. Compound (1C) is prepared by fluorinating thionoester (26) with a hydrogen fluoride-pyridine complex and N-bromosuccinimide (NBS). Refer to M. Kuroboshi et al., Chem. Lett., 1992, 827. Compound (1C) is also prepared by fluorinating thionoester (26) with (diethylamino) sulfur trifluoride (DAST). Refer to W. H. Bunnelle et al., J. Org. Chem. 1990, 55, 768. The bonding group can also be formed according to a method described in Peer. Kirsch et al., Angew. Chem. Int. Ed. 2001, 40, 1480.

### (4) Formation of —CH═CH—

Aldehyde (28) is obtained by treating halide (22) with n-butyllithium and then allowing the treated halide to react with formamide such as N, N-dimethylformamide (DMF). Phosphorus ylide is generated by treating phosphonium salt (27) prepared according to a publicly known method with a base such as potassium t-butoxide. Compound (1D) is prepared by allowing the phosphorus ylide to react with aldehyde (28). A cis isomer may be generated depending on reaction conditions, and therefore the cis isomer is isomerized into a trans isomer according to a publicly known method when necessary.

(5) Formation of —CH₂CH₂—

Compound (1E) is prepared by hydrogenating compound (1D) in the presence of a catalyst such as palladium on carbon.

(6) Formation of —(CH₂)₄—

A compound having —(CH₂)₂—CH═CH— is obtained by using phosphonium salt (29) in place of phosphonium salt (27) according to the method in method (4). Compound (1F) is prepared by performing catalytic hydrogenation of the compound obtained.

(7) Formation of —CH₂CH═CHCH₂—

Compound (1G) is prepared according to method (4) by using phosphonium salt (30) in place of phosphonium salt (27), and aldehyde (31) in place of aldehyde (28). A trans isomer may be generated depending on reaction conditions, and therefore the trans isomer is isomerized into a cis isomer according to a publicly known method when necessary.

### (8) Formation of —C≡C—

Compound (32) is obtained by allowing halide (23) to react with 2-methyl-3-butyn-2-ol in the presence of a catalyst of dichloropalladium and copper halide, and then performing deprotection under basic conditions. Compound (1H) is prepared by allowing compound (32) to react with halide (22) in the presence of the catalyst of dichloropalladium and copper halide.

### (9) Formation of —CF═CF—

Compound (33) is obtained by treating halide (23) with n-butyllithium, and then allowing the treated halide to react with tetrafluoroethylene. Compound (1I) is prepared by treating halide (22) with n-butyllithium, and then allowing the treated halide to react with compound (33).

### (10) Formation of —OCH₂—

Compound (34) is obtained by reducing aldehyde (28) with a reducing agent such as sodium borohydride. Bromide (35) is obtained by brominating compound (34) with hydrobromic acid or the like. Compound (1J) is prepared by allowing bromide (35) to react with compound (36) in the presence of a base such as potassium carbonate.

(11) Formation of —CF₂CF₂—

A compound having —(CF₂)₂— is obtained by fluorinating diketone (—COCO—) with sulfur tetrafluoride, in the presence of a hydrogen fluoride catalyst, according to a method described in J. Am. Chem. Soc., 2001, 123, 5414.

### 2-2. Raw Material

Raw materials of a 2,2,6,6-tetramethylpiperidine ring include 4-hydroxy-2,2,6,6-tetramethylpiperidine and 2,2,6,6-tetramethyl-4-piperidone, and available from Sigma-Aldrich Co., LLC.

### 3. Liquid Crystal Composition

### 3-1. Component Compound

A liquid crystal composition of the invention will be described. The composition contains at least one compound (1) as component A. The composition may contain two, three or more compounds (1). A preferred proportion of compound (1) is about 0.01% by weight or more for maintaining high stability to ultraviolet light, and about 5% by weight or less for allowing dissolution in the liquid crystal composition, based thereon. A further preferred proportion is in the range of about 0.05% by weight to about 2% by weight based thereon. A most preferred proportion is in the range of about 0.05% by weight to about 1% by weight based thereon.

TABLE 2
Dielectric anisotropy of component compound

| Component of | Component | Dielectric |
|---|---|---|
| composition | compound | anisotropy |
| Component A | Compound (1) | — |
| Component B | Compound (2) to | Small |
| | compound (4) | |
| Component C | Compound (5) to | Positively large |
| | compound (7) | |
| Component D | Compound (8) | Positively large |
| Component E | Compound (9) to | Negatively large |
| | compound (15) | |

The composition contains compound (1) as component A, and further preferably contains a liquid crystal compound selected from components B, C, D and E shown in Table 2. When the composition is prepared, components B, C, D and E are preferably selected by taking into account the positive or negative dielectric anisotropy and magnitude of the dielectric anisotropy. The composition may contain a liquid crystal compound different from compounds (1) to (15). The composition may not contain such a liquid crystal compound.

Component B includes a compound in which two terminal groups are alkyl or the like. Specific examples of preferred component B include compounds (2-1) to (2-11), compounds (3-1) to (3-19) and compounds (4-1) to (4-7). In the compounds, R¹¹ and R¹² are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine.

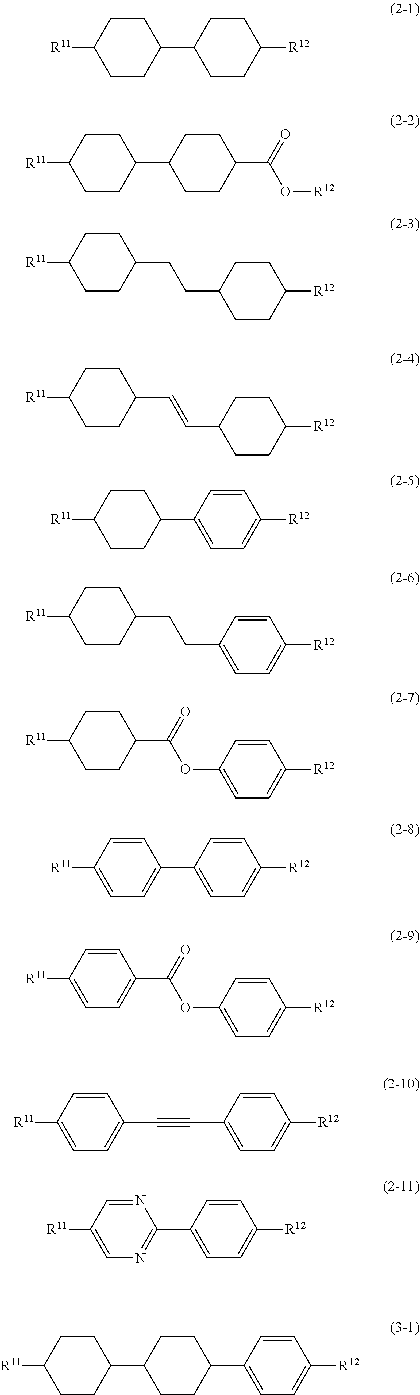

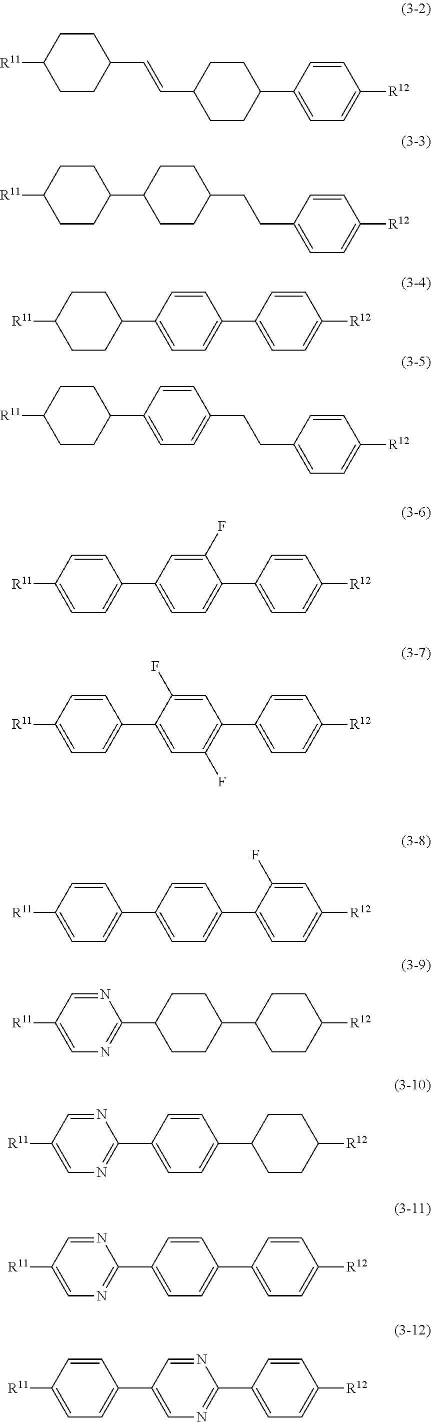

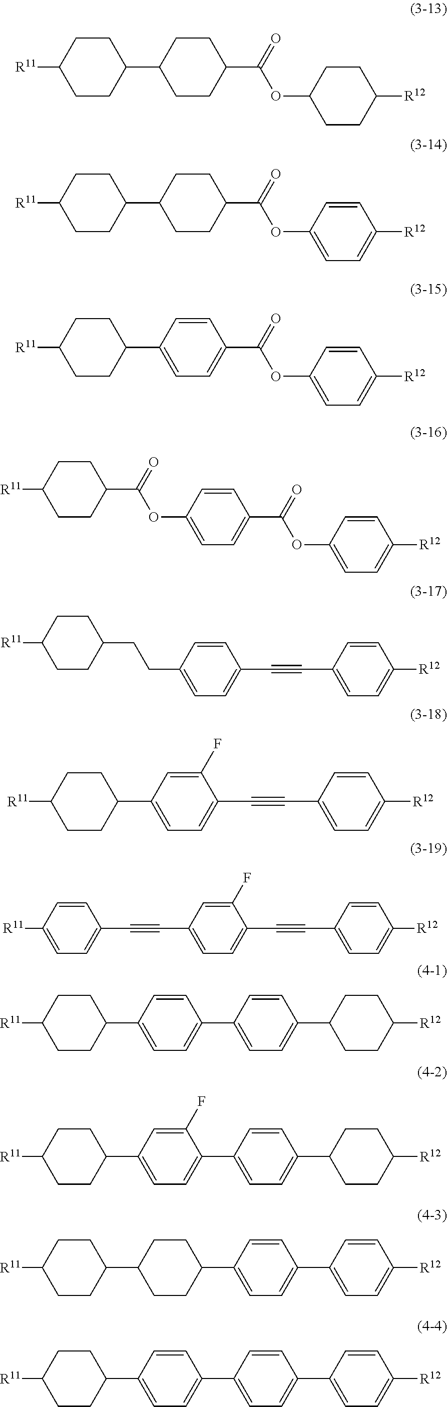

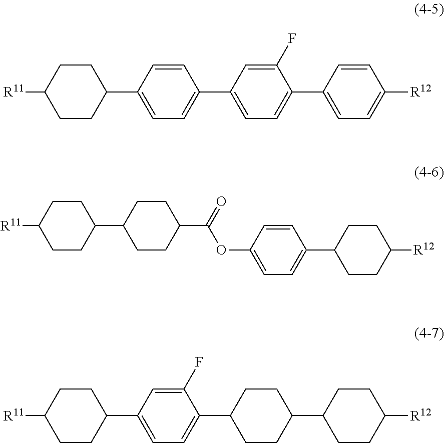

Component B has small dielectric anisotropy. Component B is close to neutrality. Compound (2) is effective in decreasing the viscosity or adjusting the optical anisotropy. Compounds (3) and (4) are effective in extending the temperature range of the nematic phase by increasing the maximum temperature, or adjusting the optical anisotropy.

As a proportion of component B is increased, the viscosity of the composition is decreased, but the dielectric anisotropy is decreased. Thus, as long as a desired value of threshold voltage of the device is met, the proportion is preferably as large as possible. When a composition for an IPS mode, a VA mode or the like is prepared, the proportion of component B is preferably about 30% by weight or more, and further preferably about 40% by weight or more, based thereon.

Component C is a compound having a halogen-containing group or a fluorine-containing group at a right terminal. Specific examples of preferred component C include compounds (5-1) to (5-16), compounds (6-1) to (6-113) and compounds (7-1) to (7-57). In the compounds, R¹³ is alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine. X¹¹ is fluorine, chlorine, —OCF₃, —OCHF₂, —CF₃, —CHF₂, —CH₂F, —OCF₂CHF₂ or —OCF₂CHFCF₃.

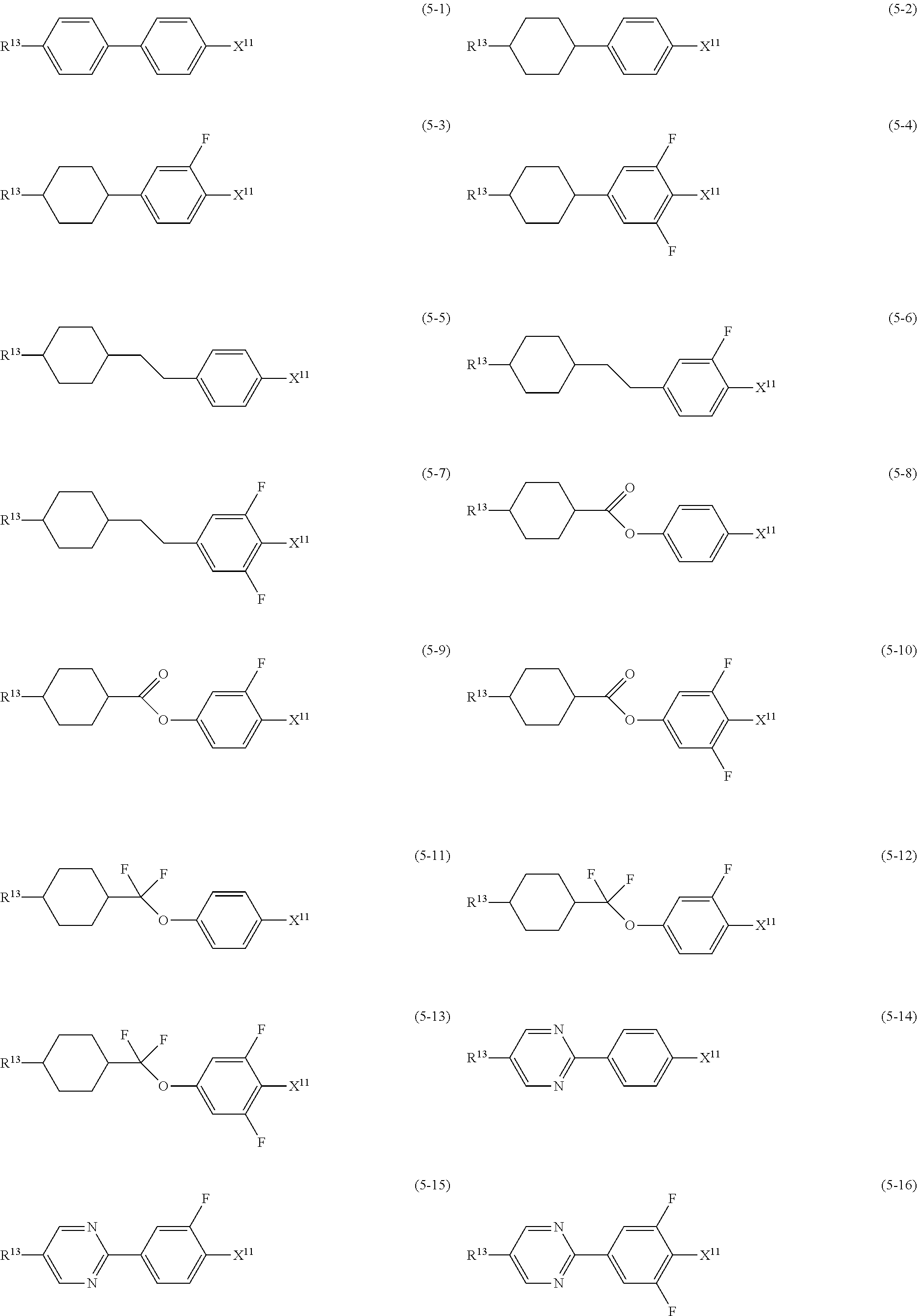

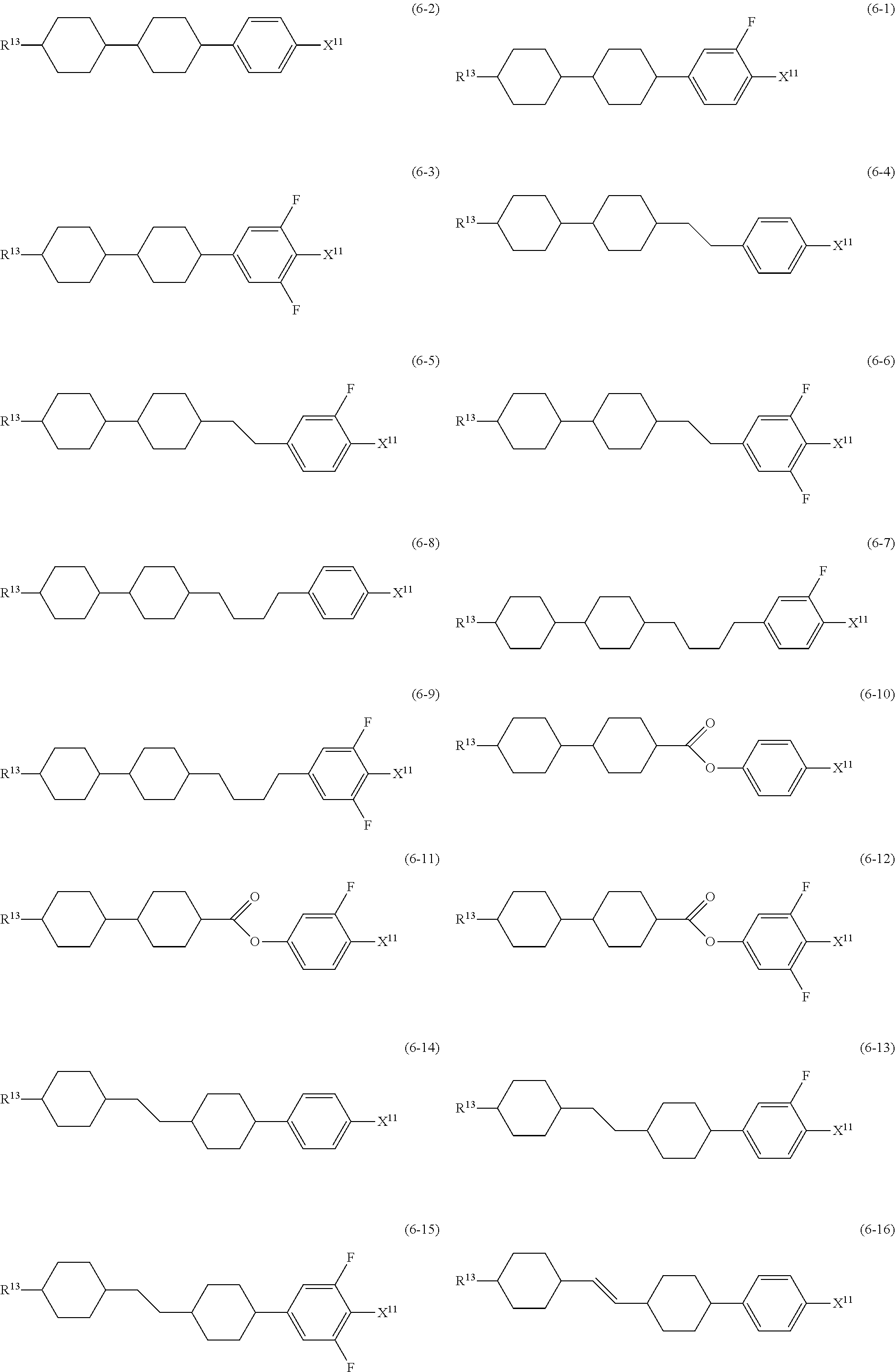

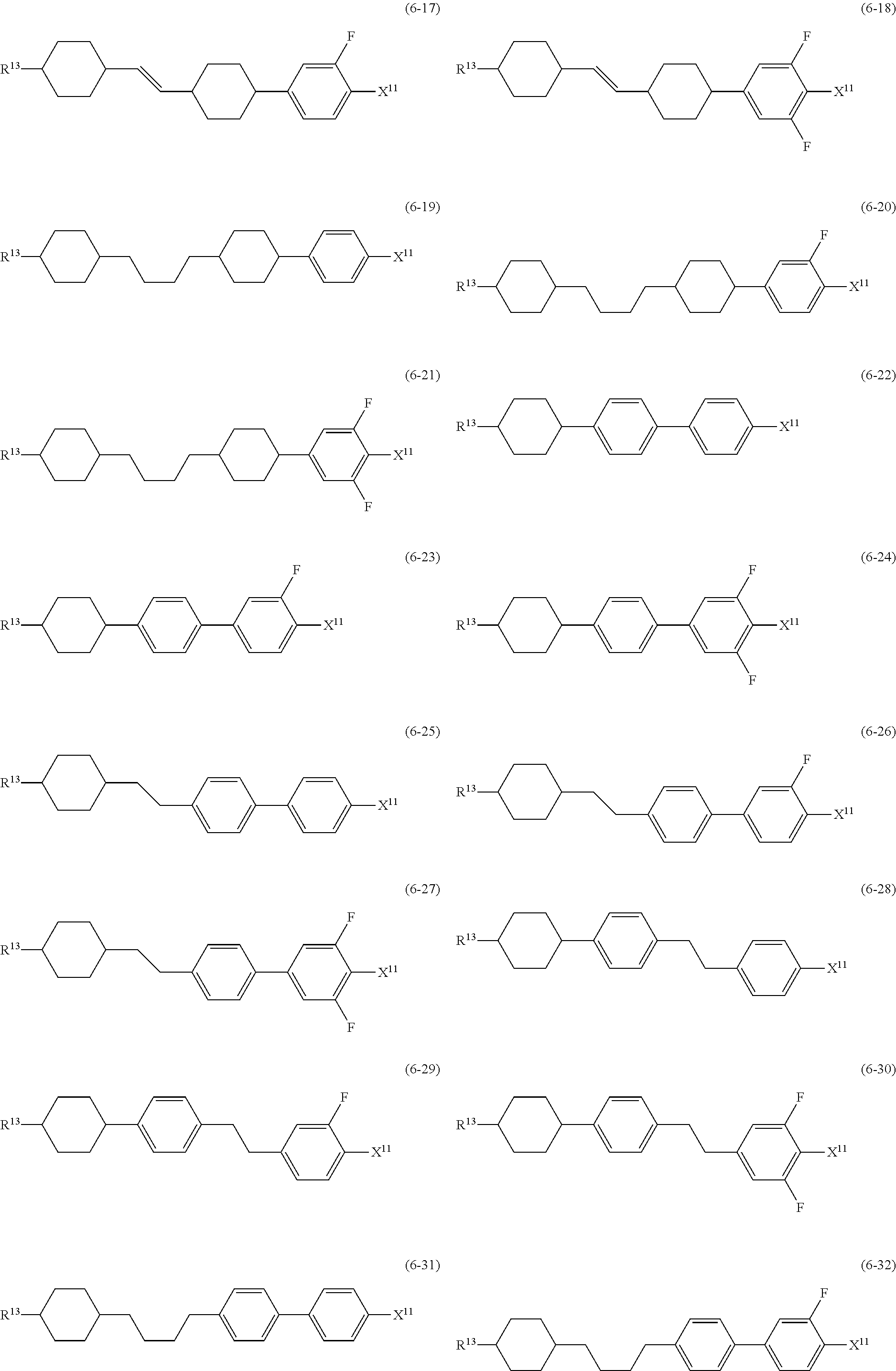

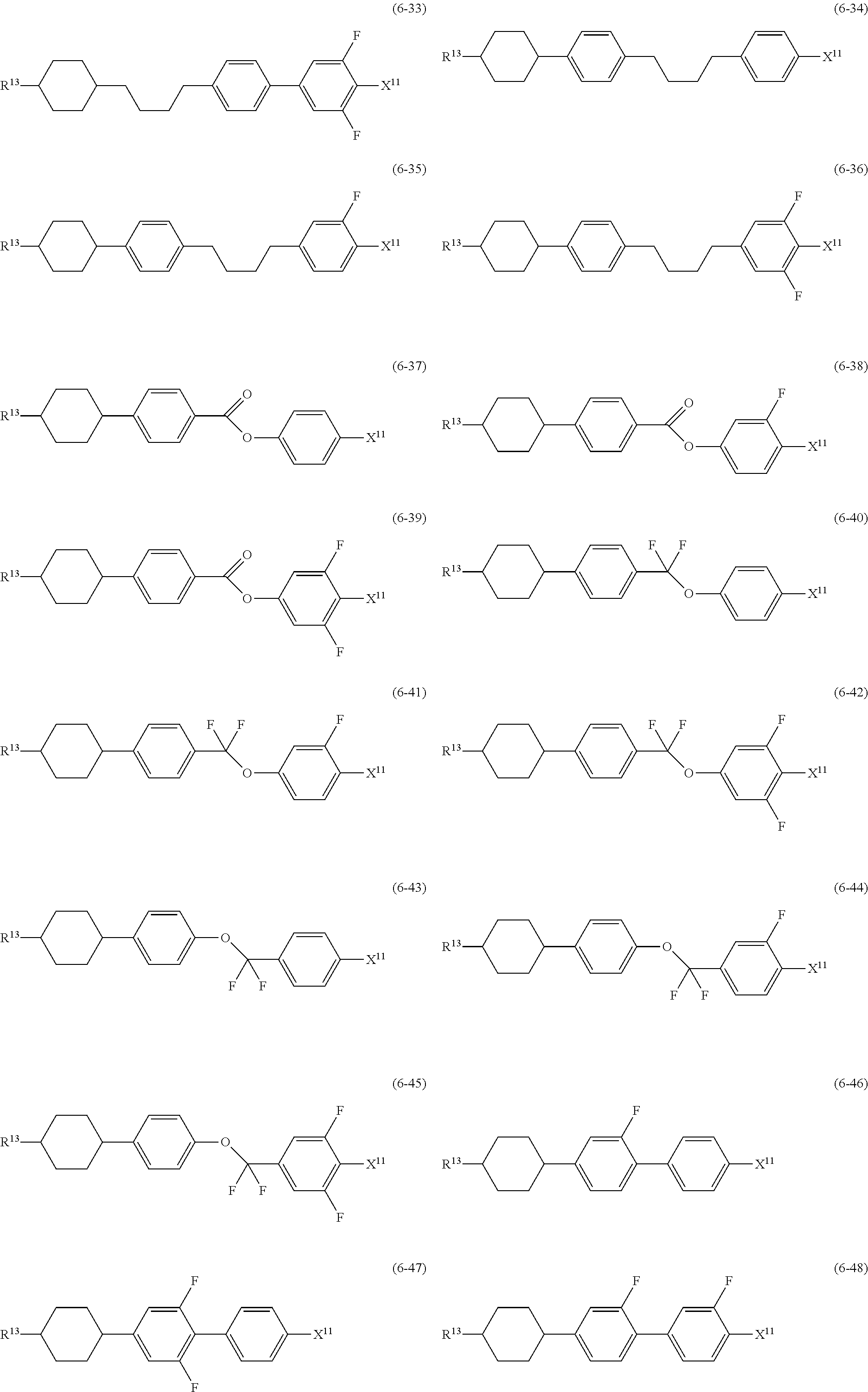

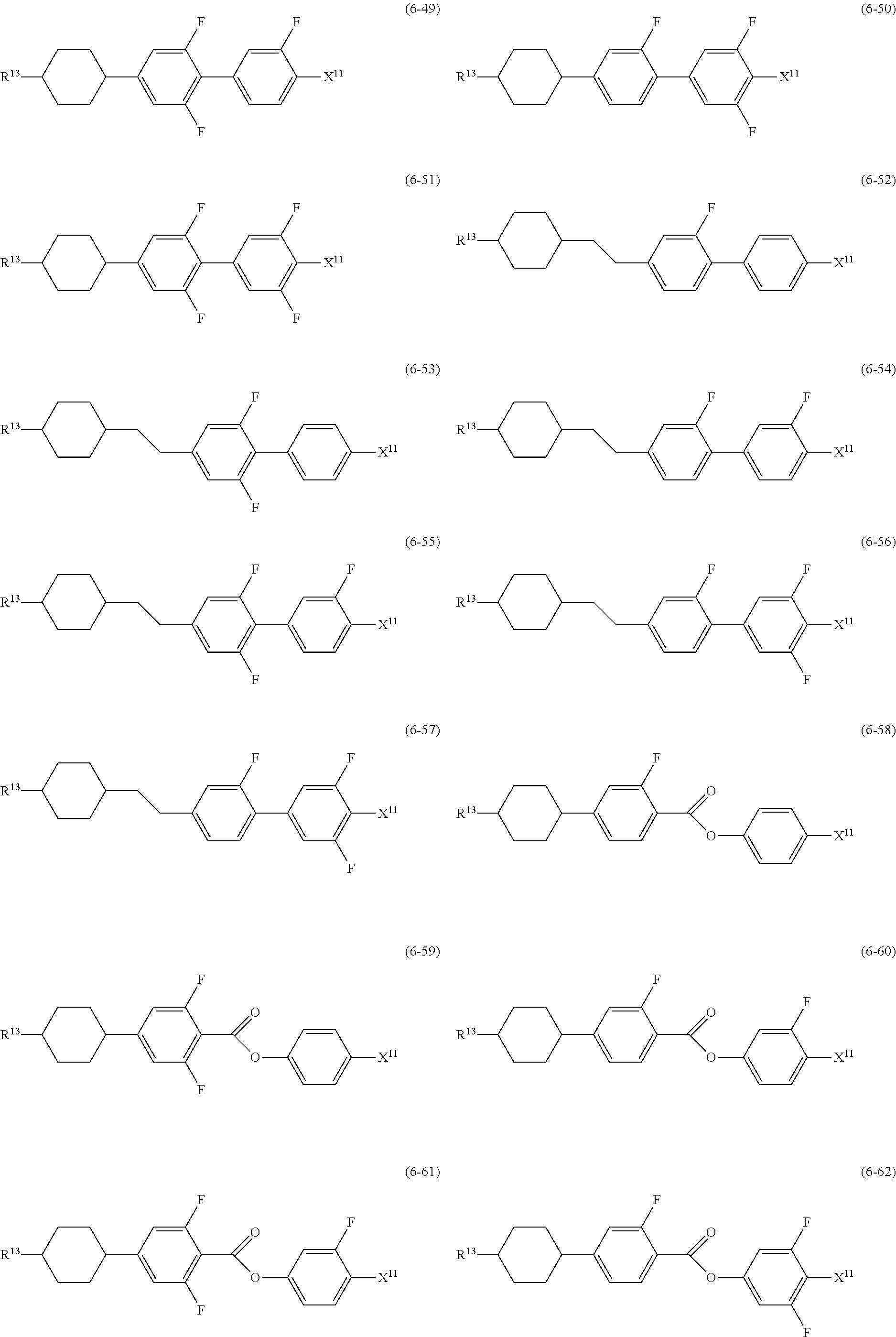

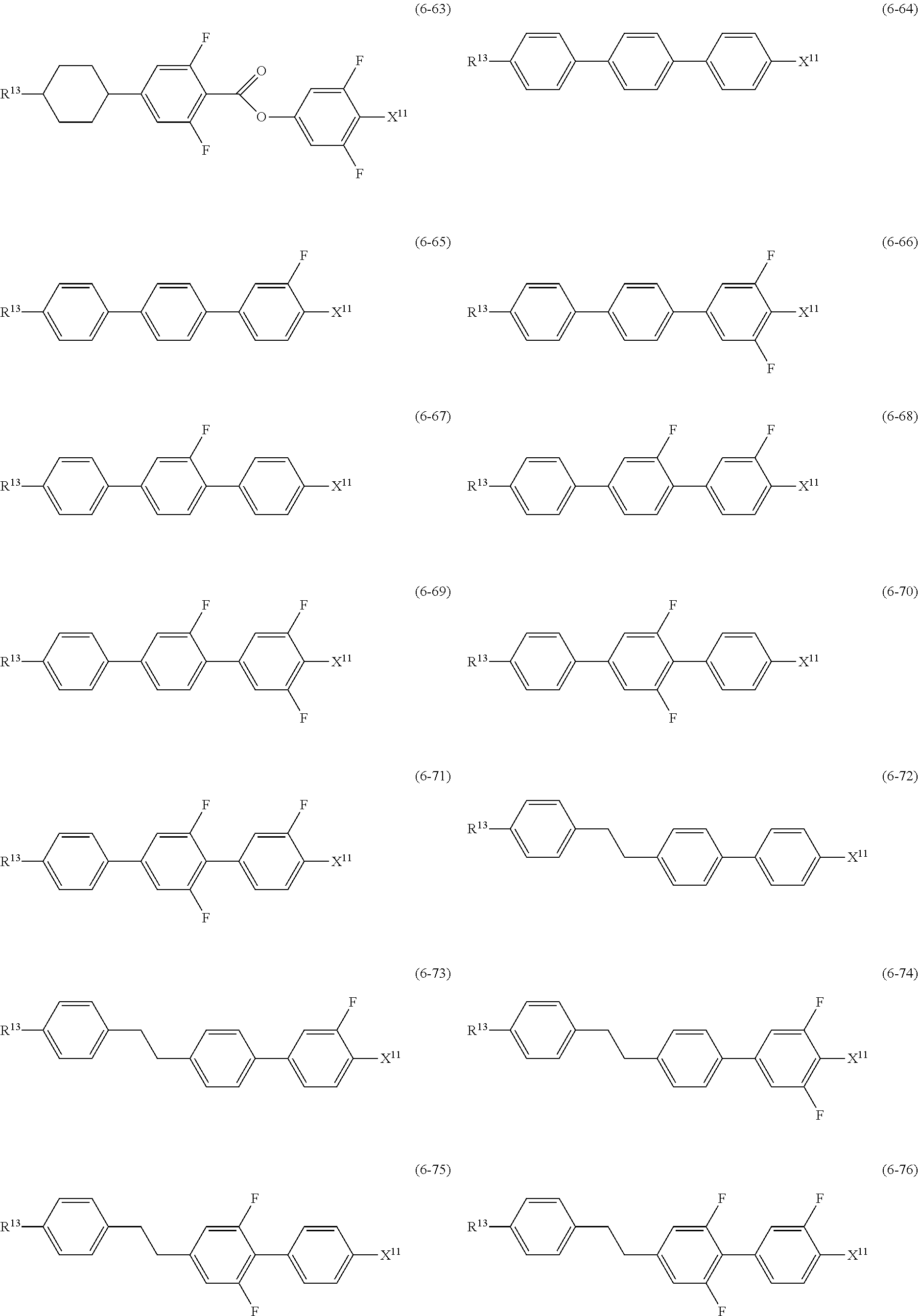

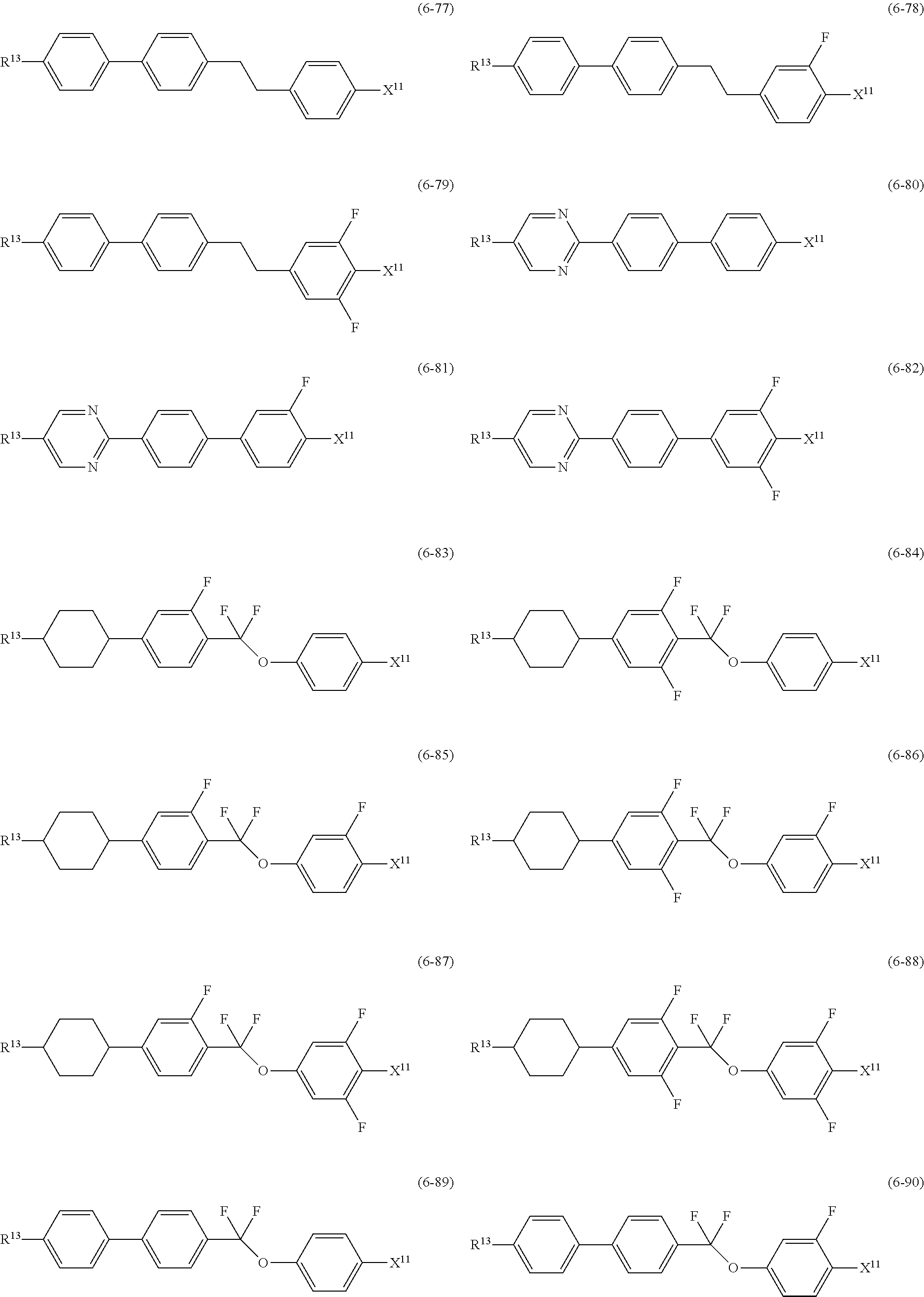

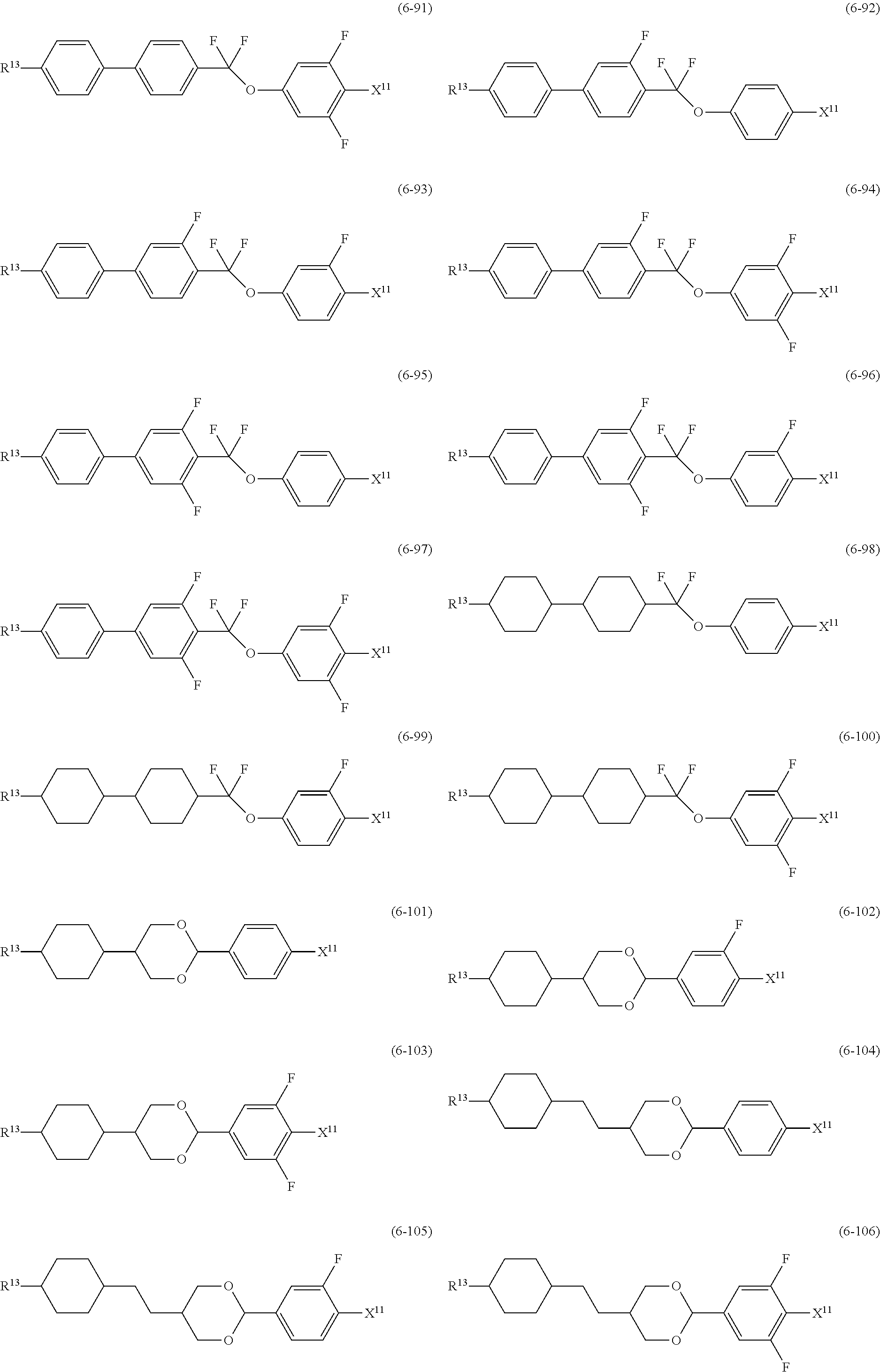

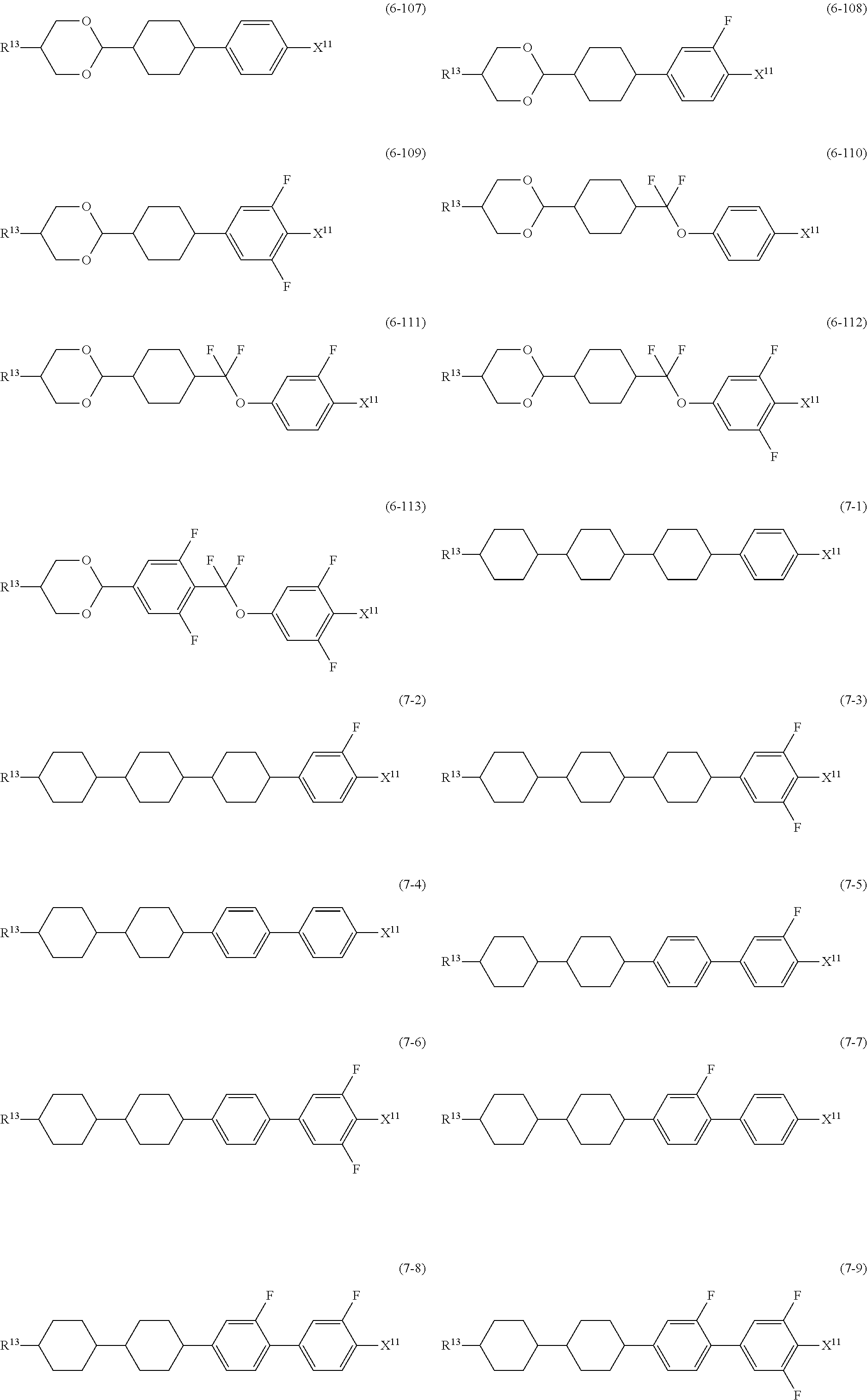

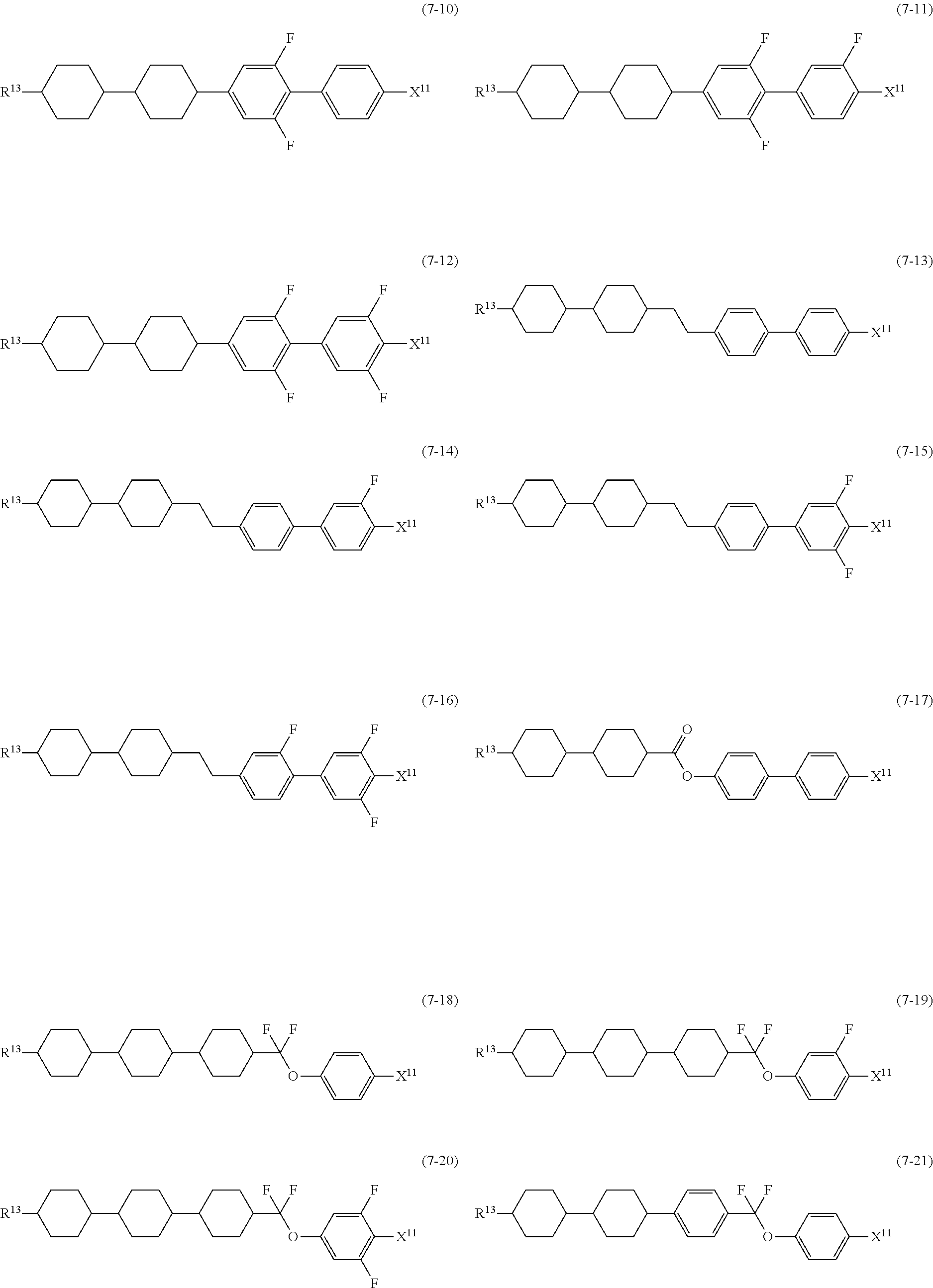

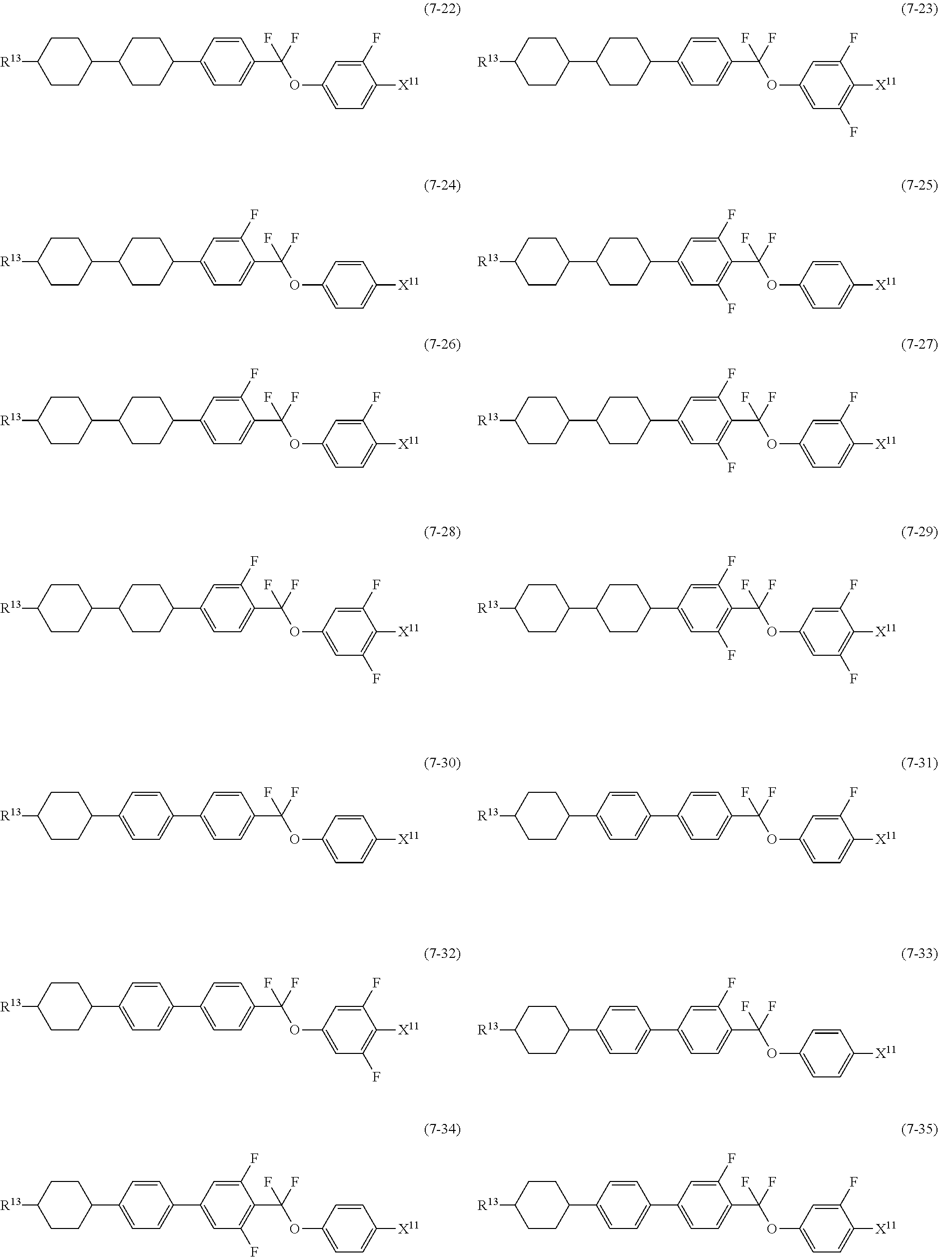

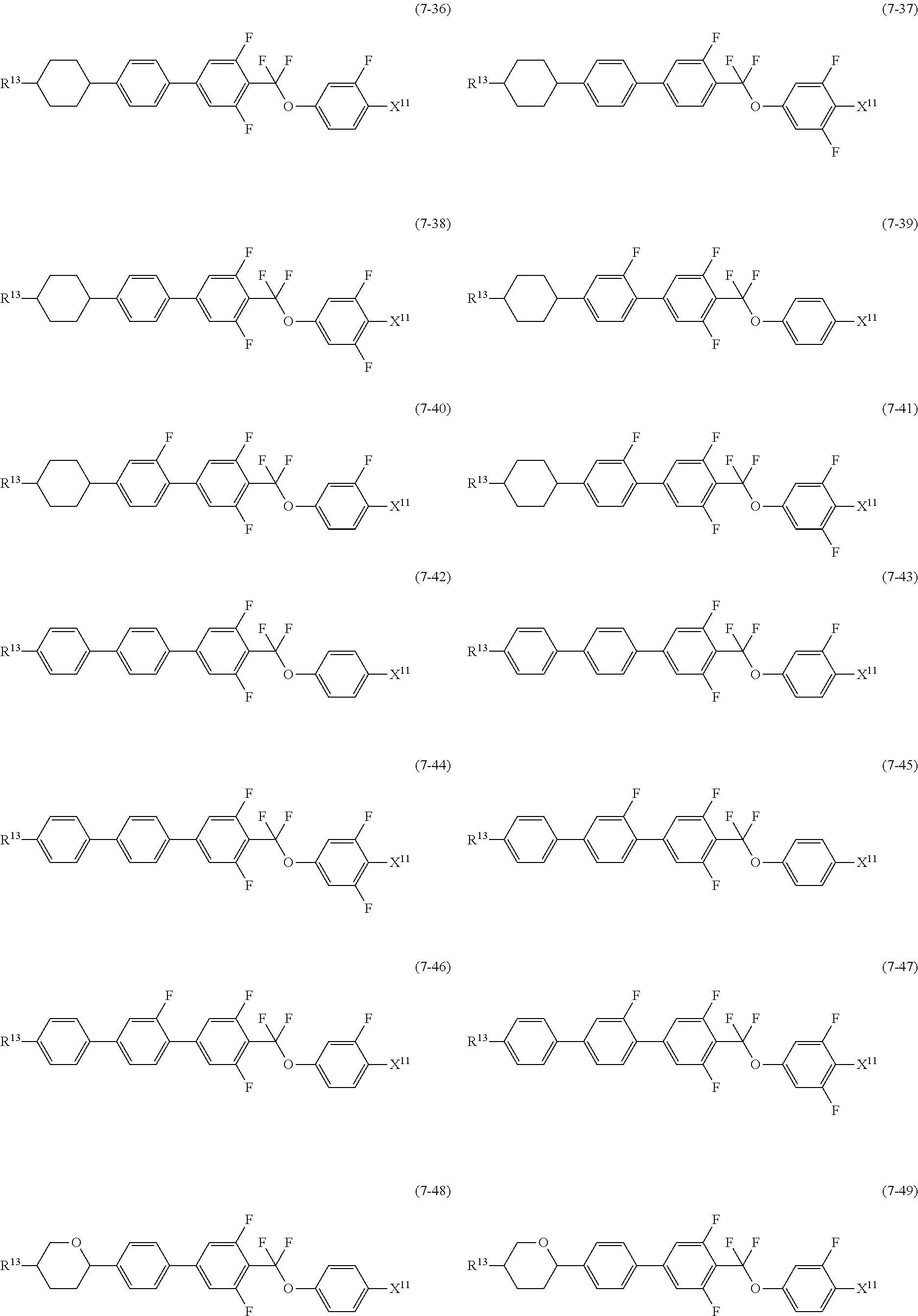

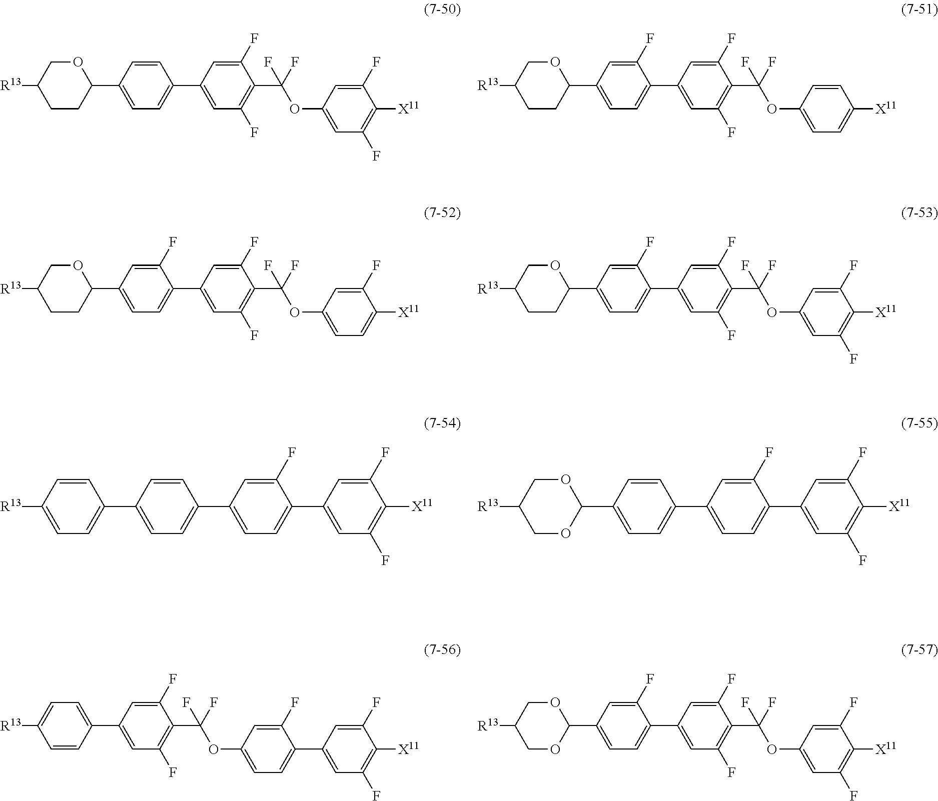

Component C has positive dielectric anisotropy and significantly good stability to heat or light, and therefore is used when a composition for the IPS mode, an FFS mode, an OCB mode or the like is prepared. A proportion of component C is suitably in the range of about 1% by weight to about 99% by weight, preferably in the range of about 10% by weight to about 97% by weight, and further preferably in the range of about 40% by weight to about 95% by weight, based thereon. When component C is added to a composition having negative dielectric anisotropy, the proportion of component C is preferably about 30% by weight or less based thereon. Addition of component C allows adjustment of the elastic constant of the composition and adjustment of a voltage-transmittance curve of the device.

Component D is compound (8) in which a right-terminal group is —C≡N or —C≡C—C≡N. Specific examples of preferred component D include compounds (8-1) to (8-64). In the compounds, R¹⁴ is alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine. X¹² is —C≡N or —C≡C—C≡N.

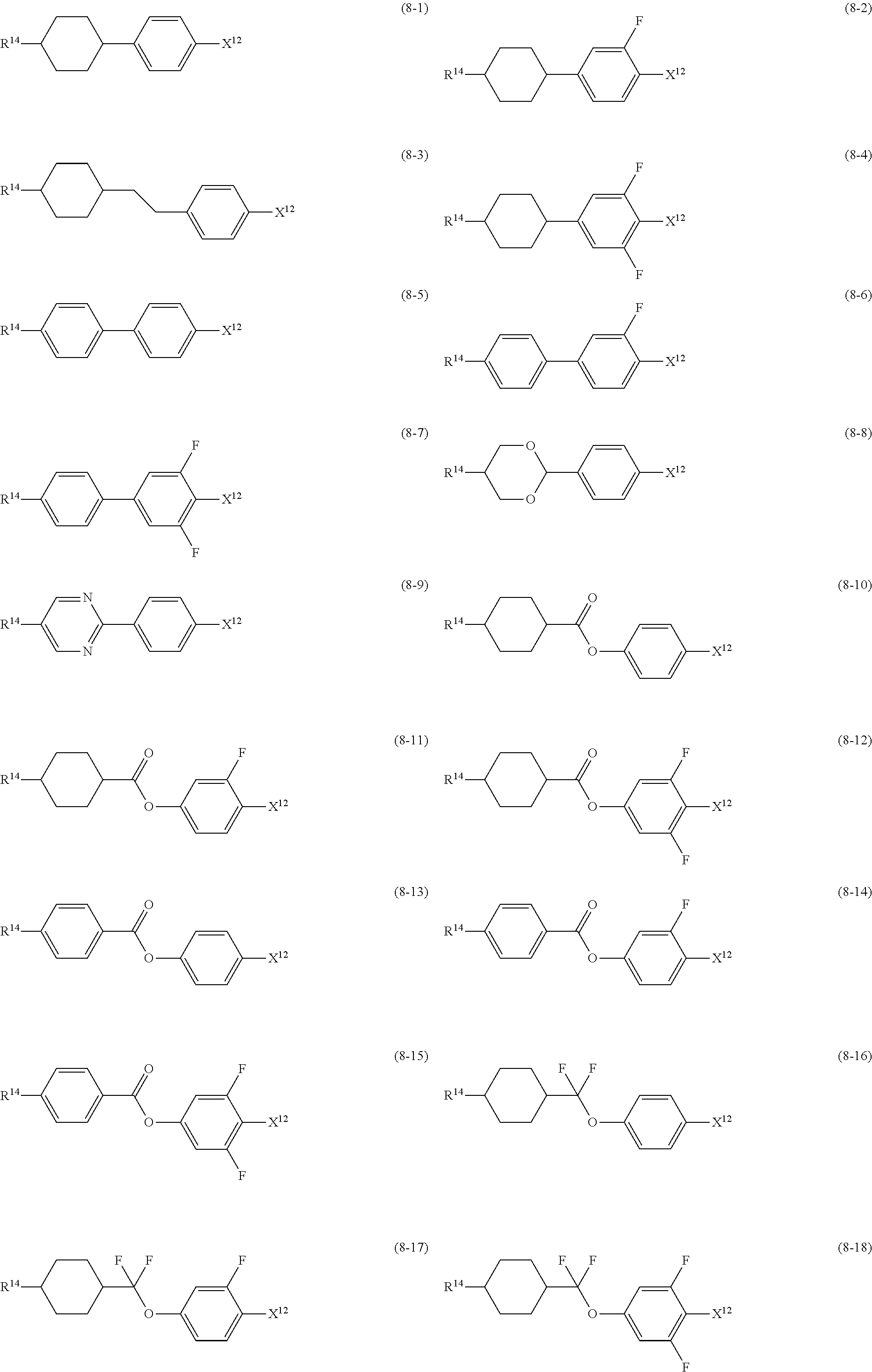

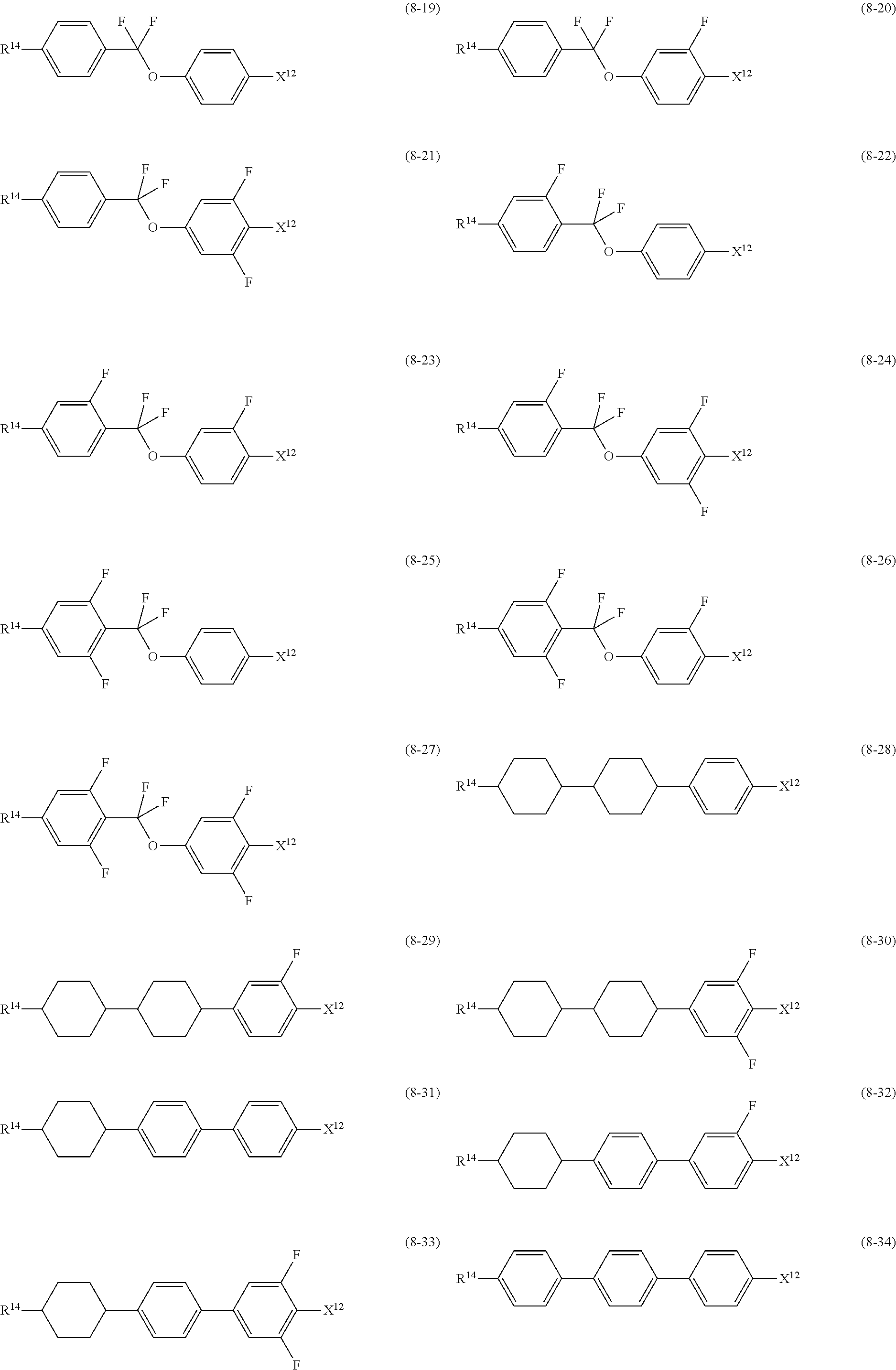

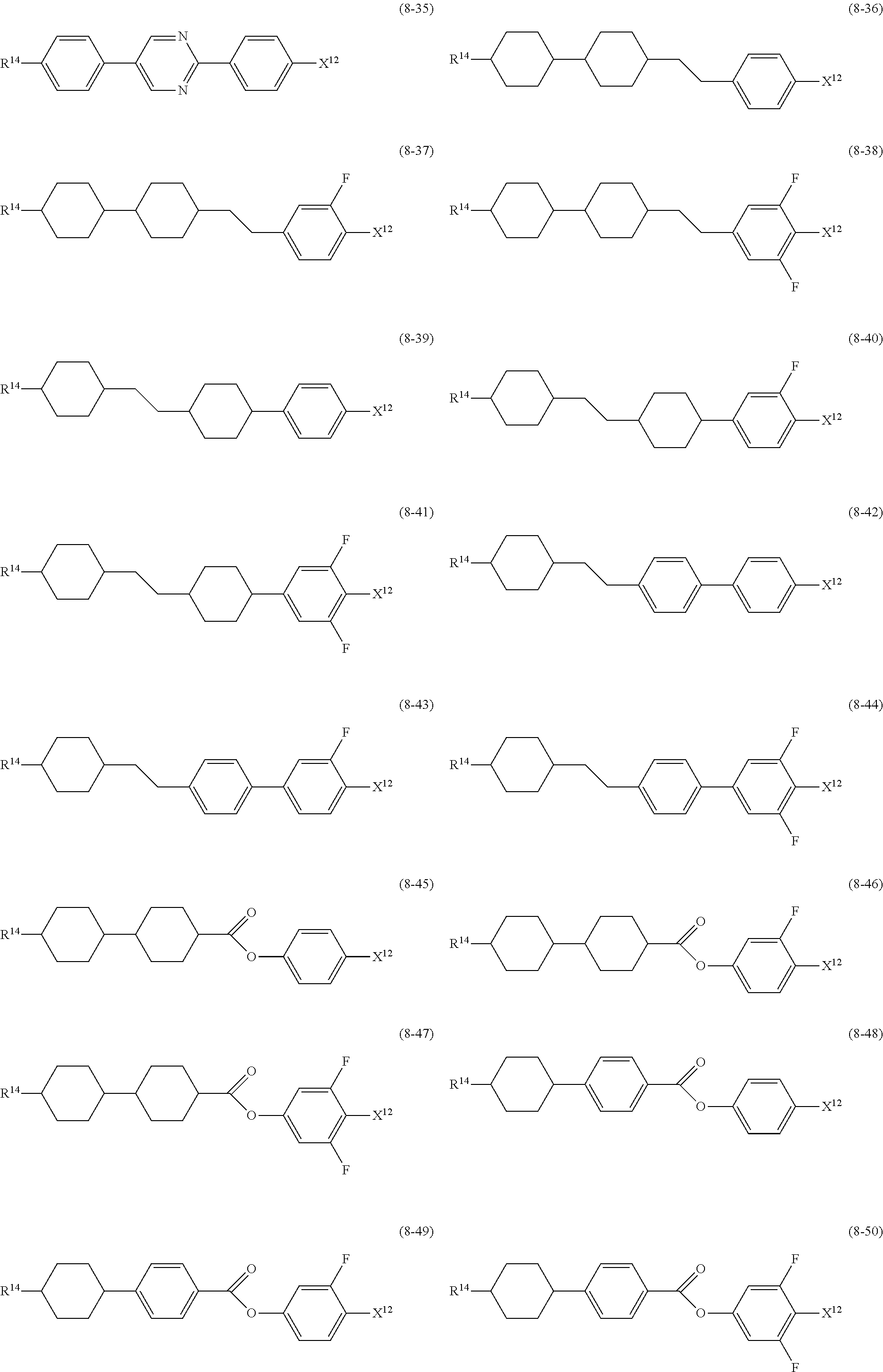

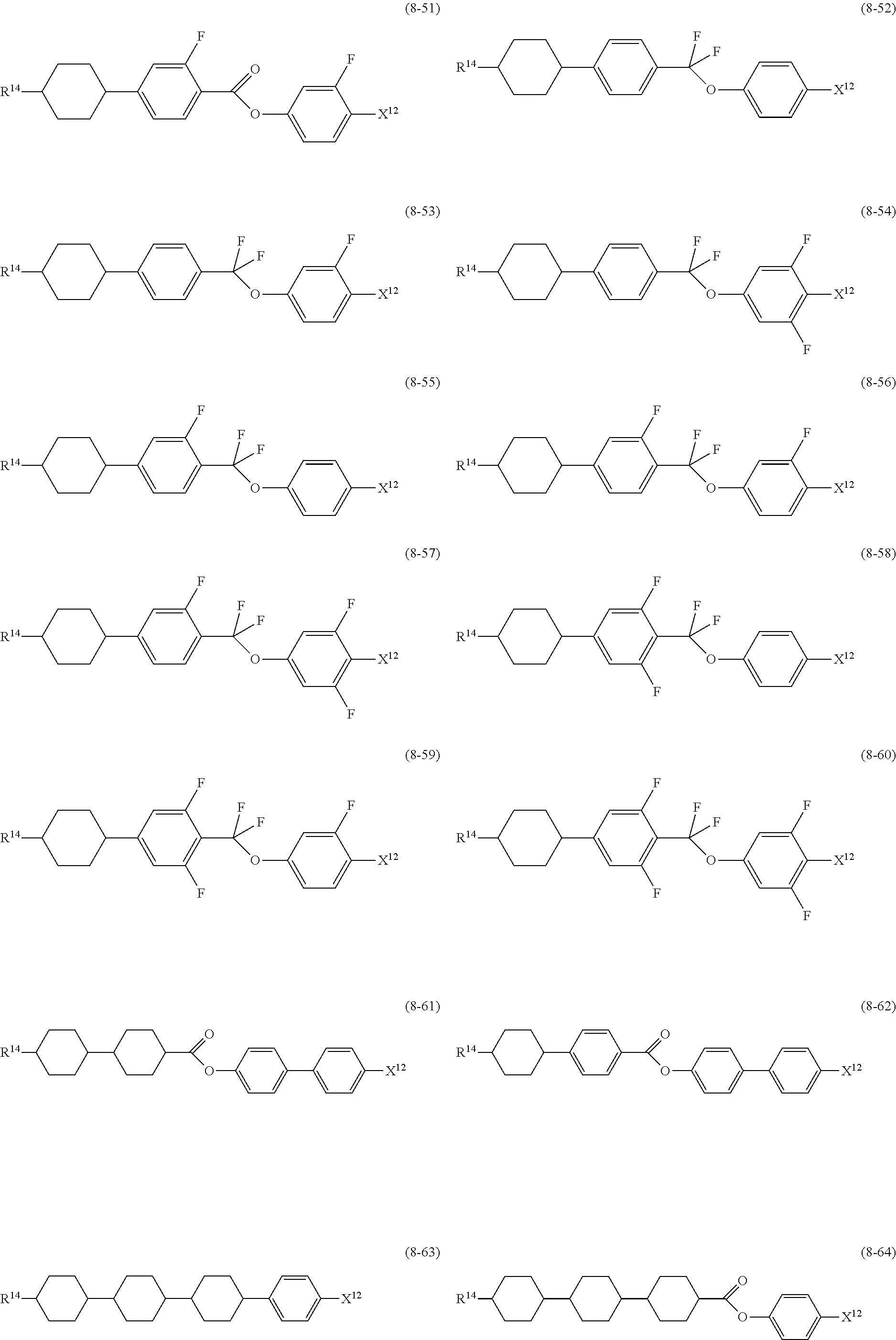

Component D has positive dielectric anisotropy and a value thereof is large, and therefore is used when a composition for a TN mode or the like is prepared. Addition of component D can increase the dielectric anisotropy of the composition. Component D is effective in extending the temperature range of the liquid crystal phase, adjusting the viscosity or adjusting the optical anisotropy. Component D is also useful for adjustment of the voltage-transmittance curve of the device.

When the composition for the TN mode or the like is prepared, a proportion of component D is suitably in the range of about 1% by weight to about 99% by weight, preferably in the range of about 10% by weight to about 97% by weight, and further preferably in the range of about 40% by weight to about 95% by weight, based thereon. When component D is added to the composition having negative dielectric anisotropy, the proportion of component D is preferably about 30% by weight or less based thereon. Addition of component D allows adjustment of the elastic constant of the composition and adjustment of the voltage-transmittance curve of the device.

Component E includes compounds (9) to (15). The compounds have phenylene in which hydrogen in lateral positions are replaced by two halogens, such as 2,3-difluoro-1,4-phenylene. Specific examples of preferred component E include compounds (9-1) to (9-8), compounds (10-1) to (10-17), compound (11-1), compounds (12-1) to (12-3), compounds (13-1) to (13-11), compounds (14-1) to (14-3) and compounds (15-1) to (15-3). In the compounds, R¹⁵, R¹⁶ and R¹⁷ are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine, and R¹⁷ may be hydrogen or fluorine.

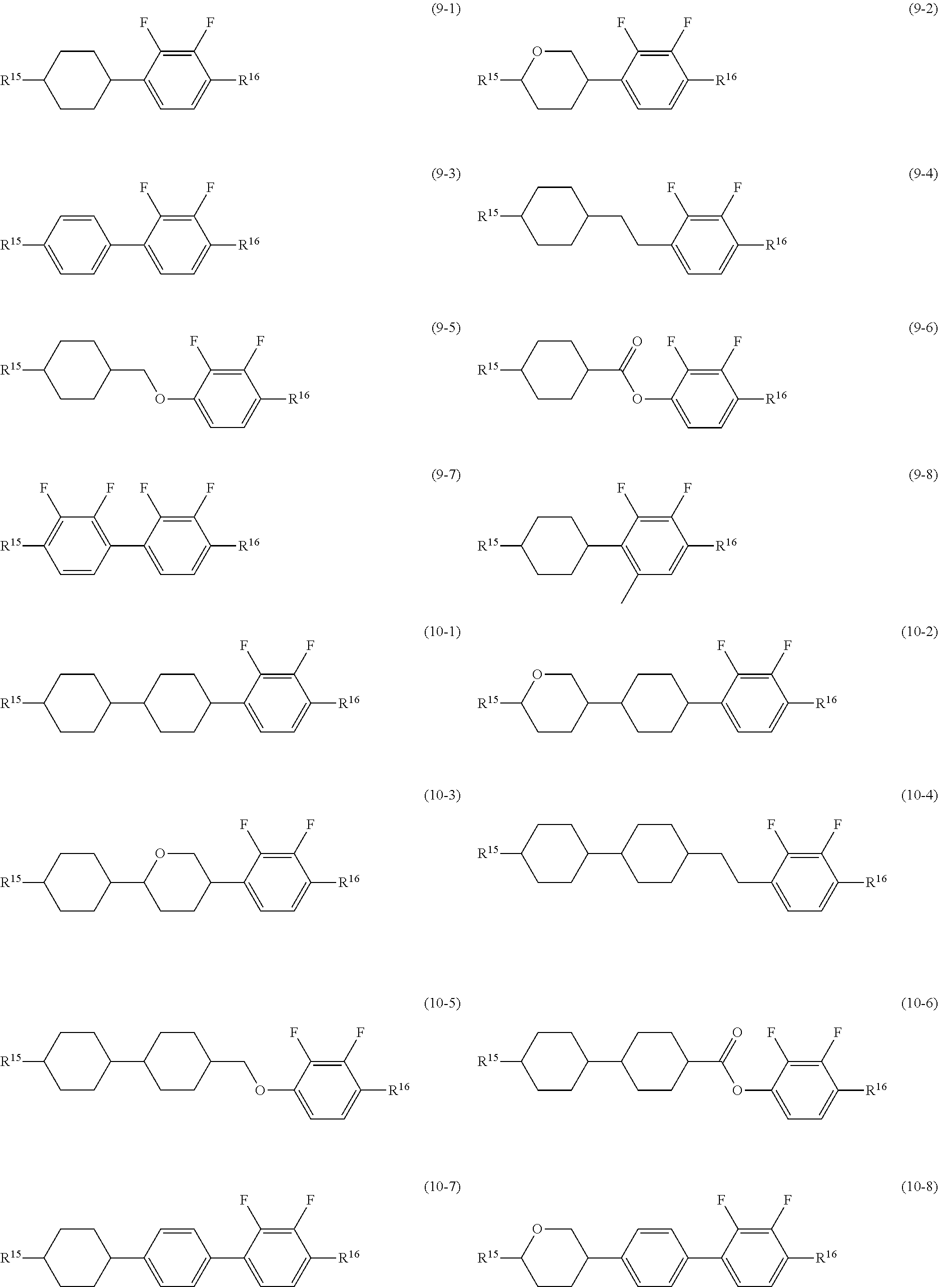

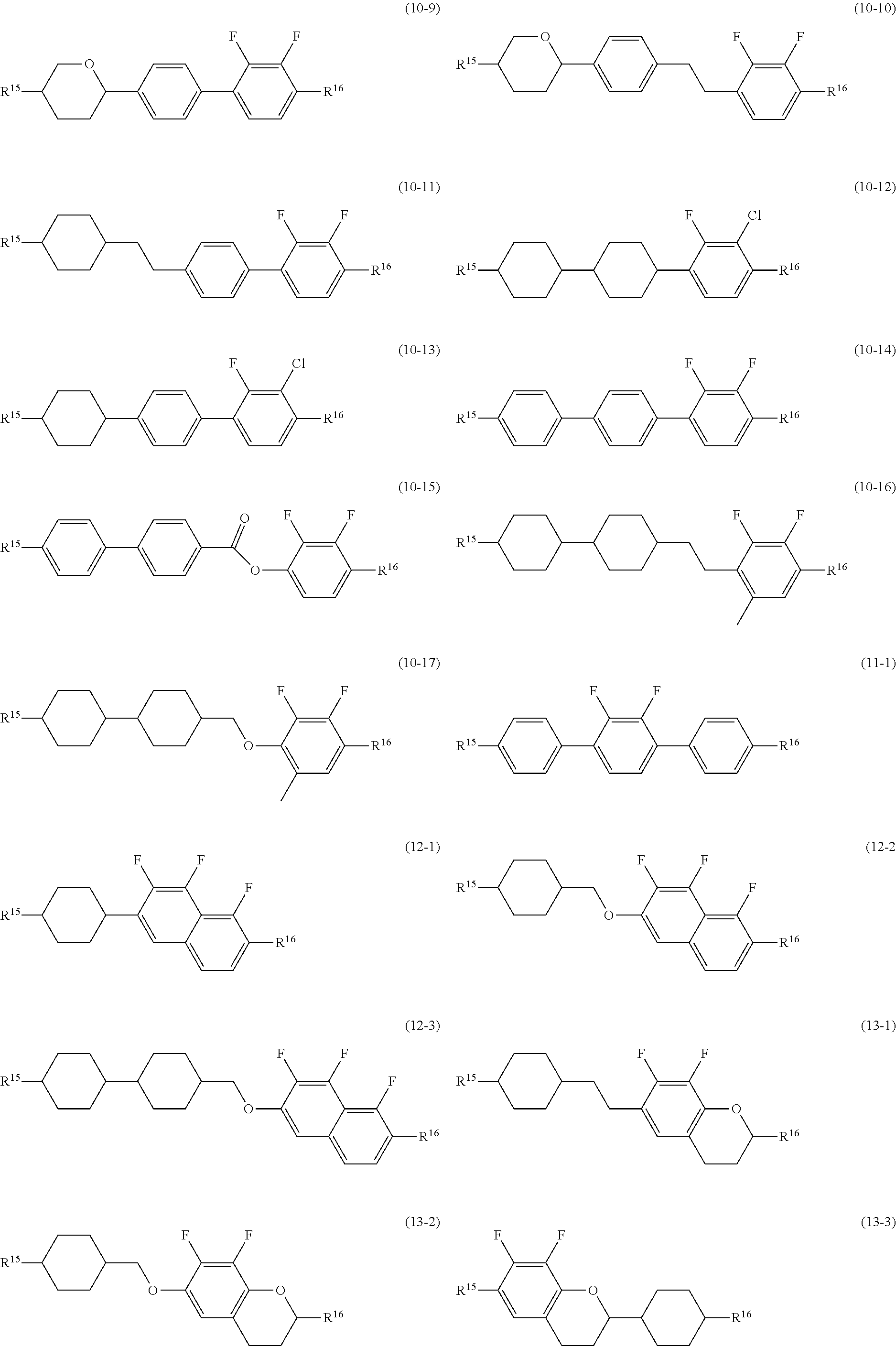

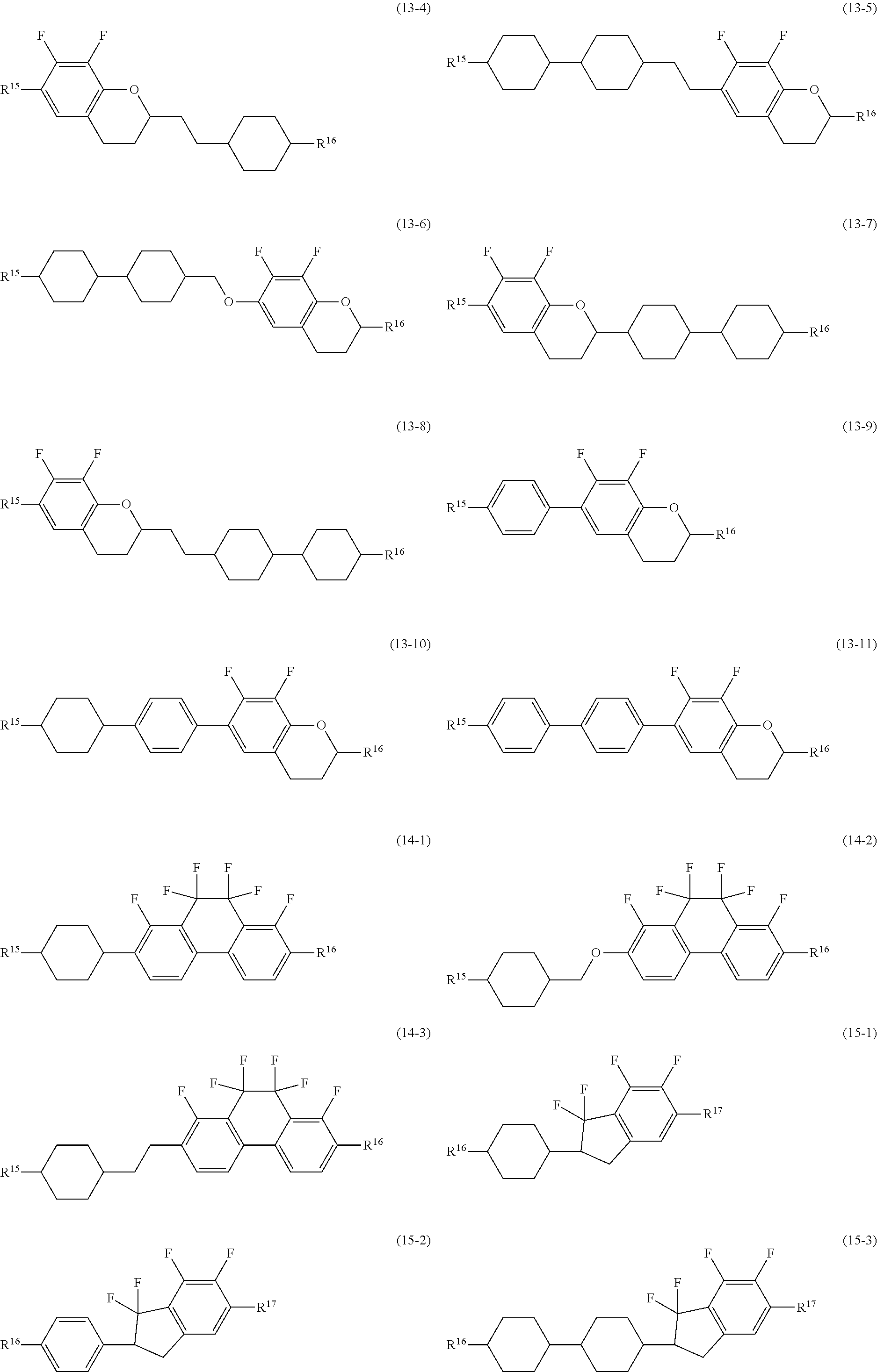

Component E has large negative dielectric anisotropy. Component E is used when a composition for the IPS mode, the VA mode, the PSA mode or the like is prepared. As a proportion of component E is increased, the dielectric anisotropy of the composition is negatively increased, but the viscosity is increased. Thus, as long as a desired value of threshold voltage of the device is met, the proportion is preferably as small as possible. When the dielectric anisotropy at a degree of −5 is taken into account, the proportion is preferably about 40% by weight or more in order to allow sufficient voltage driving, based thereon.

Among types of component E, compound (9) is a bicyclic compound, and therefore is effective in decreasing the viscosity, adjusting the optical anisotropy or increasing the dielectric anisotropy. Compounds (10) and (11) are a tricyclic compound, and therefore are effective in increasing the maximum temperature, the optical anisotropy or the dielectric anisotropy. Compounds (12) to (15) are effective in increasing the dielectric anisotropy.

When the composition for the IPS mode, the VA mode, the PSA mode or the like is prepared, the proportion of component E is preferably about 40% by weight or more, and further preferably in the range of about 50% by weight to about 95% by weight, based thereon. When component E is added to a composition having positive dielectric anisotropy, the proportion of component E is preferably about 30% by weight or less based thereon. Addition of component E allows adjustment of the elastic constant of the composition and adjustment of the voltage-transmittance curve of the device.

A liquid crystal composition satisfying at least one of physical properties such as high stability to heat or light, high maximum temperature, low minimum temperature, small viscosity, suitable optical anisotropy (more specifically, large optical anisotropy or small optical anisotropy), large positive or negative dielectric anisotropy, large specific resistance and a suitable elastic constant (more specifically, a large elastic constant or a small elastic constant) can be prepared by suitably combining components B, C, D and E with compound (1). A device including such a composition has a wide temperature range in which the device can be used, a short response time, a large voltage holding ratio, low threshold voltage, a large contrast ratio, a small flicker rate and a long service life.

If the device is used for a long period of time, a flicker may be occasionally generated on a display screen. The flicker rate (%) can be represented by a formula (|luminance when applying positive voltage−luminance when applying negative voltage|)/(average luminance)×100. In a device having the flicker rate in the range of about 0% to about 1%, a flicker is hardly generated on the display screen even if the device is used for a long period of time. The flicker is associated with image persistence, and is presumed to be generated according to a difference in electric potential between a positive frame and a negative frame in driving at alternating current. The composition containing compound (1) is also useful for reducing generation of the flicker.

### 3-2. Additive

A liquid crystal composition is prepared according to a publicly known method. For example, the component compounds are mixed and dissolved in each other by heating. According to an application, an additive may be added to the composition. Examples of the additive include the polymerizable compound, the polymerization initiator, the polymerization inhibitor, the optically active compound, the antioxidant, the ultraviolet light absorber, the light stabilizer, the heat stabilizer, the dye and the antifoaming agent. Such an additive is well known to those skilled in the art, and described in literature.

In the liquid crystal display device having the polymer sustained alignment (PSA) mode, the composition contains a polymer. The polymerizable compound is added for the purpose of forming the polymer in the composition. The polymerizable compound is polymerized by irradiation with ultraviolet light while voltage is applied between electrodes, and thus the polymer is formed in the composition. A suitable pretilt is achieved by the method, and therefore the device in which a response time is shortened and the image persistence is improved is prepared.

Preferred examples of the polymerizable compound include acrylate, methacrylate, a vinyl compound, a vinyloxy compound, propenyl ether, an epoxy compound (oxirane, oxetane) and vinyl ketone. Further preferred examples include a compound having at least one acryloyloxy, and a compound having at least one methacryloyloxy. Still further preferred examples also include a compound having both acryloyloxy and methacryloyloxy.

Still further preferred examples include compounds (M-1) to (M-18). In the compounds, R²⁵ to R³¹ are independently hydrogen or methyl; R³², R³³ and R³⁴ are independently hydrogen or alkyl having 1 to 5 carbons, and at least one of R³², R³³ and R³⁴ is alkyl having 1 to 5 carbons; v, w and x are independently 0 or 1; and u and v are independently an integer from 1 to 10. L²¹ to L²⁶ are independently hydrogen or fluorine; and L²⁷ and L²⁸ are independently hydrogen, fluorine or methyl.

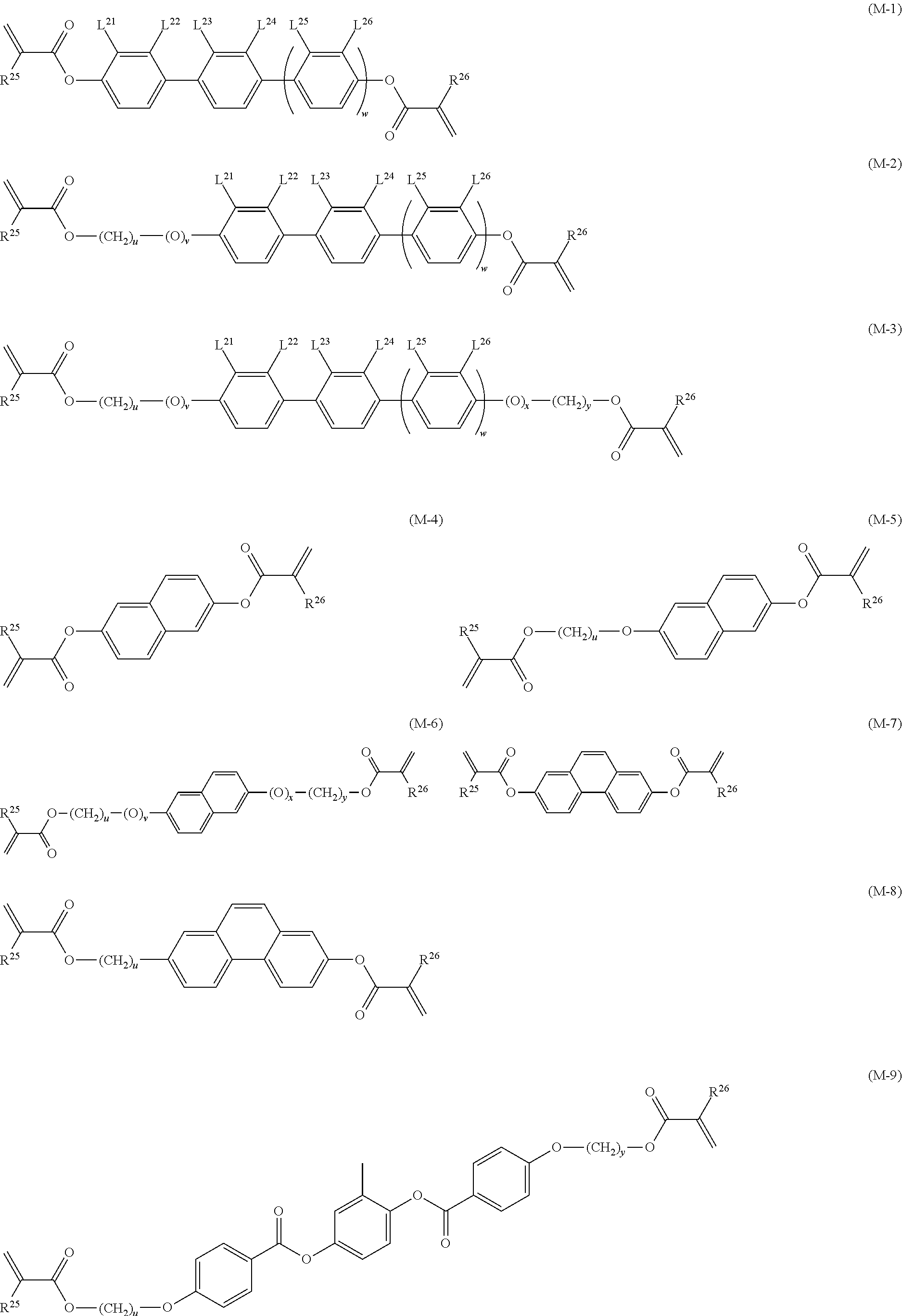

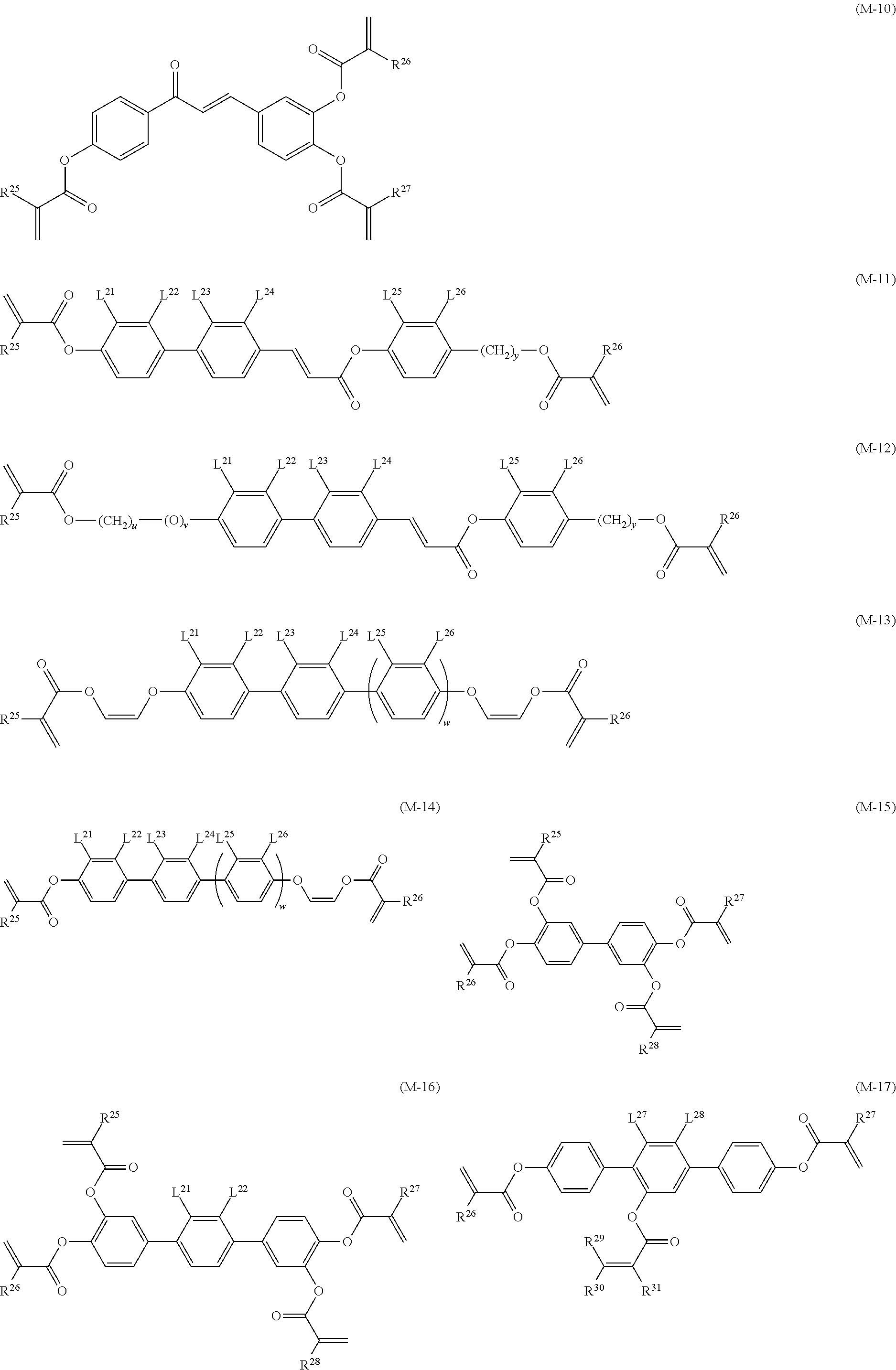

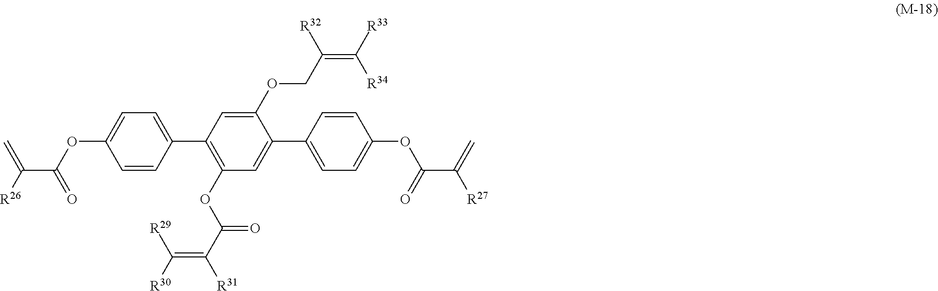

The polymerizable compound can be rapidly polymerized by adding the polymerization initiator. An amount of a remaining polymerizable compound can be reduced by optimizing reaction conditions. Examples of a photoradical polymerization initiator include TPO, 1173 and 4265 from Darocur series of BASF SE, and 184, 369, 500, 651, 784, 819, 907, 1300, 1700, 1800, 1850 and 2959 from Irgacure series thereof.

Additional examples of the photoradical polymerization initiator include 4-methoxyphenyl-2,4-bis(trichloromethyl)triazine, 2-(4-butoxystyryl)-5-trichloromethyl-1,3,4-oxadiazole, 9-phenylacridine, 9,10-benzphenazine, a benzophenone-Michler's ketone mixture, a hexaarylbiimidazole-mercaptobenzimidazole mixture, 1-(4-isopropylphenyl)-2-hydroxy-2-methylpropane-1-one, benzyl dimethyl ketal, 2-methyl-1-[4-(methylthio)phenyl]-2-morpholinopropane-1-one, a mixture of 2,4-diethylxanthone and methyl p-dimethylaminobenzoate, and a mixture of benzophenone and methyltriethanolamine.

After the photoradical polymerization initiator is added to the liquid crystal composition, polymerization can be performed by irradiation with ultraviolet light while an electric field is applied. However, an unreacted polymerization initiator or a decomposition product of the polymerization initiator may cause poor display such as image persistence in the device. In order to prevent such an event, photopolymerization may be performed with no addition of the polymerization initiator. A preferred wavelength of irradiation light is in the range of about 150 nanometers to about 500 nanometers. A further preferred wavelength is in the range of about 250 nanometers to about 450 nanometers, and a most preferred wavelength is in the range of about 300 nanometers to about 400 nanometers.

Upon storing the polymerizable compound, the polymerization inhibitor may be added thereto for preventing polymerization. The polymerizable compound is ordinarily added to the composition without removing the polymerization inhibitor. Examples of the polymerization inhibitor include hydroquinone, a hydroquinone derivative such as methylhydroquinone, 4-t-butylcatechol, 4-methoxyphenol and phenothiazine.

The optically active compound is effective in inducing helical structure in liquid crystal molecules to give a required twist angle, and thereby preventing a reverse twist. A helical pitch can be adjusted by adding the optically active compound thereto. Two or more optically active compounds may be added for the purpose of adjusting temperature dependence of the helical pitch. Specific examples of a preferred optically active compound include compounds (Op-1) to (Op-18) described below. In compound (Op-18), ring J is 1,4-cyclohexylene or 1,4-phenylene, and R²⁸ is alkyl having 1 to 10 carbons. Asterisk mark (*) represents asymmetrical carbon.

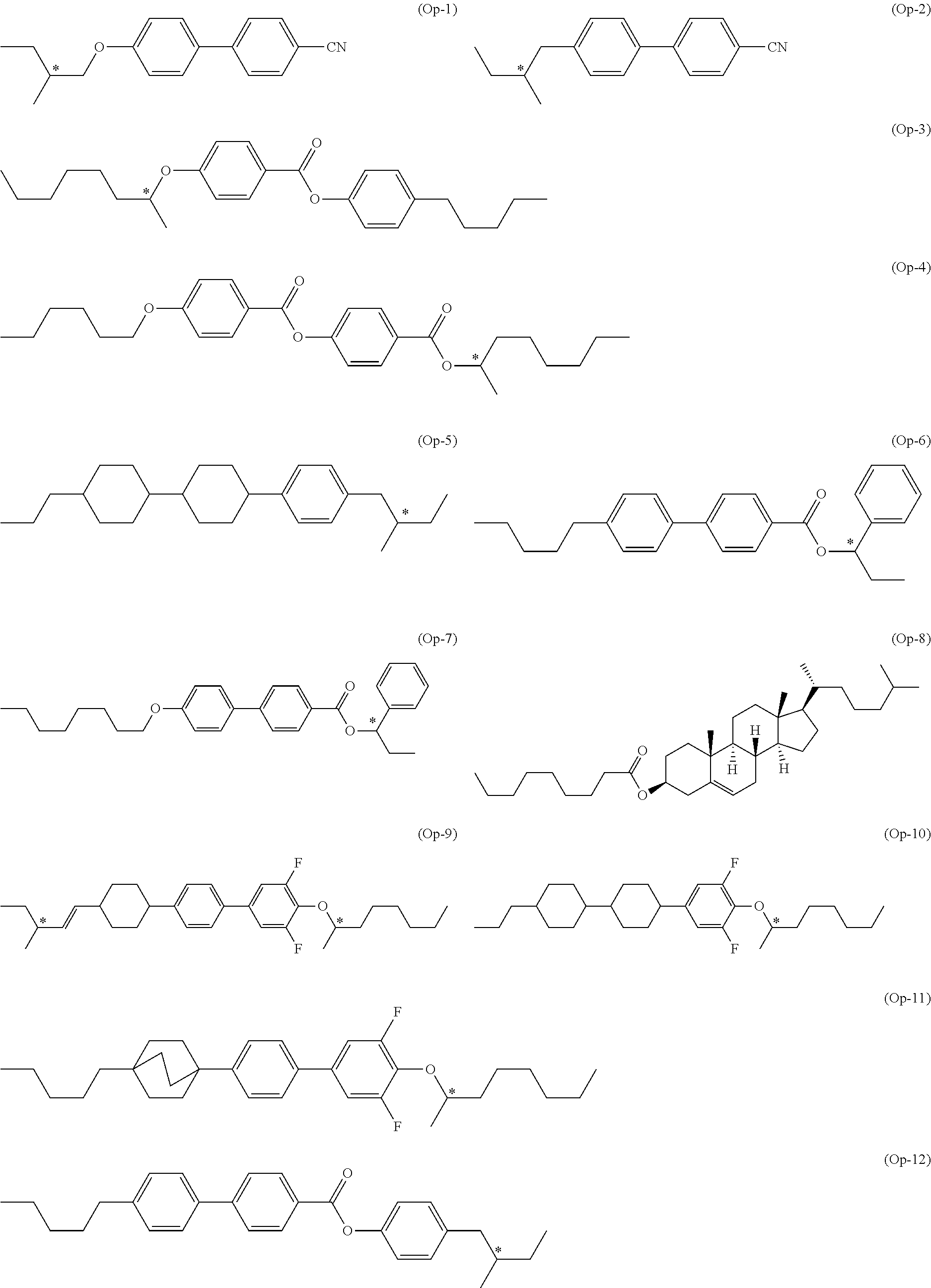

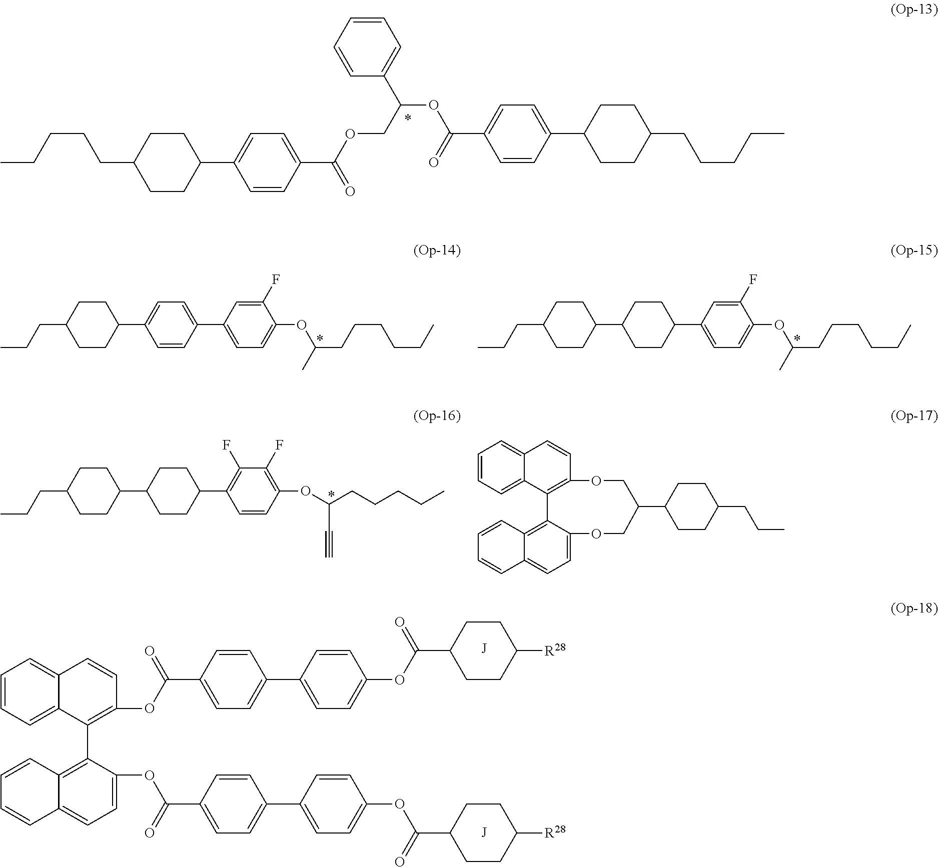

The antioxidant is effective for maintaining the large voltage holding ratio. Specific examples of a preferred antioxidant include compounds (AO-1) and (AO-2) described below; and Irganox 415, Irganox 565, Irganox 1010, Irganox 1035, Irganox 3114 and Irganox 1098 (trade names; BASF SE). The ultraviolet light absorber is effective for preventing a decrease of the maximum temperature. Preferred examples of the ultraviolet light absorber include a benzophenone derivative, a benzoate derivative and a triazole derivative, and specific examples include compounds (AO-3) and (AO-4) described below; Tinuvin 329, Tinuvin P, Tinuvin 326, Tinuvin 234, Tinuvin 213, Tinuvin 400, Tinuvin 328 and Tinuvin 99-2 (trade names; BASF SE); and 1,4-diazabicyclo[2.2.2]octane (DABCO).

The light stabilizer such as an amine having steric hindrance is preferred for maintaining the large voltage holding ratio. Specific examples of a preferred light stabilizer include compounds (AO-5), (AO-6) and (AO-7) described below; Tinuvin 144, Tinuvin 765 and Tinuvin 770DF (trade names; BASF SE); and LA-77Y and LA-77G (trade names; ADEKA Corporation). The heat stabilizer is also effective for maintaining the large voltage holding ratio, and specific preferred examples include Irgafos 168 (trade name; BASF SE). A dichroic dye such as an azo dye or an anthraquinone dye is added to the composition to be adapted for a device having a guest host (GH) mode. The antifoaming agent is effective for preventing foam formation. Preferred examples of the antifoaming agent include dimethyl silicone oil and methylphenyl silicone oil.

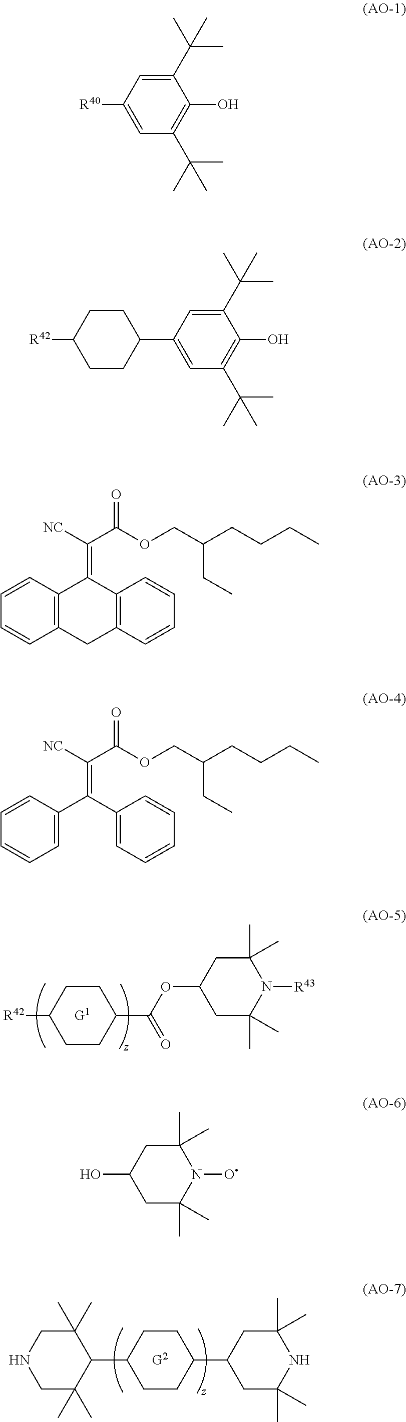

In compound (AO-1), R⁴⁰ is alkyl having 1 to 20 carbons, alkoxy having 1 to 20 carbons, —COOR⁴¹ or —CH₂CH₂COOR⁴¹, in which R⁴¹ is alkyl having 1 to 20 carbons. In compounds (AO-2) and (AO-5), R⁴² is alkyl having 1 to 20 carbons. In compound (AO-5), R⁴³ is hydrogen, methyl or O. (oxygen radical); ring G¹ is 1,4-cyclohexylene or 1,4-phenylene; in compound (AO-7), ring G² is 1,4-cyclohexylene, 1,4-phenylene, or 1,4-phenylene in which at least one hydrogen is replaced by fluorine; and in compounds (AO-5) and (AO-7), z is 1, 2 or 3.

### 4. Liquid Crystal Display Device

The liquid crystal composition can be used in a liquid crystal display device having an operating mode such as the PC mode, the TN mode, the STN mode, the OCB mode and the PSA mode, and driven by an active matrix mode. The composition can also be used in a liquid crystal display device having the operating mode such as the PC mode, the TN mode, the STN mode, the OCB mode, the VA mode and the IPS mode, and driven by a passive matrix mode. The devices can be applied to any of a reflective type, a transmissive type and a transflective type.

The composition is also suitable for a nematic curvilinear aligned phase (NCAP) device, and the composition is microencapsulated herein. The composition can also be used in a polymer dispersed liquid crystal display device (PDLCD) or a polymer network liquid crystal display device (PNLCD). In the compositions, a large amount of polymerizable compound is added. On the other hand, when a proportion of the polymerizable compound is about 10% by weight or less based thereon, the liquid crystal display device having the PSA mode is prepared. A preferred proportion is in the range of about 0.1% by weight to about 2% by weight based thereon. A further preferred proportion is in the range of about 0.2% by weight to about 1.0% by weight based thereon. The device having the PSA mode can be driven by the driving mode such as the active matrix mode and the passive matrix mode. Such devices can be applied to any of the reflective type, the transmissive type and the transflective type.

### EXAMPLES

### 1. Example of Compound (1)

The invention will be described in greater detail by way of Examples. The Examples include a typical example, and therefore the invention is not limited by the Examples. Compound (1) was prepared according to procedures described below. The thus prepared compound was identified by methods such as an NMR analysis. Physical properties of the compound and the composition, and characteristics of a device were measured by methods described below.

NMR analysis: For measurement, DRX-500 made by Bruker BioSpin Corporation was used. In ¹H-NMR measurement, a sample was dissolved in a deuterated solvent such as CDCl₃, and measurement was carried out under conditions of room temperature, 500 MHz and times of accumulation. Tetramethylsilane was used as an internal standard. In ¹⁹F-NMR measurement, CFCl₃ was used as an internal standard, and measurement was carried out under conditions of 24 times of accumulation. In explaining nuclear magnetic resonance spectra obtained, s, d, t, q, quin, sex and m stand for a singlet, a doublet, a triplet, a quartet, a quintet, a sextet and a multiplet, and br being broad, respectively.

Mass analysis: For measurement, QP-2010 Ultra Gas Chromatograph Mass Spectrometer made by Shimadzu Corporation was used. As a column, a capillary column DB-1 (length 60 m, bore 0.25 mm, film thickness 0.25 μm) made by Agilent Technologies, Inc. was used. As a carrier gas, helium (1 mL/minute) was used. A temperature of a sample vaporizing chamber, a temperature of an ion source, ionizing voltage and emission current were set to 300° C., 200° C., 70 eV and 150 uA, respectively. A sample was dissolved in acetone and prepared to be a 1 weight % solution, and then 1 microliter of the solution obtained was injected into the sample vaporizing chamber. As a recorder, GCMS Solution System made by Shimadzu Corporation was used.

Gas chromatographic analysis: For measurement, GC-2010 Gas Chromatograph made by Shimadzu Corporation was used. As a column, a capillary column DB-1 (length 60 m, bore 0.25 mm, film thickness 0.25 μm) made by Agilent Technologies, Inc. was used. As a carrier gas, helium (1 mL/minute) was used. A temperature of a sample vaporizing chamber and a temperature of a detector (FID) were set to 300° C. and 300° C., respectively. A sample was dissolved in acetone and prepared to be a 1 weight % solution, and then 1 microliter of the solution obtained was injected into the sample vaporizing chamber. As a recorder, GC Solution System made by Shimadzu Corporation or the like was used.

HPLC analysis: For measurement, Prominence (LC-20AD; SPD-20A) made by Shimadzu Corporation was used. As a column, YMC-Pack ODS-A (length 150 mm, bore 4.6 mm, particle diameter 5 μm) made by YMC Co., Ltd. was used. As an eluate, acetonitrile and water were appropriately mixed and used. As a detector, a UV detector, an RI detector, a CORONA detector or the like was appropriately used. When the UV detector was used, a detection wavelength was set to 254 nanometers. A sample was dissolved in acetonitrile and prepared to be a 0.1 weight % solution, and then 1 microliter of the solution was injected into a sample chamber. As a recorder, C-R7Aplus made by Shimadzu Corporation was used.

Ultraviolet-Visible spectrophotometry: For measurement, PharmaSpec UV-1700 made by Shimadzu Corporation was used. A detection wavelength was adjusted in the range of 190 nanometers to 700 nanometers. A sample was dissolved in acetonitrile and prepared to be a 0.01 mmol/L solution, and measurement was carried out by putting the solution in a quartz cell (optical path length: 1 cm).

Sample for measurement: Upon measuring phase structure and a transition temperature (a clearing point, a melting point, a polymerization starting temperature or the like), the compound itself was used as a sample. Upon measuring physical properties such as maximum temperature of a nematic phase, viscosity, optical anisotropy and dielectric anisotropy, a mixture of the compound and a base liquid crystal was used as a sample.

Extrapolation method: When the sample prepared by mixing the compound with the base liquid crystal was used, measurement was carried out as described below. The sample was prepared by mixing 15% by weight of the compound and 85% by weight of the base liquid crystal. From a measured value of the sample, an extrapolated value was calculated according to the following equation, and the calculated value was described: [extrapolated value]=(100×[measured value of a sample]−[% by weight of a base liquid crystal]×[measured value of the base liquid crystal])/[% by weight of a compound].

When crystals (or a smectic phase) precipitated at 25° C. at the ratio, a ratio of the compound to base liquid crystal was changed in the order of (10% by weight:90% by weight), (5% by weight:95% by weight) and (1% by weight:99% by weight), and physical properties of the sample were measured at a ratio at which no crystal (or no smectic phase) precipitated at 25° C. In addition, unless otherwise noted, the ratio of the compound to the base liquid crystal was (15% by weight:85% by weight).

When the dielectric anisotropy of the compound was zero or positive, base liquid crystal (A) described below was used. A proportion of each component was expressed in terms of weight percent (% by weight).

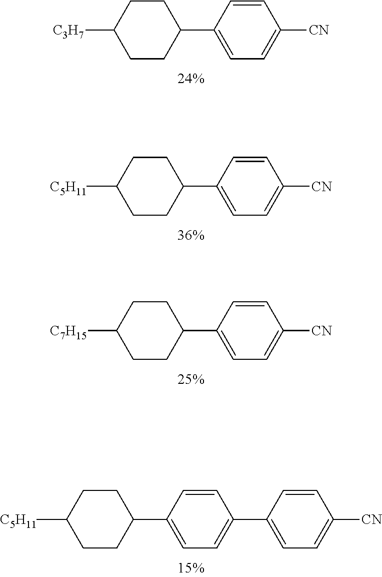

When the dielectric anisotropy of the compound was zero or negative, base liquid crystal (B) described below was used. A proportion of each component was expressed in terms of weight percent (% by weight).

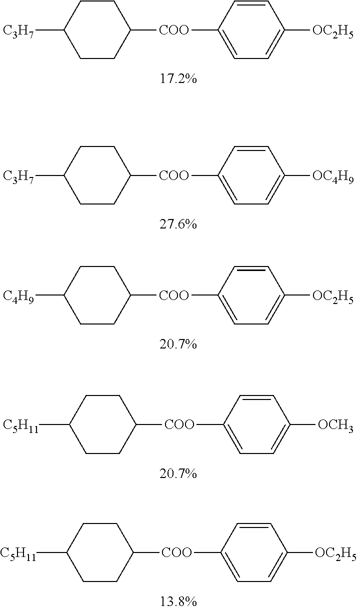

Measuring method: Physical properties were measured according to methods described below. Most of the methods are described in the Standard of Japan Electronics and Information Technology Industries Association (JEITA) discussed and established in JEITA (JEITA ED-2521B). A modified method was also applied. No thin film transistor (TFT) was attached to a TN device used for measurement.

(1) Phase structure: A sample was placed on a hot plate in a melting point apparatus (FP-52 Hot Stage made by Mettler-Toledo International Inc.) equipped with a polarizing microscope. A state of phase and a change thereof were observed with the polarizing microscope while the sample was heated at a rate of 3° C. per minute, and a kind of the phase was specified.

(2) Transition temperature (° C.): For measurement, a differential scanning calorimeter, Diamond DSC System, made by PerkinElmer, Inc., or a high sensitivity differential scanning calorimeter, X-DSC7000, made by SII NanoTechnology Inc. was used. A sample was heated and then cooled at a rate of 3° C. per minute, and a starting point of an endothermic peak or an exothermic peak caused by a phase change of the sample was determined by extrapolation, and thus a transition temperature was determined. A melting point and a polymerization starting temperature of a compound were also measured using the apparatus. Temperature at which a compound undergoes transition from a solid to a liquid crystal phase such as the smectic phase and the nematic phase may be occasionally abbreviated as “minimum temperature of the liquid crystal phase.” Temperature at which the compound undergoes transition from the liquid crystal phase to liquid may be occasionally abbreviated as “clearing point.”

A crystal was expressed as C. When the crystals were distinguishable into two kinds, each of the crystals was expressed as C₁ or C₂. The smectic phase or the nematic phase was expressed as S or N. When a phase such as smectic A phase, smectic B phase, smectic C phase and smectic F phase was distinguishable, the phase was expressed as S_{A}, S_{B}, S_{C} and S_{F}, respectively. A liquid (isotropic) was expressed as I. A transition temperature was expressed as “C 50.0 N 100.0 I,” for example. The expression indicates that a transition temperature from the crystals to the nematic phase is 50.0° C., and a transition temperature from the nematic phase to the liquid is 100.0° C.

(3) Compatibility of compound: Samples in which the base liquid crystal and the compound were mixed for proportions of the compounds to be 20% by weight, 15% by weight, 10% by weight, 5% by weight, 3% by weight or 1% by weight were prepared. The samples were put in glass vials, and kept in freezers at −10° C. or −20° C. for a predetermined period of time. Whether a nematic phase of the samples was maintained or crystals (or a smectic phase) precipitated was observed. Conditions on which the nematic phase was maintained were used as a measure of the compatibility. Proportions of the compounds and each temperature in the freezers may be occasionally changed when necessary.

(4) Maximum temperature of nematic phase (T_{NI} or NI; ° C.): A sample was placed on a hot plate in a melting point apparatus equipped with a polarizing microscope, and heated at a rate of 1° C. per minute. Temperature when part of the sample began to change from a nematic phase to an isotropic liquid was measured. When the sample was a mixture of compound (1) and the base liquid crystal, the maximum temperature was expressed in terms of a symbol T_{NI}. The value was calculated using the extrapolation method described above. When the sample was a mixture of compound (1) and a compound selected from compounds (2) to (15), the measured value was expressed in terms of a symbol NI. A maximum temperature of the nematic phase may be occasionally abbreviated as “maximum temperature.”

(5) Minimum temperature of nematic phase (T_{C}; ° C.): Samples each having a nematic phase were put in glass vials and kept in freezers at temperatures of 0° C., −10° C., −20° C., −30° C. and −40° C. for 10 days, and then liquid crystal phases were observed. For example, when the sample was maintained in the nematic phase at −20° C. and changed to crystals or a smectic phase at −30° C., T_{c} was expressed as T_{c}<−20° C. A minimum temperature of the nematic phase may be occasionally abbreviated as “minimum temperature.”

(6) Viscosity (bulk viscosity; η; measured at 20° C.; mPa·s): For measurement, a cone-plate (E type) rotational viscometer made by Tokyo Keiki Inc. was used.

(7) Optical anisotropy (refractive index anisotropy; measured at 25° C.; Δn): Measurement was carried out by an Abbe refractometer with a polarizing plate mounted on an ocular, using light at a wavelength of 589 nanometers. A surface of a main prism was rubbed in one direction, and then a sample was added dropwise onto the main prism. A refractive index (n∥) was measured when a direction of polarized light was parallel to a direction of rubbing. A refractive index (n⊥) was measured when the direction of polarized light was perpendicular to the direction of rubbing. A value of optical anisotropy (Δn) was calculated from an equation: Δn=n∥−n⊥.

(8) Specific resistance (ρ; measured at 25° C.; Ωcm): Into a vessel equipped with electrodes, 1.0 milliliter of sample was injected. A direct current voltage (10 V) was applied to the vessel, and a direct current after 10 seconds was measured. Specific resistance was calculated from the following equation: (specific resistance)={(voltage)× (electric capacity of a vessel)}/{(direct current)× (dielectric constant of vacuum)}.

(9) Voltage holding ratio (VHR-1; measured at 25° C.; %): A TN device used for measurement had a polyimide alignment film, and a distance (cell gap) between two glass substrates was 5 micrometers. A sample was put in the device, and then the device was sealed with an ultraviolet-curable adhesive. The device was charged by applying a pulse voltage (60 microseconds at 5 V). A decaying voltage was measured for 16.7 milliseconds with a high-speed voltmeter, and area A between a voltage curve and a horizontal axis in a unit cycle was determined. Area B is an area without decay. A voltage holding ratio is expressed in terms of a percentage of area A to area B.

(10) Voltage holding ratio (VHR-2; measured at 80° C.; %): A voltage holding ratio was measured according to the method described above except that the voltage holding ratio was measured at 80° C. in place of 25° C. The results obtained were expressed in terms of a symbol VHR-2.

(11) Flicker rate (measured at 25° C.; %): For measurement, 3298F Multimedia Display Tester made by Yokogawa Electric Corporation was used. A light source was LED. A sample was put in a normally black mode FFS device in which a distance (cell gap) between two glass substrates was 3.5 micrometers, and a rubbing direction was anti-parallel. The device was sealed with an ultraviolet-curable adhesive. Voltage was applied to the device, and a voltage having a maximum amount of light transmitted through the device was measured. A flicker rate displayed thereon was read by bringing a sensor unit close to the device while voltage was applied to the device.

The measuring method of the physical properties may be different between a sample having positive dielectric anisotropy and a sample having negative dielectric anisotropy. When the dielectric anisotropy was positive, the measuring method was described in section (12a) to section (16a). When the dielectric anisotropy was negative, the measuring method was described in section (12b) to section (16b).

(12a) Viscosity (rotational viscosity; γ1; measured at 25° C.; mPa·s; for a sample having positive dielectric anisotropy): Measurement was carried out according to a method described in M. Imai et al., Molecular Crystals and Liquid Crystals, Vol. 259, p. 37 (1995). A sample was put in a TN device in which a twist angle was 0 degrees, and a distance (cell gap) between two glass substrates was 5 micrometers. Voltage was applied stepwise to the device from 16 V to 19.5 V at an increment of 0.5 V. After a period of 0.2 second with no voltage application, voltage was repeatedly applied under conditions of only one rectangular wave (rectangular pulse; 0.2 second) and no voltage application (2 seconds). A peak current and a peak time of transient current generated by the applied voltage were measured. A value of rotational viscosity was obtained from the measured values and equation (8) on page 40 of the paper presented by M. Imai et al. A value of dielectric anisotropy required for the calculation was determined using the device by which the rotational viscosity was measured and by a method described below.

(12b) Viscosity (rotational viscosity; γ1; measured at 25° C.; mPa·s; for a sample having negative dielectric anisotropy): Measurement was carried out according to the method described in M. Imai et al., Molecular Crystals and Liquid Crystals, Vol. 259, p. 37 (1995). A sample was put in a VA device in which a distance (cell gap) between two glass substrates was 20 micrometers. Voltage was applied stepwise to the device from 39 V to 50 V at an increment of 1 V. After a period of 0.2 second with no voltage application, voltage was repeatedly applied under conditions of only one rectangular wave (rectangular pulse; 0.2 second) and no voltage application (2 seconds). A peak current and a peak time of transient current generated by the applied voltage were measured. A value of rotational viscosity was obtained from the measured values and equation (8) on page 40 of the paper presented by M. Imai et al. Dielectric anisotropy required for the calculation was measured in a section of dielectric anisotropy described below.

(13a) Dielectric anisotropy (As; measured at 25° C.; for a sample having positive dielectric anisotropy): A sample was put in a TN device in which a distance (cell gap) between two glass substrates was 9 micrometers and a twist angle was 80 degrees. Sine waves (10 V, 1 kHz) were applied to the device, and after 2 seconds, a dielectric constant (ε∥) of liquid crystal molecules in a major axis direction was measured. Sine waves (0.5 V, 1 kHz) were applied to the device, and after 2 seconds, a dielectric constant (ε⊥) of liquid crystal molecules in a minor axis direction was measured. A value of dielectric anisotropy was calculated from an equation: Δε=ε∥−ε⊥.

(13b) Dielectric anisotropy (Δε; measured at 25° C.; for a sample having negative dielectric anisotropy): A value of dielectric anisotropy was calculated from an equation: Δε=ε∥−ε⊥. A dielectric constant (ε∥ and ε⊥) was measured as described below.

1) Measurement of dielectric constant (ε∥): An ethanol (20 mL) solution of octadecyltriethoxysilane (0.16 mL) was applied to a well-cleaned glass substrate. After rotating the glass substrate with a spinner, the glass substrate was heated at 150° C. for 1 hour. A sample was put in a VA device in which a distance (cell gap) between two glass substrates was 4 micrometers, and the device was sealed with an ultraviolet-curable adhesive. Sine waves (0.5 V, 1 kHz) were applied to the device, and after 2 seconds, a dielectric constant (ε∥) of liquid crystal molecules in a major axis direction was measured.

2) Measurement of dielectric constant (ε⊥): A polyimide solution was applied to a well-cleaned glass substrate. After calcining the glass substrate, rubbing treatment was applied to the alignment film obtained. A sample was put in a TN device in which a distance (cell gap) between two glass substrates was 9 micrometers and a twist angle was 80 degrees. Sine waves (0.5 V, 1 kHz) were applied to the device, and after 2 seconds, a dielectric constant (ε⊥) of liquid crystal molecules in a minor axis direction was measured.

(14a) Elastic constant (K; measured at 25° C.; pN; for a sample having positive dielectric anisotropy): For measurement, HP4284A LCR Meter made by Yokogawa-Hewlett-Packard Co. was used. A sample was put in a horizontal alignment device in which a distance (cell gap) between two glass substrates was 20 micrometers. An electric charge from 0 V to 20 V was applied to the device, and electrostatic capacity (C) and applied voltage (V) were measured. The measured values were fitted to equation (2.98) and equation (2.101) on page 75 of “Liquid Crystal Device Handbook (Ekisho Debaisu Handobukku in Japanese; Nikkan Kogyo Shimbun, Ltd.),” and values of K₁₁ and K₃₃ were obtained from equation (2.99). Next, K₂₂ was calculated using the previously determined values of K₁₁ and K₃₃ in equation (3.18) on page 171. Elastic constant K was expressed in terms of a mean value of the thus determined K₁₁, K₂₂ and K₃₃.

(14b) Elastic constant (K₁₁ and K₃₃; measured at 25° C.; pN; for a sample having negative dielectric anisotropy): For measurement, Elastic Constant Measurement System Model EC-1 made by TOYO Corporation was used. A sample was put in a vertical alignment device in which a distance (cell gap) between two glass substrates was 20 micrometers. An electric charge from 20 V to 0 V was applied to the device, and electrostatic capacity (C) and applied voltage (V) were measured. The measured values were fitted to equation (2.98) and equation (2.101) on page 75 of “Liquid Crystal Device Handbook (Ekisho Debaisu Handobukku in Japanese; Nikkan Kogyo Shimbun, Ltd.),” and values of elastic constants were obtained from equation (2.100).

(15a) Threshold voltage (Vth; measured at 25° C.; V; for a sample having positive dielectric anisotropy): For measurement, an LCD-5100 luminance meter made by Otsuka Electronics Co., Ltd. was used. A light source was a halogen lamp. A sample was put in a normally white mode TN device in which a distance (cell gap) between two glass substrates was 0.45/Δn (μm) and a twist angle was 80 degrees. A voltage (32 Hz, rectangular waves) to be applied to the device was stepwise increased from 0 V to 10 V at an increment of 0.02 V. On the occasion, the device was irradiated with light from a direction perpendicular to the device, and an amount of light transmitted through the device was measured. A voltage-transmittance curve was prepared, in which the maximum amount of light corresponds to 100% transmittance and the minimum amount of light corresponds to 0% transmittance. A threshold voltage is expressed in terms of voltage at 90% transmittance.

(15b) Threshold voltage (Vth; measured at 25° C.; V; for a sample having negative dielectric anisotropy): For measurement, an LCD-5100 luminance meter made by Otsuka Electronics Co., Ltd. was used. A light source was a halogen lamp. A sample was put in a normally black mode VA device in which a distance (cell gap) between two glass substrates was 4 micrometers and a rubbing direction was anti-parallel, and the device was sealed with an ultraviolet-curable adhesive. A voltage (60 Hz, rectangular waves) to be applied to the device was stepwise increased from 0 V to 20 V at an increment of 0.02 V. On the occasion, the device was irradiated with light from a direction perpendicular to the device, and an amount of light transmitted through the device was measured. A voltage-transmittance curve was prepared, in which the maximum amount of light corresponds to 100% transmittance and the minimum amount of light corresponds to 0% transmittance. A threshold voltage is expressed in terms of voltage at 10% transmittance.

(16a) Response time (i; measured at 25° C.; ms; for a sample having positive dielectric anisotropy): For measurement, an LCD-5100 luminance meter made by Otsuka Electronics Co., Ltd. was used. A light source was a halogen lamp. A low-pass filter was set to 5 kHz. A sample was put in a normally white mode TN device in which a distance (cell gap) between two glass substrates was 5.0 micrometers and a twist angle was 80 degrees. A voltage (rectangular waves; 60 Hz, 5 V, 0.5 second) was applied to the device. On the occasion, the device was irradiated with light from a direction perpendicular to the device, and an amount of light transmitted through the device was measured. The maximum amount of light corresponds to 100% transmittance, and the minimum amount of light corresponds to 0% transmittance. A rise time (τr; millisecond) was expressed in terms of time required for a change from 90% transmittance to 10% transmittance. A fall time (τf; millisecond) was expressed in terms of time required for a change from 10% transmittance to 90% transmittance. A response time was expressed by a sum of the rise time and the fall time thus determined.

(16b) Response time (τ; measured at 25° C.; ms; for a sample having negative dielectric anisotropy): For measurement, an LCD-5100 luminance meter made by Otsuka Electronics Co., Ltd. was used. A light source was a halogen lamp. A low-pass filter was set to 5 kHz. A sample was put in a normally black mode PVA device in which a distance (cell gap) between two glass substrates was 3.2 micrometers and a rubbing direction was anti-parallel. The device was sealed with an ultraviolet-curable adhesive. The device was applied with a voltage of a little exceeding a threshold voltage for 1 minute, and then was irradiated with ultraviolet light of 23.5 mW/cm² for 8 minutes, while applying a voltage of 5.6 V. A voltage (rectangular waves; 60 Hz, 10 V, 0.5 second) was applied to the device. On the occasion, the device was irradiated with light from a direction perpendicular to the device, and an amount of light transmitted through the device was measured. The maximum amount of light corresponds to 100% transmittance, and the minimum amount of light corresponds to 0% transmittance. A response time was expressed in terms of time required for a change from 90% transmittance to 10% transmittance (fall time; millisecond).

(17) Line image sticking parameter (LISP; %): Line image sticking parameter was generated by giving electric stress to a liquid crystal display device. Luminance in a region having the line image sticking parameter and luminance in the remaining region were measured. A proportion at which the luminance was decreased by the line image sticking parameter was calculated, and magnitude of the line image sticking parameter was expressed in terms of the proportion.

17a) Measurement of luminance: An image of the device was taken using Imaging Colorimeters and Photometers (made by Radiant Zemax, LLC, PM-1433F-0). Luminance in each region of the device was calculated by analyzing the image by using software (Prometric 9.1, made by Radiant Imaging, Inc.).

17b) Setting of stress voltage: A sample was put in an FFS device (16 cells, vertically 4 cells×horizontally 4 cells) having a cell gap of 3.5 micrometers and having a matrix structure, and the device was sealed with an ultraviolet-curable adhesive. Polarizing plates were arranged on an upper surface and a lower surface of the device, respectively, to be perpendicular to each other in polarization axes. The device was irradiated with light, and a voltage (rectangular waves, 60 Hz) was applied thereto. The voltage was stepwise increased in the range of 0 V to 7.5 V at an increment of 0.1 V, and luminance of transmitted light at each voltage was measured. Voltage when luminance became maximum was abbreviated as V255. Voltage when luminance reached 21.6% of V225 (more specifically, 127 gradations) was abbreviated as V127.

17c) Conditions of stress: V255 (rectangular waves, 30 Hz) and 0.5V (rectangular waves, 30 Hz) were applied to the device under conditions of 60° C. and 23 hours, and a checker pattern was displayed on the device. Next, V127 (rectangular waved, 0.25 Hz) was applied thereto, and luminance was measured under conditions of exposure time of 4,000 milliseconds.

17d) Calculation of line image sticking parameter: Among 16 cells, 4 cells (vertically 2 cells×horizontally 2 cells) in a center part were used for calculation. The 4 cells were divided into 25 regions (vertically 5 cells×horizontally 5 cells). Average luminance in 4 regions (vertically 2 cells×horizontally 2 cells) in four corners was abbreviated as luminance A. A region where the 4 regions in the four corners were excluded from the 25 regions had a cross shape. In 4 regions excluding a center crossing area from the region having the cross shape, a minimum value of luminance was abbreviated as luminance B. The line image sticking parameter was calculated from the following equation: (line image sticking parameter)=(luminance A−luminance B)/luminance A×100.

### Synthesis Example 1

### Synthesis of Compound (277)

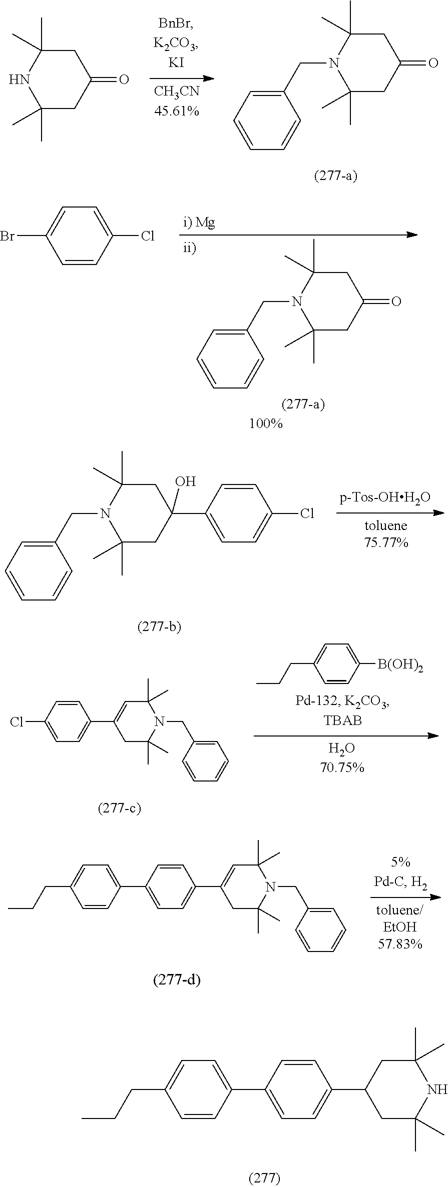

### First Step:

In a 300 mL three-necked flask, 15.0 g (96.62 mmol) of 2,2,6,6-tetramethylpiperidine-4-one, 19.832 g (115.95 mmol) of benzyl bromide, 26.708 g (193.25 mmol) of potassium carbonate, 1.492 g (8.99 mmol) of potassium iodide and acetonitrile (120 mL) were put, and the resultant mixture was heated and stirred at 80 to 85° C. After 24 hours, the reaction mixture was returned to room temperature, and poured into water, and the resultant mixture was subjected to extraction with toluene (100 mL×3). The extracted solution was washed with water until pH reached 7, and dried over anhydrous magnesium sulfate, and then concentrated under reduced pressure. Petroleum ether (24 mL) was added to a residue, and the resultant mixture was recrystallized at −25° C. for 2 hours, and then ethanol (24 mL) was added thereto, and the resultant mixture was recrystallized at −25° C. for 1 hour to obtain 10.814 g (44.07 mmol, Y: 45.61%) (purity: 99.60%) of a benzyl protecting body (277-a).

### Second Step:

To a suspension prepared by putting 1.672 g (72.72 mmol) of magnesium and tetrahydrofuran (20 mL) in a 200 mL three-necked flask, and 12.660 g (66.11 mmol) of 1-bromo-4-chlorobenzene was added dropwise at 45 to 55° C. After 1 hour, a solution obtained by dissolving 10.814 g (44.07 mmol) of the benzyl protecting body (277-a) in THF (50 mL) was added dropwise thereto. After 2 hours, the reaction mixture was ice-cooled, and then dilute hydrochloric acid was added dropwise thereto until pH reached 4 to 5. Subsequently, a saturated aqueous solution of sodium hydrogencarbonate was added dropwise thereto until pH reached 8 to 9. The solution obtained was subjected to extraction with toluene (50 mL×3), and then the resulting extract was washed with water until pH reached 7. The resultant solution was dried over anhydrous magnesium sulfate, and then concentrated under reduced pressure to obtain 15.774 g (44.07 mmol, Y: 100%) (purity: 90.80%) of a chloro body (277-b).

### Third Step:

Then, 15.774 g (44.07 mmol) of the chloro body (277-b) was dissolved in toluene (20 mL), and then 1.577 g (0.1 wt %) of p-toluenesulfonic acid monohydrate was added thereto, and the resultant mixture was heated and stirred at 105 to 110° C. After 2 hours, the reaction mixture was returned to room temperature, and then a saturated aqueous solution of sodium hydrogencarbonate was added dropwise thereto until pH reached 8 to 9. Subsequently, the resultant solution was washed with water until pH reached 7, and dried over anhydrous magnesium sulfate, and then concentrated under reduced pressure. A residue was dissolved in heptane (15 mL), and the resultant solution was recrystallized at −25° C. for 2 hours to obtain 11.350 g (33.39 mmol, Y: 75.77%) (purity: 90.80%) of a leaving body (277-c).

### Fourth Step:

To a 200 mL three-necked flask, 11.350 g (33.39 mmol) of the leaving body (277-c), 6.572 g (40.07 mmol) of 4-propyl phenylboronic acid, 0.0236 g (0.033 mmol) of pd-132, 6.686 g (66.78 mmol) of potassium carbonate, 3.229 g (10.02 mmol) of tetrabutylammonium bromide and water (100 mL) were added, and the resultant mixture was heated under reflux. After 4 hours, the reaction mixture was returned to room temperature, and poured into water, and the resultant mixture was subjected to extraction with toluene (50 mL×3). The extracted solution was washed with water until pH reached 7, and dried over anhydrous magnesium sulfate and then concentrated under reduced pressure. A residue was dissolved in a mixed solvent of toluene (5 mL)-ethanol (20 mL), and the resultant mixture was recrystallized at −25° C. for 12 hours to obtain 10.01 g (23.62 mmol, Y: 70.75%) (purity: 99.09%) of a condensation product (277-d).

### Fifth Step:

Then, 10.01 g (23.62 mmol) of the condensation product (277-d) was dissolved in a mixed solvent of toluene (80 mL)-ethanol (20 mL), and then 0.5 g (0.05 wt %) of Pd—C was added thereto, and the resultant mixture was stirred at 55 to 60° C. under a hydrogen atmosphere. After 32 hours, Pd—C was filtrated off and the resulting filtrate was concentrated under reduced pressure. A residue was passed through a silica gel column and dissolved in heptane (20 mL), and the resultant solution was recrystallized at −25° C. for 2 hours to obtain 4.58 g (13.66 mmol, Y: 57.83%) (purity: 99.94%) of an objective compound (277).

¹H-NMR (ppm; CDCl₃): δ 7.53 (d, J=8.2 Hz, 2H), 7.50 (d, J=8.6 Hz, 2H), 7.29 (d, J=8.2 Hz, 2H), 7.24 (d, J=8.6 Hz, 2H), 3.07 (tt, J=12.8 Hz, J=3.2 Hz, 1H), 2.62 (t, J=7.8 Hz, 2H), 1.81 (dd, J=13.1 Hz, J=3.0 Hz, 2H), 1.72-1.64 (m, 2H), 1.58 (brs, 1H), 1.34-1.29 (m, 8H), 1.17 (s, 6H), 0.98 (t, J=7.2 Hz, 3H).

### Synthesis Example 2

### Synthesis of Compound (265)

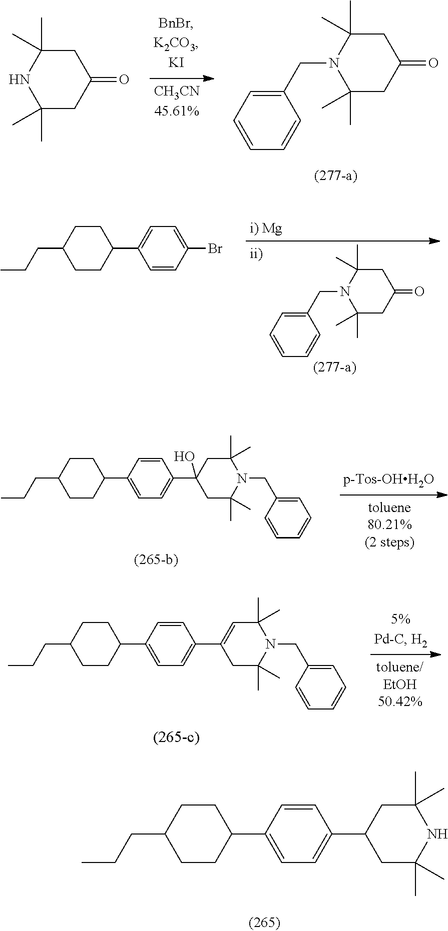

### First Step:

In a 300 mL three-necked flask, 15.0 g (96.62 mmol) of 2,2,6,6-tetramethylpiperidine-4-one, 19.832 g (115.95 mmol) of benzyl bromide, 26.708 g (193.25 mmol) of potassium carbonate, 1.492 g (8.99 mmol) of potassium iodide and acetonitrile (120 mL) were put, and the resultant mixture was heated and stirred at 80 to 85° C. After 24 hours, the reaction mixture was returned to room temperature, and poured into water, and the resultant mixture was subjected to extraction with toluene (100 mL×3). The extracted solution was washed with water until pH reached 7, and dried over anhydrous magnesium sulfate, and then concentrated under reduced pressure. Petroleum ether (24 mL) was added to a residue and the resultant mixture was recrystallized at −25° C. for 2 hours, and then ethanol (24 mL) was added thereto and the resultant mixture was recrystallized at −25° C. for 1 hour, to obtain 10.814 g (44.07 mmol, Y: 45.61%) (purity: 99.60%) of a benzyl protector (277-a).

### Second Step:

In a 200 mL three-necked flask, 1.672 g (72.72 mmol) of magnesium and tetrahydrofuran (20 mL) was put to prepare a suspension, and 18.590 g (66.11 mmol) of 1-bromo-4-(4-propylcyclohexyl)benzene was added dropwise onto the suspension at 55 to 65° C. After 1 hour, a solution obtained by dissolving 10.814 g (44.07 mmol) of the benzyl protector (277-a) in THF (30 mL) was added dropwise thereto. After 1 hour, the reaction mixture was ice-cooled, and then dilute hydrochloric acid was added dropwise thereto until pH reached 4 to 5. Subsequently, a saturated aqueous solution of sodium hydrogencarbonate was added dropwise thereto until pH reached 8 to 9. The solution obtained was subjected to extraction with toluene (50 mL×3), and then the resulting extract was washed with water until pH reached 7, and dried over anhydrous magnesium sulfate, and then concentrated under reduced pressure to obtain 29.598 g (44.07 mmol, Y: 100%) of an alcohol body (265-b).

### Third Step:

Then, 29.598 g (44.07 mmol) of the alcohol body (265-b) was dissolved in toluene (150 mL), and then 2.960 g (0.1 wt %) of p-toluenesulfonic acid monohydrate was added thereto, and the resultant mixture was heated and stirred at 75 to 110° C. After 2 hours, the reaction mixture was returned to room temperature, and then a saturated aqueous solution of sodium hydrogencarbonate was added dropwise thereto until pH reached 8 to 9. Subsequently, the resultant solution was washed with water until pH reached 7, and dried over anhydrous magnesium sulfate, and then concentrated under reduced pressure. A residue was dissolved in toluene (15 mL)-heptane (30 mL), and the resultant solution was recrystallized at −25° C. for 2 hours to obtain 15.189 g (35.35 mmol, Y: 80.21%) (purity: 99.51%) of a leaving body (265-c).

### Fifth Step:

Then, 15.189 g (35.35 mmol) of the condensation product (265-c) was dissolved in a mixed solvent of toluene (90 mL)-ethanol (30 mL), and 0.75 g (0.05 wt %) of Pd—C was added thereto, and the resultant mixture was stirred at 55 to 60° C. under a hydrogen atmosphere. After 24 hours, Pd—C was filtrated off and the resulting filtrate was concentrated under reduced pressure. A residue was passed through a silica gel column and dissolved in ethanol (20 mL)-heptane (40 mL), and the resultant solution was recrystallized at −25° C. for 1 hour to obtain 6.09 g (17.82 mmol, Y: 50.42) (purity: 99.91%) of an objective compound (265).

¹H-NMR (ppm; CDCl₃): δ 7.17-7.13 (m, 4H), 2.99 (tt, J=10.1 Hz, J=3.1 Hz, 1H), 2.44 (tt, J=12.1 Hz, J=3.1 Hz, 1H), 1.90-1.83 (m, 4H), 1.77 (dd, J=13.3 Hz, J=3.2 Hz, 1H), 1.59 (brs, 1H), 1.48-1.18 (m, 15H), 1.14 (s, 6H), 0.90 (t, J=7.2 Hz, 3H).

Compound (No. 1) to compound (No. 312) described below are prepared according to a synthetic method described in Synthesis example 1.

| No. | |
|---|---|
| (1) | 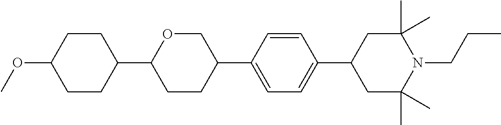 [SMILES: CCCN1C(C)(C)CC(c2ccc(C3CCC(C4CCC(OC)CC4)OC3)cc2)CC1(C)C] |
| (2) | 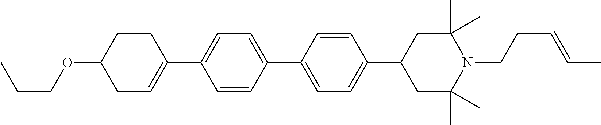 [SMILES: C/C=C/CCN1C(C)(C)CC(c2ccc(-c3ccc(C4=CCC(OCCC)CC4)cc3)cc2)CC1(C)C] |
| (3) | 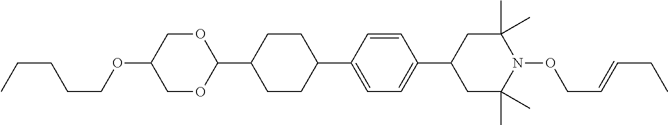 [SMILES: CC/C=C/CON1C(C)(C)CC(c2ccc(C3CCC(C4OCC(OCCCCC)CO4)CC3)cc2)CC1(C)C] |
| (4) | 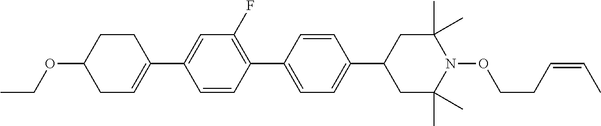 [SMILES: C/C=C\CCON1C(C)(C)CC(c2ccc(-c3ccc(C4=CCC(OCC)CC4)cc3F)cc2)CC1(C)C] |
| (5) | 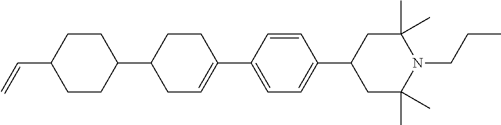 [SMILES: C=CC1CCC(C2CC=C(c3ccc(C4CC(C)(C)N(CCC)C(C)(C)C4)cc3)CC2)CC1] |
| (6) | 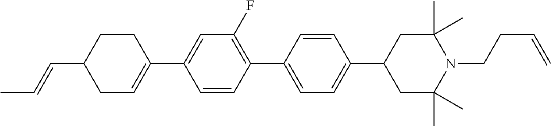 [SMILES: C=CCCN1C(C)(C)CC(c2ccc(-c3ccc(C4=CCC(/C=C/C)CC4)cc3F)cc2)CC1(C)C] |
| (7) | 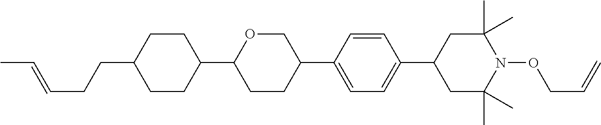 [SMILES: C=CCON1C(C)(C)CC(c2ccc(C3CCC(C4CCC(CC/C=C/C)CC4)OC3)cc2)CC1(C)C] |
| (8) | 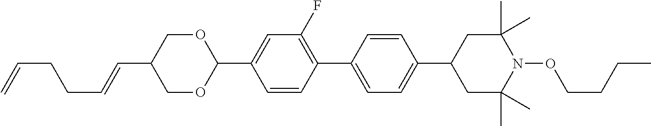 [SMILES: C=CCC/C=C/C1COC(c2ccc(-c3ccc(C4CC(C)(C)N(OCCCC)C(C)(C)C4)cc3)c(F)c2)OC1] |
| (9) | 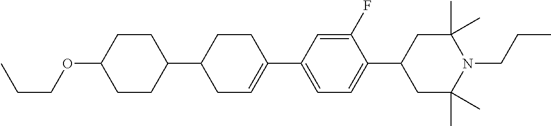 [SMILES: CCCOC1CCC(C2CC=C(c3ccc(C4CC(C)(C)N(CCC)C(C)(C)C4)c(F)c3)CC2)CC1] |
| (10) | 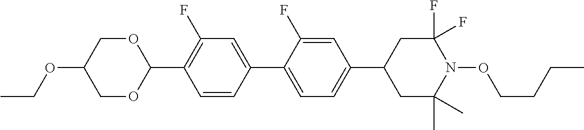 [SMILES: CCCCON1C(C)(C)CC(c2ccc(-c3ccc(C4OCC(OCC)CO4)c(F)c3)c(F)c2)CC1(F)F] |
| (11) | 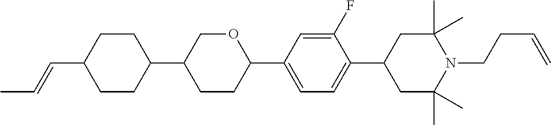 [SMILES: C=CCCN1C(C)(C)CC(c2ccc(C3CCC(C4CCC(/C=C/C)CC4)CO3)cc2F)CC1(C)C] |
| (12) | 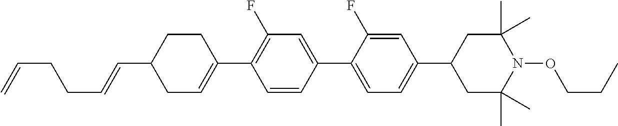 [SMILES: C=CCC/C=C/C1CC=C(c2ccc(-c3ccc(C4CC(C)(C)N(OCCC)C(C)(C)C4)cc3F)cc2F)CC1] |
| (13) | 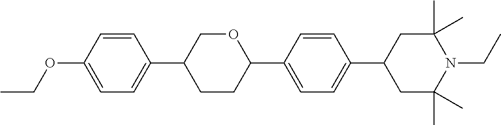 [SMILES: CCOc1ccc(C2CCC(c3ccc(C4CC(C)(C)N(CC)C(C)(C)C4)cc3)OC2)cc1] |
| (14) | 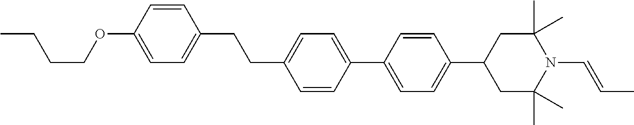 [SMILES: C/C=C/N1C(C)(C)CC(c2ccc(-c3ccc(CCc4ccc(OCCCC)cc4)cc3)cc2)CC1(C)C] |
| (15) | 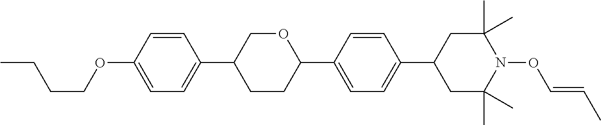 [SMILES: C/C=C/ON1C(C)(C)CC(c2ccc(C3CCC(c4ccc(OCCCC)cc4)CO3)cc2)CC1(C)C] |
| (16) | 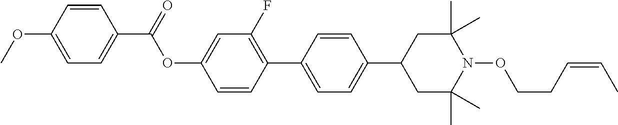 [SMILES: C/C=C\CCON1C(C)(C)CC(c2ccc(-c3ccc(OC(=O)c4ccc(OC)cc4)cc3F)cc2)CC1(C)C] |
| (17) | 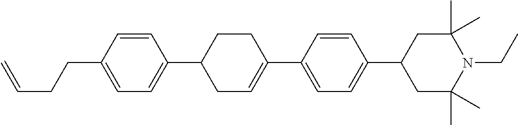 [SMILES: C=CCCc1ccc(C2CC=C(c3ccc(C4CC(C)(C)N(CC)C(C)(C)C4)cc3)CC2)cc1] |
| (18) | 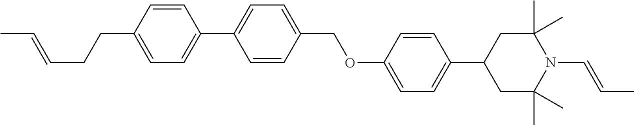 [SMILES: C/C=C/CCc1ccc(-c2ccc(COc3ccc(C4CC(C)(C)N(/C=C/C)C(C)(C)C4)cc3)cc2)cc1] |
| (19) | 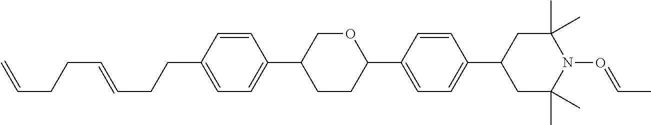 |
| (20) | 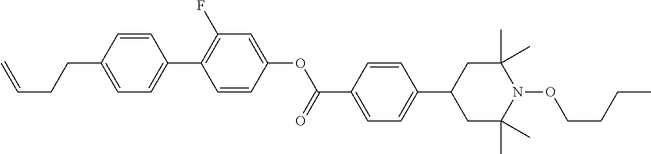 [SMILES: C=CCCc1ccc(-c2ccc(OC(=O)c3ccc(C4CC(C)(C)N(OCCCC)C(C)(C)C4)cc3)cc2F)cc1] |
| (21) | 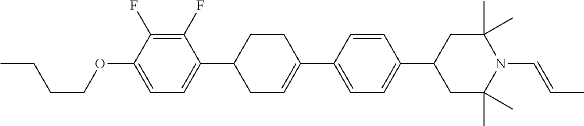 [SMILES: C/C=C/N1C(C)(C)CC(c2ccc(C3=CCC(c4ccc(OCCCC)c(F)c4F)CC3)cc2)CC1(C)C] |
| (22) | 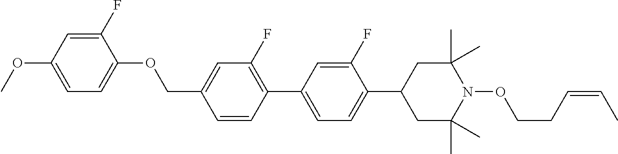 [SMILES: C/C=C\CCON1C(C)(C)CC(c2ccc(-c3ccc(COc4ccc(OC)cc4F)cc3F)cc2F)CC1(C)C] |
| (23) | 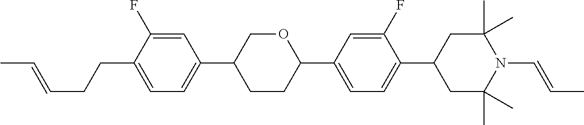 [SMILES: C/C=C/CCc1ccc(C2CCC(c3ccc(C4CC(C)(C)N(/C=C/C)C(C)(C)C4)c(F)c3)OC2)cc1F] |
| (24) | 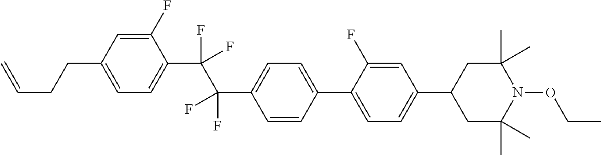 [SMILES: C=CCCc1ccc(C(F)(F)C(F)(F)c2ccc(-c3ccc(C4CC(C)(C)N(OCC)C(C)(C)C4)cc3F)cc2)c(F)c1] |
| (25) | 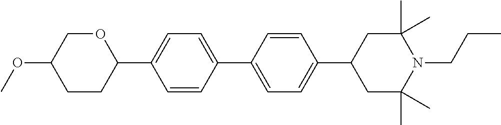 [SMILES: CCCN1C(C)(C)CC(c2ccc(-c3ccc(C4CCC(OC)CO4)cc3)cc2)CC1(C)C] |
| (26) | 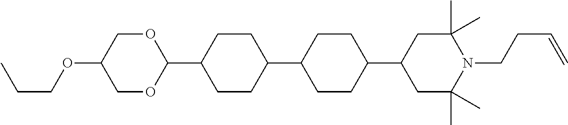 [SMILES: C=CCCN1C(C)(C)CC(C2CCC(C3CCC(C4OCC(OCCC)CO4)CC3)CC2)CC1(C)C] |
| (27) | 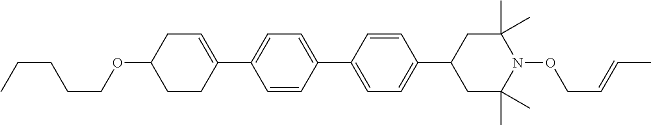 [SMILES: C/C=C/CON1C(C)(C)CC(c2ccc(-c3ccc(C4=CCC(OCCCCC)CC4)cc3)cc2)CC1(C)C] |
| (28) | 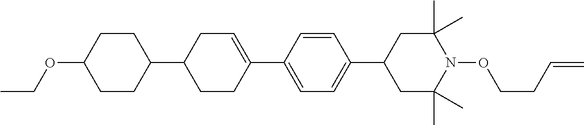 [SMILES: C=CCCON1C(C)(C)CC(c2ccc(C3=CCC(C4CCC(OCC)CC4)CC3)cc2)CC1(C)C] |
| (29) | 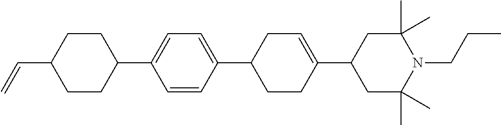 [SMILES: C=CC1CCC(c2ccc(C3CC=C(C4CC(C)(C)N(CCC)C(C)(C)C4)CC3)cc2)CC1] |
| (30) | 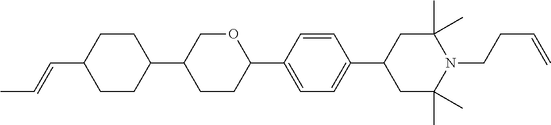 [SMILES: C=CCCN1C(C)(C)CC(c2ccc(C3CCC(C4CCC(/C=C/C)CC4)CO3)cc2)CC1(C)C] |
| (31) | 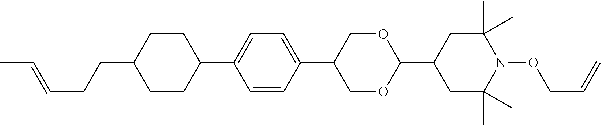 [SMILES: C=CCON1C(C)(C)CC(C2OCC(c3ccc(C4CCC(CC/C=C/C)CC4)cc3)CO2)CC1(C)C] |
| (32) | 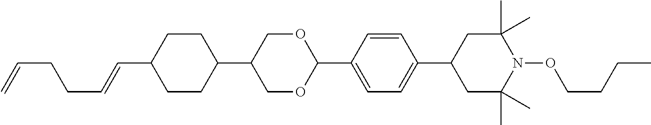 [SMILES: C=CCC/C=C/C1CCC(C2COC(c3ccc(C4CC(C)(C)N(OCCCC)C(C)(C)C4)cc3)OC2)CC1] |
| (33) | 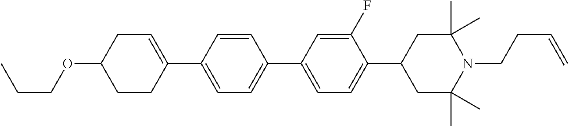 [SMILES: C=CCCN1C(C)(C)CC(c2ccc(-c3ccc(C4=CCC(OCCC)CC4)cc3)cc2F)CC1(C)C] |
| (34) | 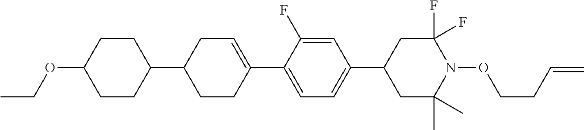 [SMILES: C=CCCON1C(C)(C)CC(c2ccc(C3=CCC(C4CCC(OCC)CC4)CC3)c(F)c2)CC1(F)F] |
| (35) | 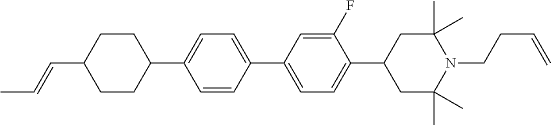 [SMILES: C=CCCN1C(C)(C)CC(c2ccc(-c3ccc(C4CCC(/C=C/C)CC4)cc3)cc2F)CC1(C)C] |
| (36) | 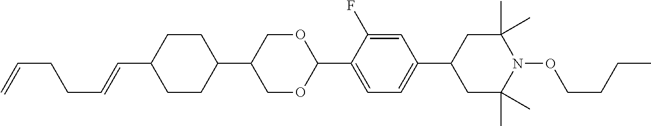 [SMILES: C=CCC/C=C/C1CCC(C2COC(c3ccc(C4CC(C)(C)N(OCCCC)C(C)(C)C4)cc3F)OC2)CC1] |
| (37) | 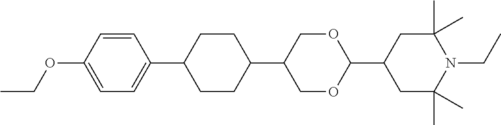 [SMILES: CCOc1ccc(C2CCC(C3COC(C4CC(C)(C)N(CC)C(C)(C)C4)OC3)CC2)cc1] |
| (38) | 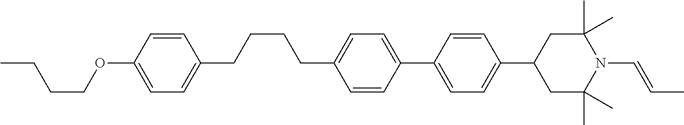 [SMILES: C/C=C/N1C(C)(C)CC(c2ccc(-c3ccc(CCCCc4ccc(OCCCC)cc4)cc3)cc2)CC1(C)C] |
| (39) | 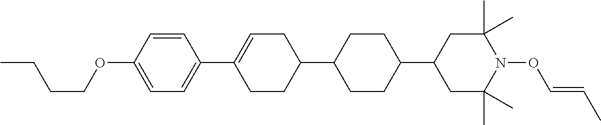 [SMILES: C/C=C/ON1C(C)(C)CC(C2CCC(C3CC=C(c4ccc(OCCCC)cc4)CC3)CC2)CC1(C)C] |
| (40) | 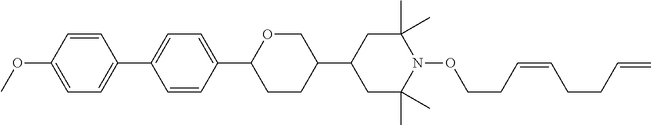 [SMILES: C=CCC/C=C\CCON1C(C)(C)CC(C2CCC(c3ccc(-c4ccc(OC)cc4)cc3)OC2)CC1(C)C] |
| (41) | 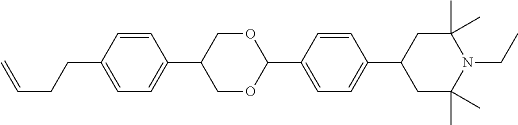 [SMILES: C=CCCc1ccc(C2COC(c3ccc(C4CC(C)(C)N(CC)C(C)(C)C4)cc3)OC2)cc1] |
| (42) | 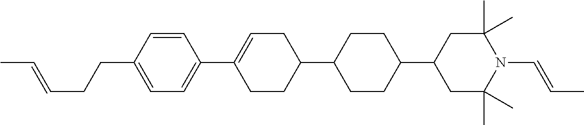 [SMILES: C/C=C/CCc1ccc(C2=CCC(C3CCC(C4CC(C)(C)N(/C=C/C)C(C)(C)C4)CC3)CC2)cc1] |
| (43) | 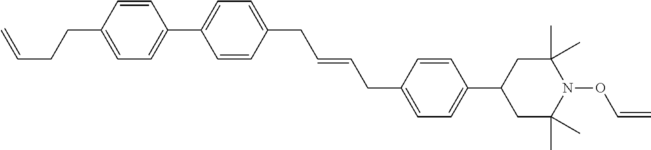 [SMILES: C=CCCc1ccc(-c2ccc(C/C=C/Cc3ccc(C4CC(C)(C)N(OC=C)C(C)(C)C4)cc3)cc2)cc1] |
| (44) | 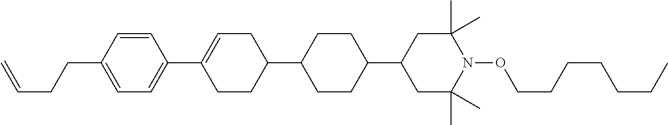 [SMILES: C=CCCc1ccc(C2=CCC(C3CCC(C4CC(C)(C)N(OCCCCCCC)C(C)(C)C4)CC3)CC2)cc1] |
| (45) | 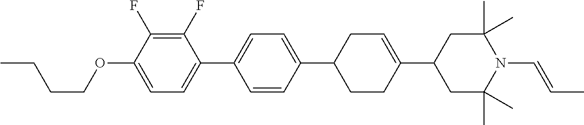 [SMILES: C/C=C/N1C(C)(C)CC(C2=CCC(c3ccc(-c4ccc(OCCCC)c(F)c4F)cc3)CC2)CC1(C)C] |
| (46) | 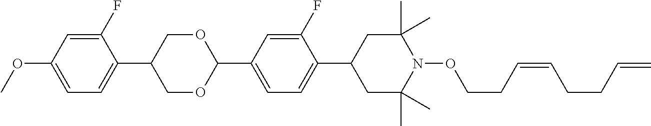 [SMILES: C=CCC/C=C\CCON1C(C)(C)CC(c2ccc(C3OCC(c4ccc(OC)cc4F)CO3)cc2F)CC1(C)C] |
| (47) | 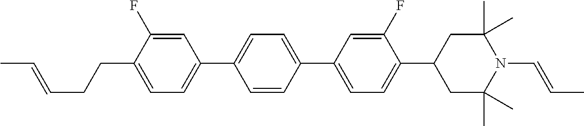 [SMILES: C/C=C/CCc1ccc(-c2ccc(-c3ccc(C4CC(C)(C)N(/C=C/C)C(C)(C)C4)c(F)c3)cc2)cc1F] |
| (48) | 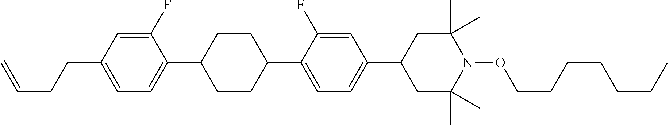 [SMILES: C=CCCc1ccc(C2CCC(c3ccc(C4CC(C)(C)N(OCCCCCCC)C(C)(C)C4)cc3F)CC2)c(F)c1] |
| (49) | 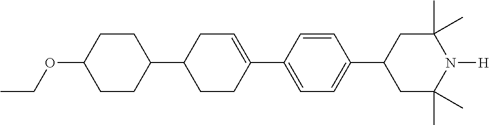 [SMILES: CCOC1CCC(C2CC=C(c3ccc(C4CC(C)(C)NC(C)(C)C4)cc3)CC2)CC1] |
| (50) | 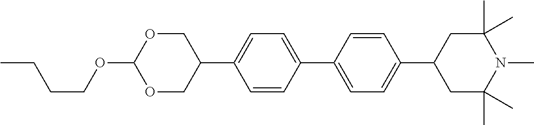 [SMILES: CCCCOC1OCC(c2ccc(-c3ccc(C4CC(C)(C)N(C)C(C)(C)C4)cc3)cc2)CO1] |
| (51) | 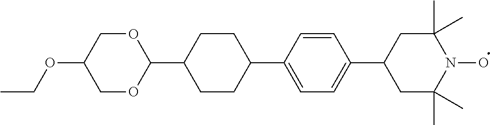 |
| (52) | 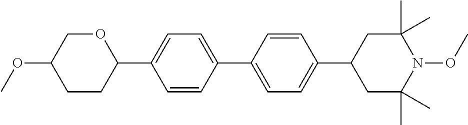 |
| (53) | 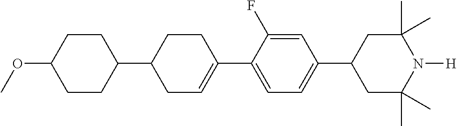 [SMILES: COC1CCC(C2CC=C(c3ccc(C4CC(C)(C)NC(C)(C)C4)cc3F)CC2)CC1] |
| (54) | 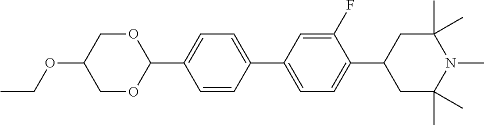 [SMILES: CCOC1COC(c2ccc(-c3ccc(C4CC(C)(C)N(C)C(C)(C)C4)c(F)c3)cc2)OC1] |
| (55) | 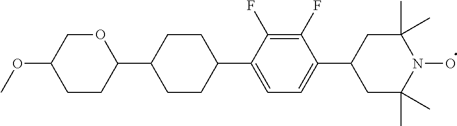 |
| (56) | 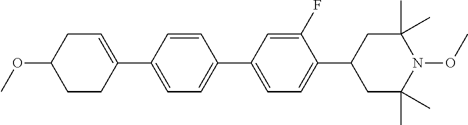 |
| (57) | 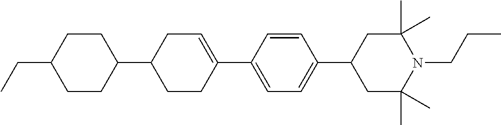 [SMILES: CCCN1C(C)(C)CC(c2ccc(C3=CCC(C4CCC(CC)CC4)CC3)cc2)CC1(C)C] |
| (58) | 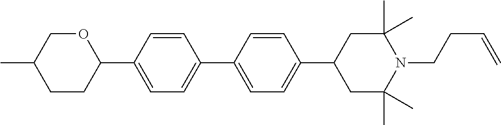 [SMILES: C=CCCN1C(C)(C)CC(c2ccc(-c3ccc(C4CCC(C)CO4)cc3)cc2)CC1(C)C] |
| (59) | 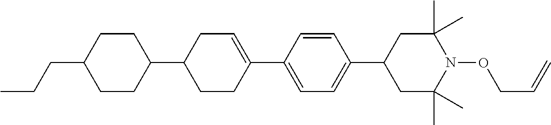 [SMILES: C=CCON1C(C)(C)CC(c2ccc(C3=CCC(C4CCC(CCC)CC4)CC3)cc2)CC1(C)C] |
| (60) | 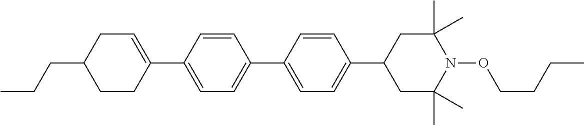 [SMILES: CCCCON1C(C)(C)CC(c2ccc(-c3ccc(C4=CCC(CCC)CC4)cc3)cc2)CC1(C)C] |
| (61) | 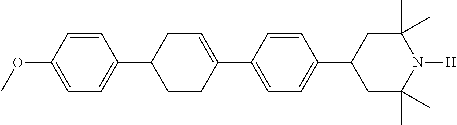 [SMILES: COc1ccc(C2CC=C(c3ccc(C4CC(C)(C)NC(C)(C)C4)cc3)CC2)cc1] |
| (62) | 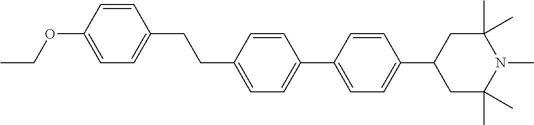 [SMILES: CCOc1ccc(CCc2ccc(-c3ccc(C4CC(C)(C)N(C)C(C)(C)C4)cc3)cc2)cc1] |
| (63) | 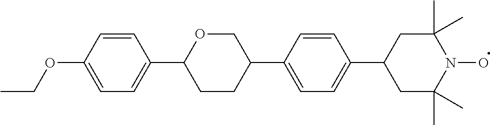 |
| (64) | 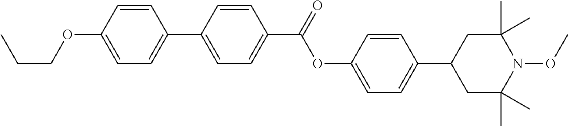 [SMILES: CCCOc1ccc(-c2ccc(C(=O)Oc3ccc(C4CC(C)(C)N(OC)C(C)(C)C4)cc3)cc2)cc1] |
| (65) | 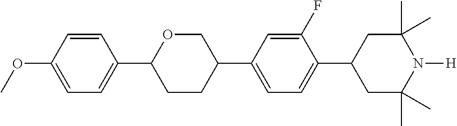 [SMILES: COc1ccc(C2CCC(c3ccc(C4CC(C)(C)NC(C)(C)C4)c(F)c3)CO2)cc1] |
| (66) | 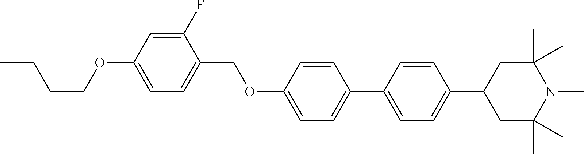 [SMILES: CCCCOc1ccc(COc2ccc(-c3ccc(C4CC(C)(C)N(C)C(C)(C)C4)cc3)cc2)c(F)c1] |
| (67) | 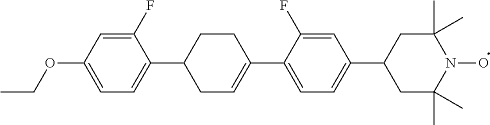 |
| (68) | 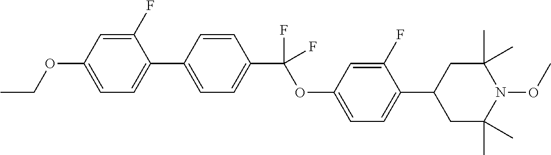 [SMILES: CCOc1ccc(-c2ccc(C(F)(F)Oc3ccc(C4CC(C)(C)N(OC)C(C)(C)C4)c(F)c3)cc2)c(F)c1] |
| (69) | 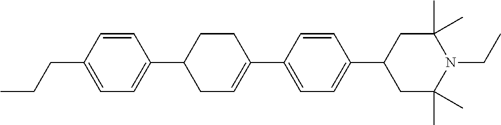 [SMILES: CCCc1ccc(C2CC=C(c3ccc(C4CC(C)(C)N(CC)C(C)(C)C4)cc3)CC2)cc1] |
| (70) | 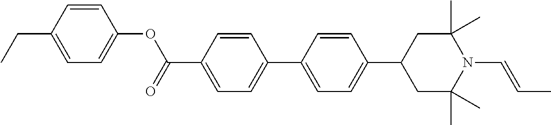 [SMILES: C/C=C/N1C(C)(C)CC(c2ccc(-c3ccc(C(=O)Oc4ccc(CC)cc4)cc3)cc2)CC1(C)C] |
| (71) | 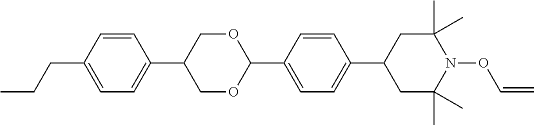 [SMILES: C=CON1C(C)(C)CC(c2ccc(C3OCC(c4ccc(CCC)cc4)CO3)cc2)CC1(C)C] |
| (72) | 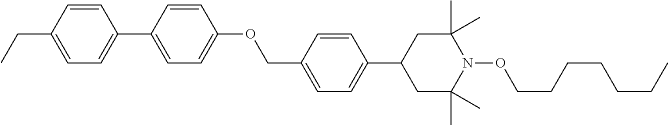 [SMILES: CCCCCCCON1C(C)(C)CC(c2ccc(COc3ccc(-c4ccc(CC)cc4)cc3)cc2)CC1(C)C] |
| (73) | 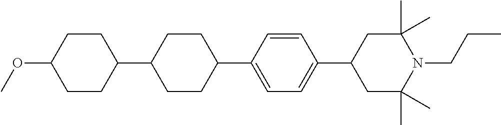 [SMILES: CCCN1C(C)(C)CC(c2ccc(C3CCC(C4CCC(OC)CC4)CC3)cc2)CC1(C)C] |
| (74) | 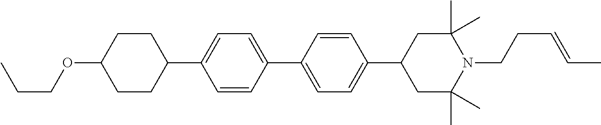 [SMILES: C/C=C/CCN1C(C)(C)CC(c2ccc(-c3ccc(C4CCC(OCCC)CC4)cc3)cc2)CC1(C)C] |
| (75) | 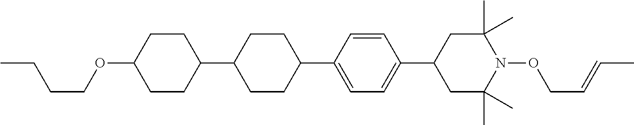 [SMILES: C/C=C/CON1C(C)(C)CC(c2ccc(C3CCC(C4CCC(OCCCC)CC4)CC3)cc2)CC1(C)C] |
| (76) | 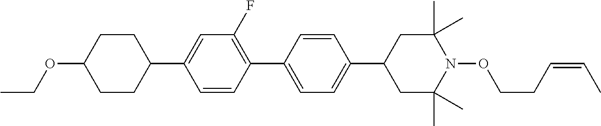 [SMILES: C/C=C\CCON1C(C)(C)CC(c2ccc(-c3ccc(C4CCC(OCC)CC4)cc3F)cc2)CC1(C)C] |
| (77) | 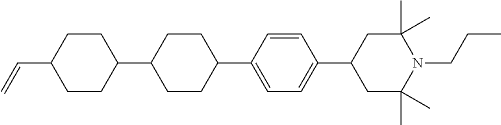 [SMILES: C=CC1CCC(C2CCC(c3ccc(C4CC(C)(C)N(CCC)C(C)(C)C4)cc3)CC2)CC1] |
| (78) | 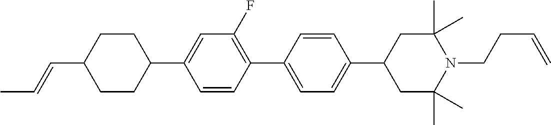 [SMILES: C=CCCN1C(C)(C)CC(c2ccc(-c3ccc(C4CCC(/C=C/C)CC4)cc3F)cc2)CC1(C)C] |
| (79) | 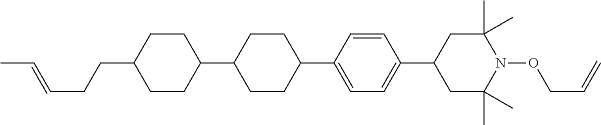 [SMILES: C=CCON1C(C)(C)CC(c2ccc(C3CCC(C4CCC(CC/C=C/C)CC4)CC3)cc2)CC1(C)C] |
| (80) | 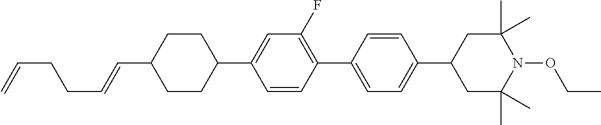 [SMILES: C=CCC/C=C/C1CCC(c2ccc(-c3ccc(C4CC(C)(C)N(OCC)C(C)(C)C4)cc3)c(F)c2)CC1] |
| (81) | 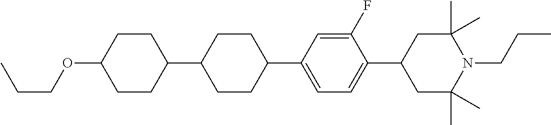 [SMILES: CCCOC1CCC(C2CCC(c3ccc(C4CC(C)(C)N(CCC)C(C)(C)C4)c(F)c3)CC2)CC1] |
| (82) | 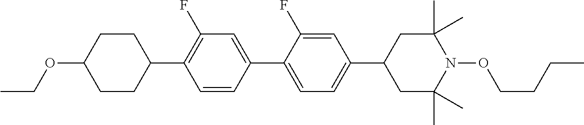 [SMILES: CCCCON1C(C)(C)CC(c2ccc(-c3ccc(C4CCC(OCC)CC4)c(F)c3)c(F)c2)CC1(C)C] |
| (83) | 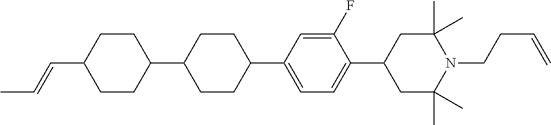 [SMILES: C=CCCN1C(C)(C)CC(c2ccc(C3CCC(C4CCC(/C=C/C)CC4)CC3)cc2F)CC1(C)C] |
| (84) | 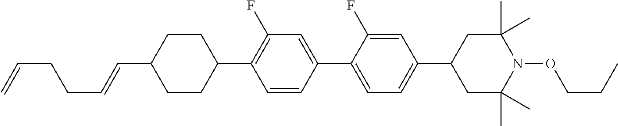 [SMILES: C=CCC/C=C/C1CCC(c2ccc(-c3ccc(C4CC(C)(C)N(OCCC)C(C)(C)C4)cc3F)cc2F)CC1] |
| (85) | 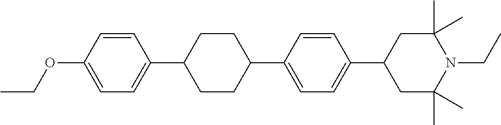 [SMILES: CCOc1ccc(C2CCC(c3ccc(C4CC(C)(C)N(CC)C(C)(C)C4)cc3)CC2)cc1] |
| (86) | 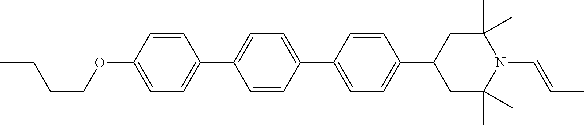 [SMILES: C/C=C/N1C(C)(C)CC(c2ccc(-c3ccc(-c4ccc(OCCCC)cc4)cc3)cc2)CC1(C)C] |
| (87) | 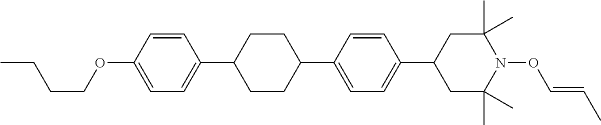 [SMILES: C/C=C/ON1C(C)(C)CC(c2ccc(C3CCC(c4ccc(OCCCC)cc4)CC3)cc2)CC1(C)C] |
| (88) | 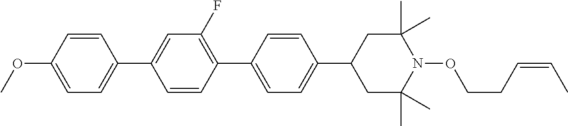 [SMILES: C/C=C\CCON1C(C)(C)CC(c2ccc(-c3ccc(-c4ccc(OC)cc4)cc3F)cc2)CC1(C)C] |
| (89) | 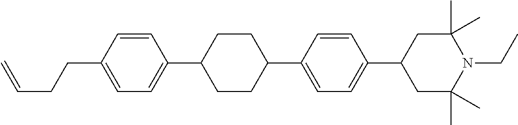 [SMILES: C=CCCc1ccc(C2CCC(c3ccc(C4CC(C)(C)N(CC)C(C)(C)C4)cc3)CC2)cc1] |
| (90) | 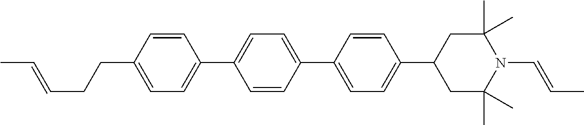 [SMILES: C/C=C/CCc1ccc(-c2ccc(-c3ccc(C4CC(C)(C)N(/C=C/C)C(C)(C)C4)cc3)cc2)cc1] |
| (91) | 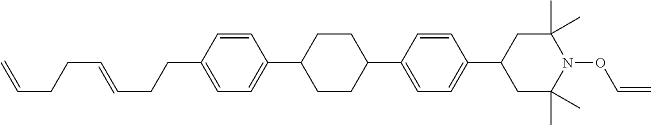 [SMILES: C=CCC/C=C/CCc1ccc(C2CCC(c3ccc(C4CC(C)(C)N(OC=C)C(C)(C)C4)cc3)CC2)cc1] |
| (92) | 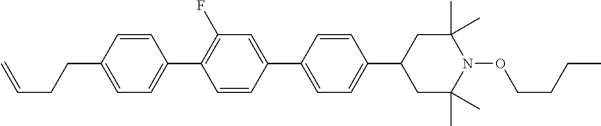 [SMILES: C=CCCc1ccc(-c2ccc(-c3ccc(C4CC(C)(C)N(OCCCC)C(C)(C)C4)cc3)cc2F)cc1] |
| (93) | 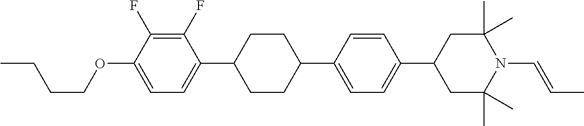 [SMILES: C/C=C/N1C(C)(C)CC(c2ccc(C3CCC(c4ccc(OCCCC)c(F)c4F)CC3)cc2)CC1(C)C] |
| (94) | 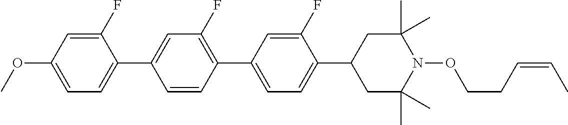 [SMILES: C/C=C\CCON1C(C)(C)CC(c2ccc(-c3ccc(-c4ccc(OC)cc4F)cc3F)cc2F)CC1(C)C] |
| (95) | 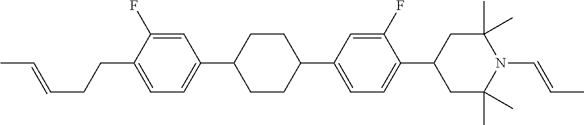 [SMILES: C/C=C/CCc1ccc(C2CCC(c3ccc(C4CC(C)(C)N(/C=C/C)C(C)(C)C4)c(F)c3)CC2)cc1F] |
| (96) | 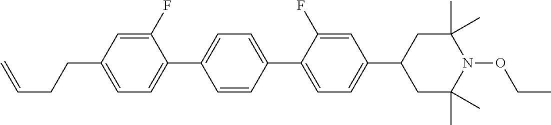 [SMILES: C=CCCc1ccc(-c2ccc(-c3ccc(C4CC(C)(C)N(OCC)C(C)(C)C4)cc3F)cc2)c(F)c1] |
| (97) | 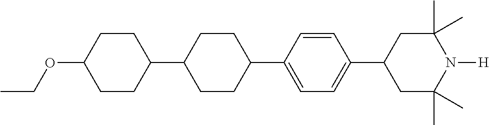 [SMILES: CCOC1CCC(C2CCC(c3ccc(C4CC(C)(C)NC(C)(C)C4)cc3)CC2)CC1] |
| (98) | 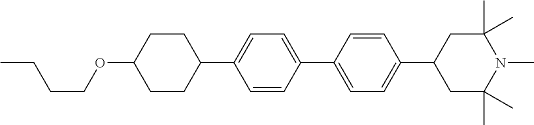 [SMILES: CCCCOC1CCC(c2ccc(-c3ccc(C4CC(C)(C)N(C)C(C)(C)C4)cc3)cc2)CC1] |
| (99) | 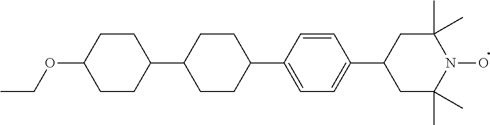 |
| (100) | 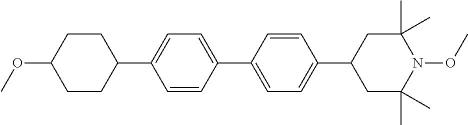 |
| (101) | 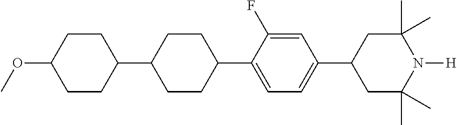 [SMILES: COC1CCC(C2CCC(c3ccc(C4CC(C)(C)NC(C)(C)C4)cc3F)CC2)CC1] |
| (102) | 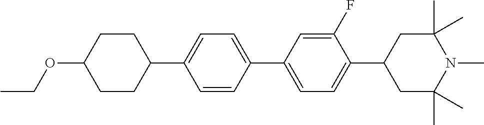 [SMILES: CCOC1CCC(c2ccc(-c3ccc(C4CC(C)(C)N(C)C(C)(C)C4)c(F)c3)cc2)CC1] |
| (103) | 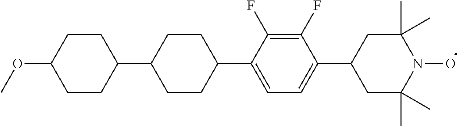 |
| (104) | 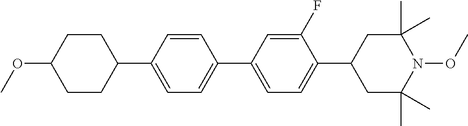 |
| (105) | 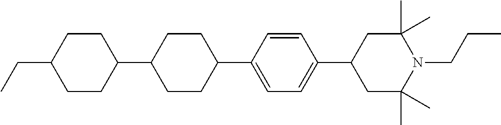 [SMILES: CCCN1C(C)(C)CC(c2ccc(C3CCC(C4CCC(CC)CC4)CC3)cc2)CC1(C)C] |
| (106) | 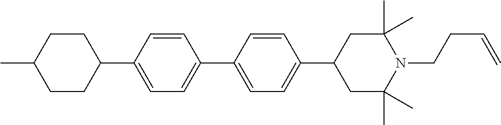 [SMILES: C=CCCN1C(C)(C)CC(c2ccc(-c3ccc(C4CCC(C)CC4)cc3)cc2)CC1(C)C] |
| (107) | 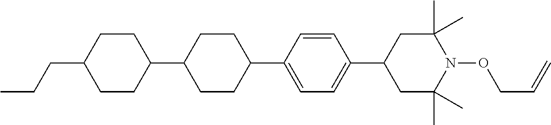 [SMILES: C=CCON1C(C)(C)CC(c2ccc(C3CCC(C4CCC(CCC)CC4)CC3)cc2)CC1(C)C] |
| (108) | 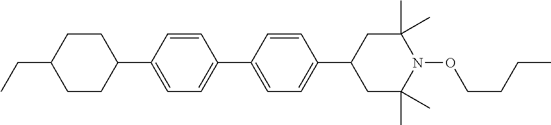 [SMILES: CCCCON1C(C)(C)CC(c2ccc(-c3ccc(C4CCC(CC)CC4)cc3)cc2)CC1(C)C] |
| (109) | 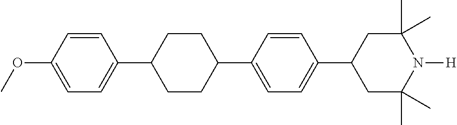 [SMILES: COc1ccc(C2CCC(c3ccc(C4CC(C)(C)NC(C)(C)C4)cc3)CC2)cc1] |
| (110) | 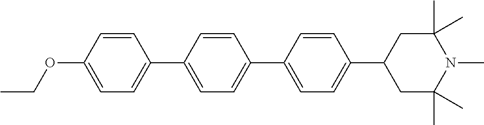 [SMILES: CCOc1ccc(-c2ccc(-c3ccc(C4CC(C)(C)N(C)C(C)(C)C4)cc3)cc2)cc1] |
| (111) | 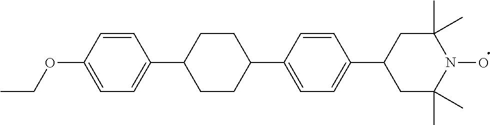 |
| (112) | 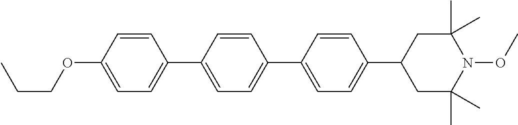 [SMILES: CCCOc1ccc(-c2ccc(-c3ccc(C4CC(C)(C)N(OC)C(C)(C)C4)cc3)cc2)cc1] |
| (113) | 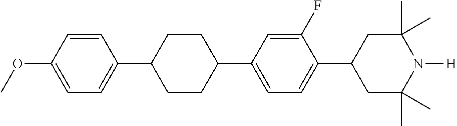 [SMILES: COc1ccc(C2CCC(c3ccc(C4CC(C)(C)NC(C)(C)C4)c(F)c3)CC2)cc1] |
| (114) | 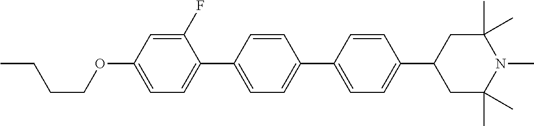 [SMILES: CCCCOc1ccc(-c2ccc(-c3ccc(C4CC(C)(C)N(C)C(C)(C)C4)cc3)cc2)c(F)c1] |
| (115) | 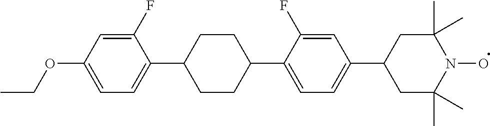 |
| (116) | 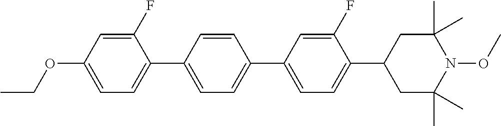 [SMILES: CCOc1ccc(-c2ccc(-c3ccc(C4CC(C)(C)N(OC)C(C)(C)C4)c(F)c3)cc2)c(F)c1] |
| (117) | 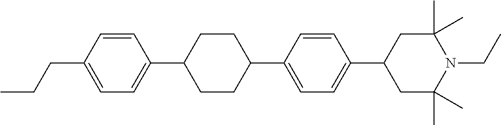 [SMILES: CCCc1ccc(C2CCC(c3ccc(C4CC(C)(C)N(CC)C(C)(C)C4)cc3)CC2)cc1] |
| (118) | 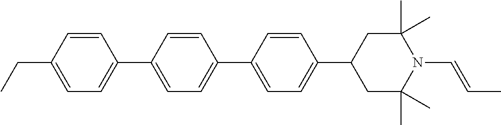 [SMILES: C/C=C/N1C(C)(C)CC(c2ccc(-c3ccc(-c4ccc(CC)cc4)cc3)cc2)CC1(C)C] |
| (119) | 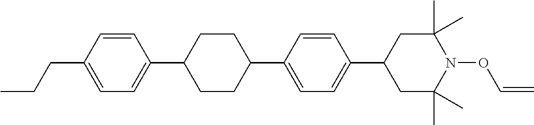 [SMILES: C=CON1C(C)(C)CC(c2ccc(C3CCC(c4ccc(CCC)cc4)CC3)cc2)CC1(C)C] |
| (120) | 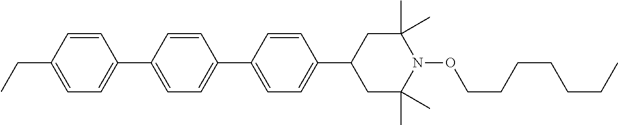 [SMILES: CCCCCCCON1C(C)(C)CC(c2ccc(-c3ccc(-c4ccc(CC)cc4)cc3)cc2)CC1(C)C] |
| (121) | 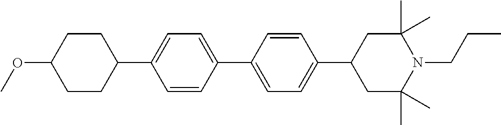 [SMILES: CCCN1C(C)(C)CC(c2ccc(-c3ccc(C4CCC(OC)CC4)cc3)cc2)CC1(C)C] |
| (122) | 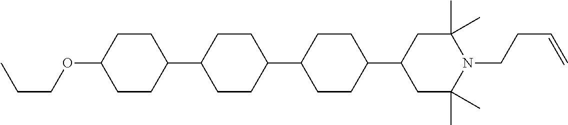 [SMILES: C=CCCN1C(C)(C)CC(C2CCC(C3CCC(C4CCC(OCCC)CC4)CC3)CC2)CC1(C)C] |
| (123) | 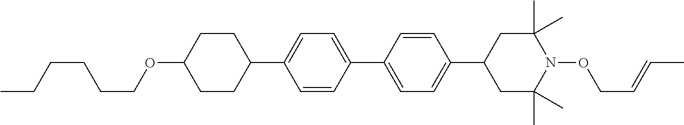 [SMILES: C/C=C/CON1C(C)(C)CC(c2ccc(-c3ccc(C4CCC(OCCCCCC)CC4)cc3)cc2)CC1(C)C] |
| (124) | 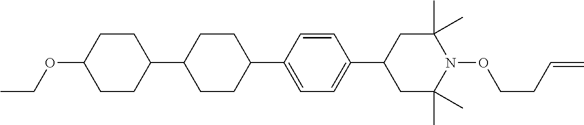 [SMILES: C=CCCON1C(C)(C)CC(c2ccc(C3CCC(C4CCC(OCC)CC4)CC3)cc2)CC1(C)C] |
| (125) | 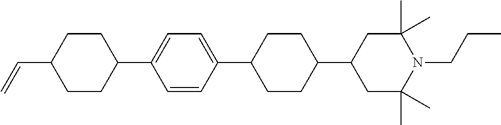 [SMILES: C=CC1CCC(c2ccc(C3CCC(C4CC(C)(C)N(CCC)C(C)(C)C4)CC3)cc2)CC1] |
| (126) | 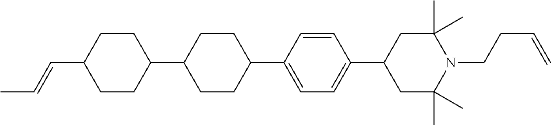 [SMILES: C=CCCN1C(C)(C)CC(c2ccc(C3CCC(C4CCC(/C=C/C)CC4)CC3)cc2)CC1(C)C] |
| (127) | 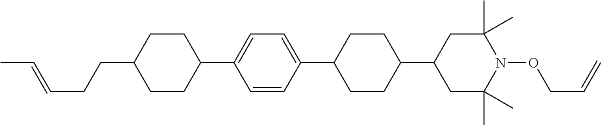 [SMILES: C=CCON1C(C)(C)CC(C2CCC(c3ccc(C4CCC(CC/C=C/C)CC4)cc3)CC2)CC1(C)C] |
| (128) | 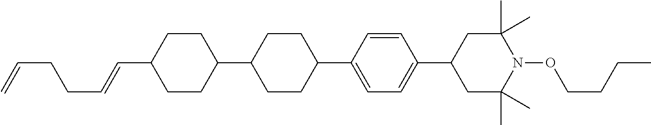 [SMILES: C=CCC/C=C/C1CCC(C2CCC(c3ccc(C4CC(C)(C)N(OCCCC)C(C)(C)C4)cc3)CC2)CC1] |
| (129) | 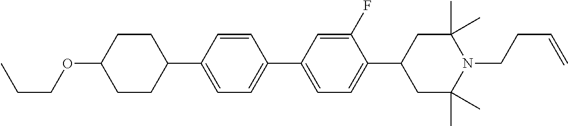 [SMILES: C=CCCN1C(C)(C)CC(c2ccc(-c3ccc(C4CCC(OCCC)CC4)cc3)cc2F)CC1(C)C] |
| (130) | 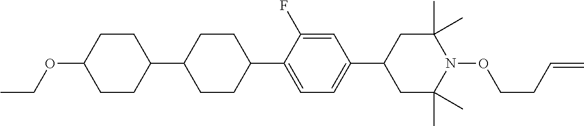 [SMILES: C=CCCON1C(C)(C)CC(c2ccc(C3CCC(C4CCC(OCC)CC4)CC3)c(F)c2)CC1(C)C] |
| (131) | 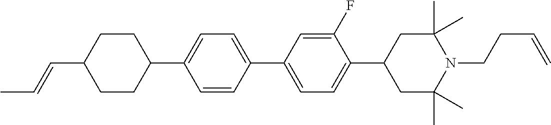 [SMILES: C=CCCN1C(C)(C)CC(c2ccc(-c3ccc(C4CCC(/C=C/C)CC4)cc3)cc2F)CC1(C)C] |
| (132) | 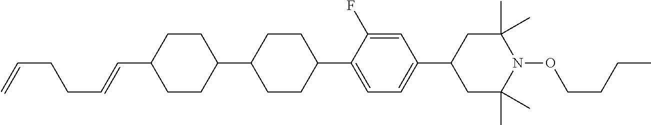 [SMILES: C=CCC/C=C/C1CCC(C2CCC(c3ccc(C4CC(C)(C)N(OCCCC)C(C)(C)C4)cc3F)CC2)CC1] |
| (133) | 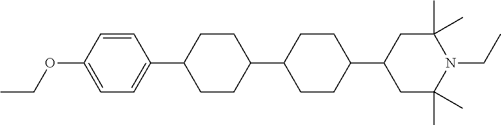 [SMILES: CCOc1ccc(C2CCC(C3CCC(C4CC(C)(C)N(CC)C(C)(C)C4)CC3)CC2)cc1] |
| (134) | 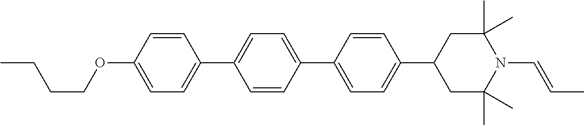 [SMILES: C/C=C/N1C(C)(C)CC(c2ccc(-c3ccc(-c4ccc(OCCCC)cc4)cc3)cc2)CC1(C)C] |
| (135) | 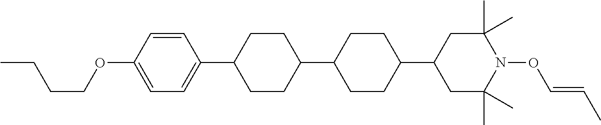 [SMILES: C/C=C/ON1C(C)(C)CC(C2CCC(C3CCC(c4ccc(OCCCC)cc4)CC3)CC2)CC1(C)C] |
| (136) | 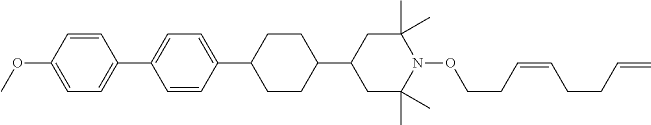 [SMILES: C=CCC/C=C\CCON1C(C)(C)CC(C2CCC(c3ccc(-c4ccc(OC)cc4)cc3)CC2)CC1(C)C] |
| (137) | 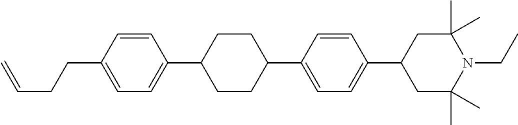 [SMILES: C=CCCc1ccc(C2CCC(c3ccc(C4CC(C)(C)N(CC)C(C)(C)C4)cc3)CC2)cc1] |
| (138) | 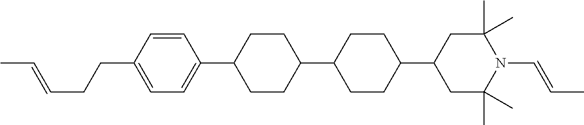 [SMILES: C/C=C/CCc1ccc(C2CCC(C3CCC(C4CC(C)(C)N(/C=C/C)C(C)(C)C4)CC3)CC2)cc1] |
| (139) | 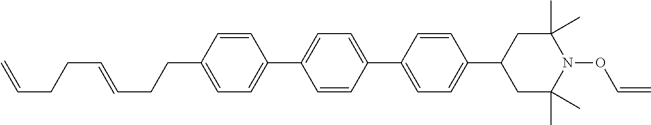 [SMILES: C=CCC/C=C/CCc1ccc(-c2ccc(-c3ccc(C4CC(C)(C)N(OC=C)C(C)(C)C4)cc3)cc2)cc1] |
| (140) | 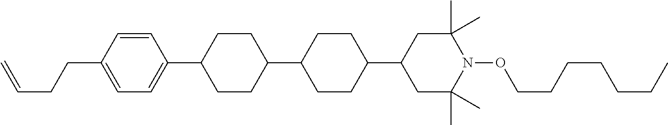 [SMILES: C=CCCc1ccc(C2CCC(C3CCC(C4CC(C)(C)N(OCCCCCCC)C(C)(C)C4)CC3)CC2)cc1] |
| (141) | 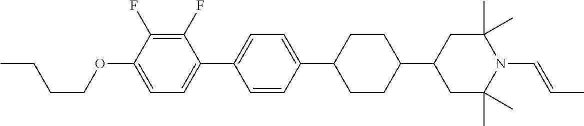 [SMILES: C/C=C/N1C(C)(C)CC(C2CCC(c3ccc(-c4ccc(OCCCC)c(F)c4F)cc3)CC2)CC1(C)C] |
| (142) | 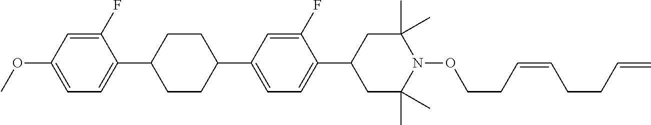 [SMILES: C=CCC/C=C\CCON1C(C)(C)CC(c2ccc(C3CCC(c4ccc(OC)cc4F)CC3)cc2F)CC1(C)C] |
| (143) | 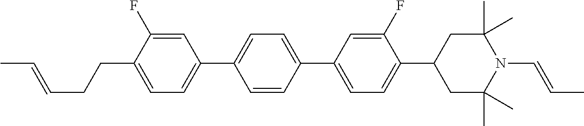 [SMILES: C/C=C/CCc1ccc(-c2ccc(-c3ccc(C4CC(C)(C)N(/C=C/C)C(C)(C)C4)c(F)c3)cc2)cc1F] |
| (144) | 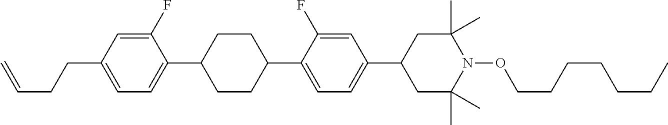 [SMILES: C=CCCc1ccc(C2CCC(c3ccc(C4CC(C)(C)N(OCCCCCCC)C(C)(C)C4)cc3F)CC2)c(F)c1] |
| (145) | 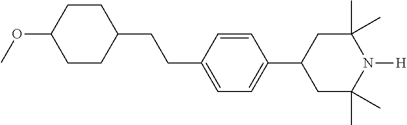 [SMILES: COC1CCC(CCc2ccc(C3CC(C)(C)NC(C)(C)C3)cc2)CC1] |
| (146) | 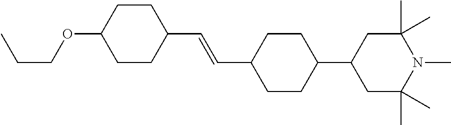 [SMILES: CCCOC1CCC(/C=C/C2CCC(C3CC(C)(C)N(C)C(C)(C)C3)CC2)CC1] |
| (147) | 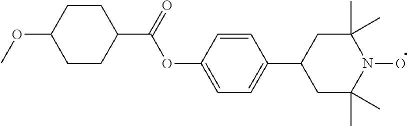 |
| (148) | 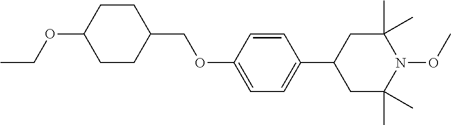 [SMILES: CCOC1CCC(COc2ccc(C3CC(C)(C)N(OC)C(C)(C)C3)cc2)CC1] |
| (149) | 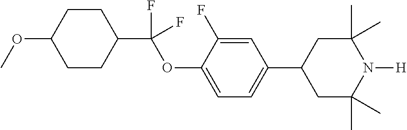 [SMILES: COC1CCC(C(F)(F)Oc2ccc(C3CC(C)(C)NC(C)(C)C3)cc2F)CC1] |
| (150) | 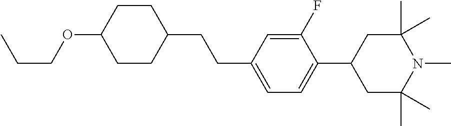 [SMILES: CCCOC1CCC(CCc2ccc(C3CC(C)(C)N(C)C(C)(C)C3)c(F)c2)CC1] |
| (151) | 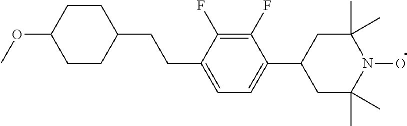 |
| (152) | 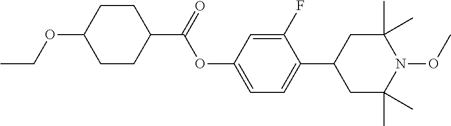 [SMILES: CCOC1CCC(C(=O)Oc2ccc(C3CC(C)(C)N(OC)C(C)(C)C3)c(F)c2)CC1] |
| (153) | 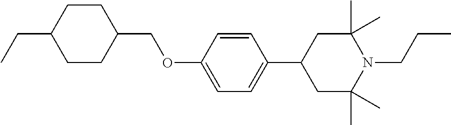 [SMILES: CCCN1C(C)(C)CC(c2ccc(OCC3CCC(CC)CC3)cc2)CC1(C)C] |
| (154) | 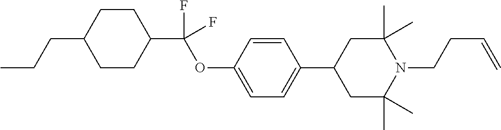 [SMILES: C=CCCN1C(C)(C)CC(c2ccc(OC(F)(F)C3CCC(CCC)CC3)cc2)CC1(C)C] |
| (155) | 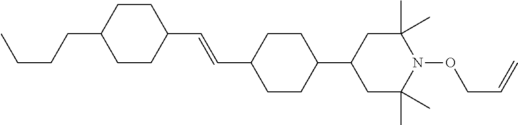 [SMILES: C=CCON1C(C)(C)CC(C2CCC(/C=C/C3CCC(CCCC)CC3)CC2)CC1(C)C] |
| (156) | 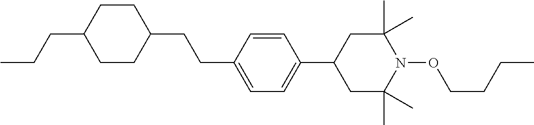 [SMILES: CCCCON1C(C)(C)CC(c2ccc(CCC3CCC(CCC)CC3)cc2)CC1(C)C] |
| (157) | 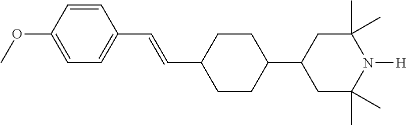 [SMILES: COc1ccc(/C=C/C2CCC(C3CC(C)(C)NC(C)(C)C3)CC2)cc1] |
| (158) | 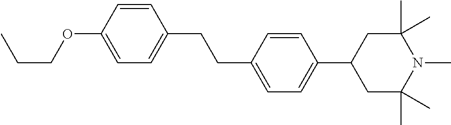 [SMILES: CCCOc1ccc(CCc2ccc(C3CC(C)(C)N(C)C(C)(C)C3)cc2)cc1] |
| (159) | 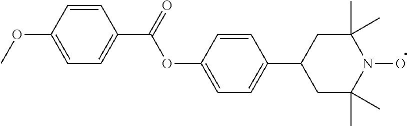 |
| (160) | 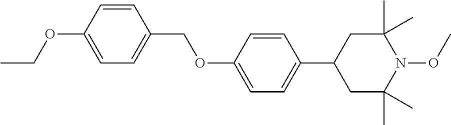 [SMILES: CCOc1ccc(COc2ccc(C3CC(C)(C)N(OC)C(C)(C)C3)cc2)cc1] |
| (161) | 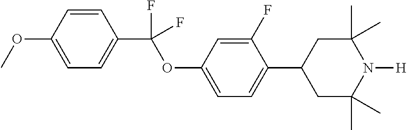 [SMILES: COc1ccc(C(F)(F)Oc2ccc(C3CC(C)(C)NC(C)(C)C3)c(F)c2)cc1] |
| (162) | 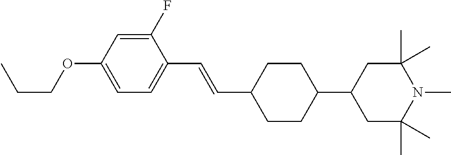 [SMILES: CCCOc1ccc(/C=C/C2CCC(C3CC(C)(C)N(C)C(C)(C)C3)CC2)c(F)c1] |
| (163) | 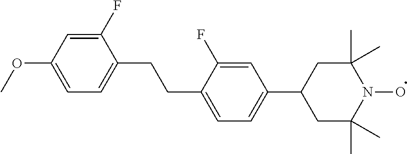 |
| (164) | 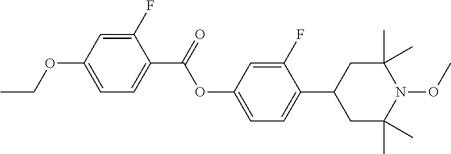 [SMILES: CCOc1ccc(C(=O)Oc2ccc(C3CC(C)(C)N(OC)C(C)(C)C3)c(F)c2)c(F)c1] |
| (165) | 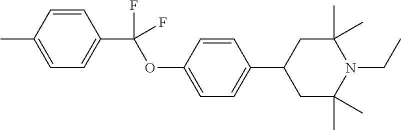 [SMILES: CCN1C(C)(C)CC(c2ccc(OC(F)(F)c3ccc(C)cc3)cc2)CC1(C)C] |
| (166) | 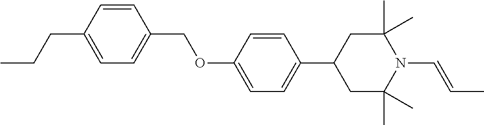 [SMILES: C/C=C/N1C(C)(C)CC(c2ccc(OCc3ccc(CCC)cc3)cc2)CC1(C)C] |
| (167) | 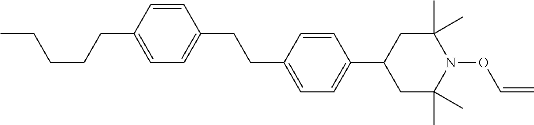 [SMILES: C=CON1C(C)(C)CC(c2ccc(CCc3ccc(CCCCC)cc3)cc2)CC1(C)C] |
| (168) | 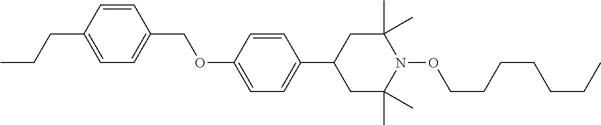 [SMILES: CCCCCCCON1C(C)(C)CC(c2ccc(OCc3ccc(CCC)cc3)cc2)CC1(C)C] |
| (169) | 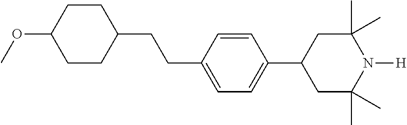 [SMILES: COC1CCC(CCc2ccc(C3CC(C)(C)NC(C)(C)C3)cc2)CC1] |
| (170) | 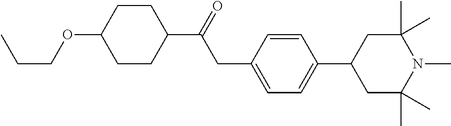 [SMILES: CCCOC1CCC(C(=O)Cc2ccc(C3CC(C)(C)N(C)C(C)(C)C3)cc2)CC1] |
| (171) | 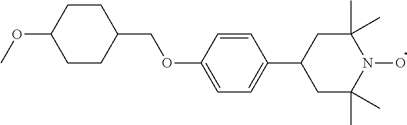 |
| (172) | 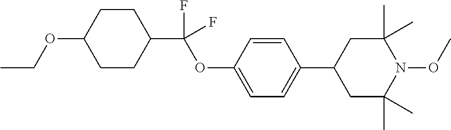 [SMILES: CCOC1CCC(C(F)(F)Oc2ccc(C3CC(C)(C)N(OC)C(C)(C)C3)cc2)CC1] |
| (173) | 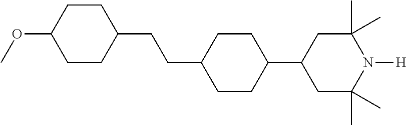 [SMILES: COC1CCC(CCC2CCC(C3CC(C)(C)NC(C)(C)C3)CC2)CC1] |
| (174) | 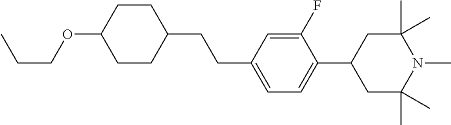 [SMILES: CCCOC1CCC(CCc2ccc(C3CC(C)(C)N(C)C(C)(C)C3)c(F)c2)CC1] |
| (175) | 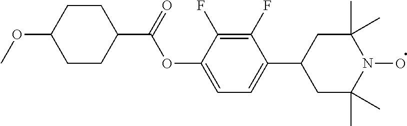 |
| (176) | 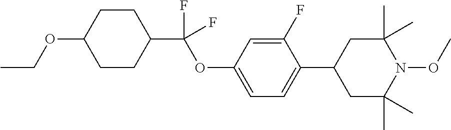 [SMILES: CCOC1CCC(C(F)(F)Oc2ccc(C3CC(C)(C)N(OC)C(C)(C)C3)c(F)c2)CC1] |
| (177) | 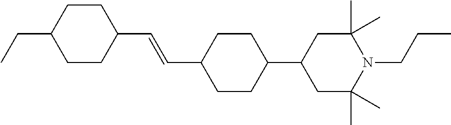 [SMILES: CCCN1C(C)(C)CC(C2CCC(/C=C/C3CCC(CC)CC3)CC2)CC1(C)C] |
| (178) | 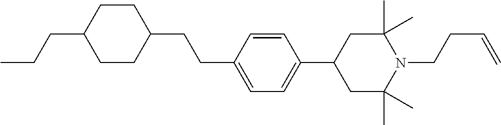 [SMILES: C=CCCN1C(C)(C)CC(c2ccc(CCC3CCC(CCC)CC3)cc2)CC1(C)C] |
| (179) | 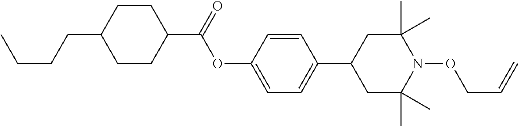 [SMILES: C=CCON1C(C)(C)CC(c2ccc(OC(=O)C3CCC(CCCC)CC3)cc2)CC1(C)C] |
| (180) | 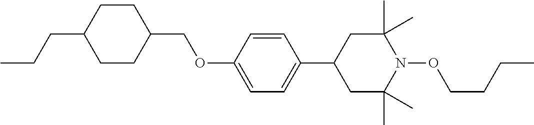 [SMILES: CCCCON1C(C)(C)CC(c2ccc(OCC3CCC(CCC)CC3)cc2)CC1(C)C] |
| (181) | 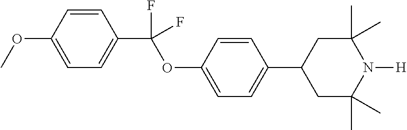 [SMILES: COc1ccc(C(F)(F)Oc2ccc(C3CC(C)(C)NC(C)(C)C3)cc2)cc1] |
| (182) | 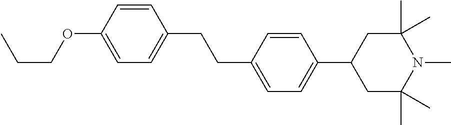 [SMILES: CCCOc1ccc(CCc2ccc(C3CC(C)(C)N(C)C(C)(C)C3)cc2)cc1] |
| (183) | 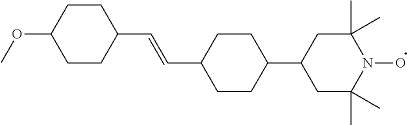 |
| (184) | 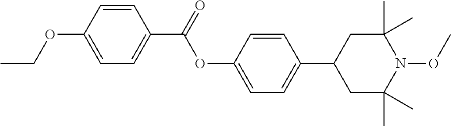 [SMILES: CCOc1ccc(C(=O)Oc2ccc(C3CC(C)(C)N(OC)C(C)(C)C3)cc2)cc1] |
| (185) | 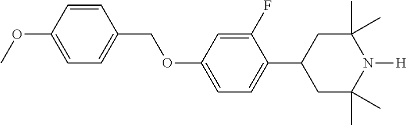 [SMILES: COc1ccc(COc2ccc(C3CC(C)(C)NC(C)(C)C3)c(F)c2)cc1] |
| (186) | 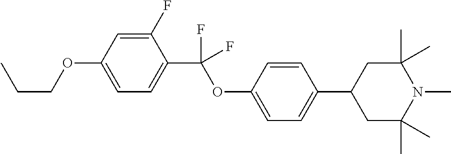 [SMILES: CCCOc1ccc(C(F)(F)Oc2ccc(C3CC(C)(C)N(C)C(C)(C)C3)cc2)c(F)c1] |
| (187) | 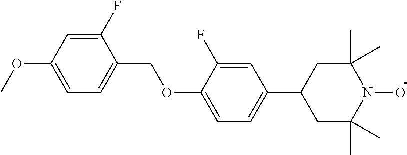 |
| (188) | 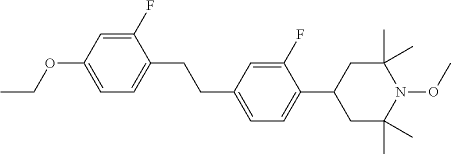 [SMILES: CCOc1ccc(CCc2ccc(C3CC(C)(C)N(OC)C(C)(C)C3)c(F)c2)c(F)c1] |
| (189) | 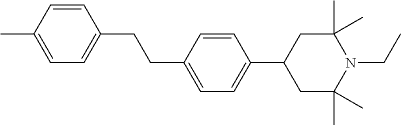 [SMILES: CCN1C(C)(C)CC(c2ccc(CCc3ccc(C)cc3)cc2)CC1(C)C] |
| (190) | 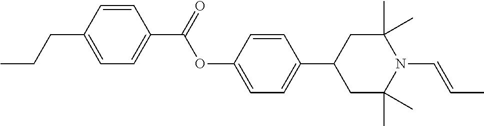 [SMILES: C/C=C/N1C(C)(C)CC(c2ccc(OC(=O)c3ccc(CCC)cc3)cc2)CC1(C)C] |
| (191) | 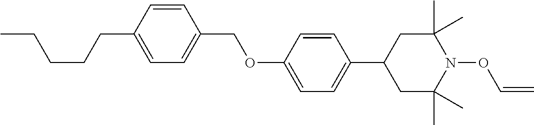 [SMILES: C=CON1C(C)(C)CC(c2ccc(OCc3ccc(CCCCC)cc3)cc2)CC1(C)C] |
| (192) | 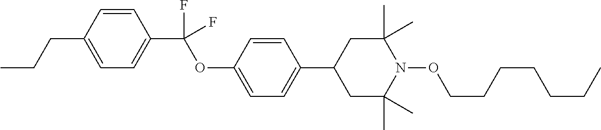 [SMILES: CCCCCCCON1C(C)(C)CC(c2ccc(OC(F)(F)c3ccc(CCC)cc3)cc2)CC1(C)C] |
| (193) | 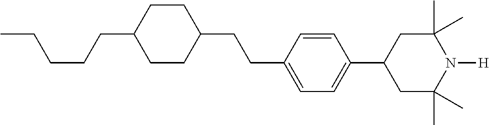 [SMILES: CCCCCC1CCC(CCc2ccc(C3CC(C)(C)NC(C)(C)C3)cc2)CC1] |
| (194) | 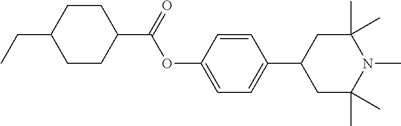 [SMILES: CCC1CCC(C(=O)Oc2ccc(C3CC(C)(C)N(C)C(C)(C)C3)cc2)CC1] |
| (195) | 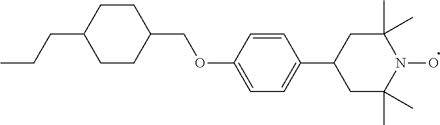 |
| (196) | 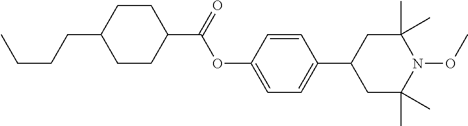 [SMILES: CCCCC1CCC(C(=O)Oc2ccc(C3CC(C)(C)N(OC)C(C)(C)C3)cc2)CC1] |
| (197) | 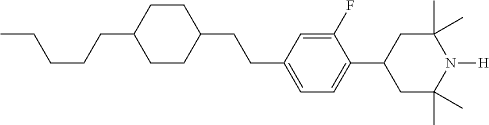 [SMILES: CCCCCC1CCC(CCc2ccc(C3CC(C)(C)NC(C)(C)C3)c(F)c2)CC1] |
| (198) | 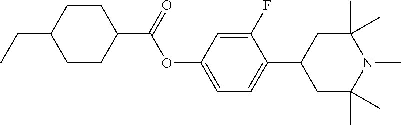 [SMILES: CCC1CCC(C(=O)Oc2ccc(C3CC(C)(C)N(C)C(C)(C)C3)c(F)c2)CC1] |
| (199) | 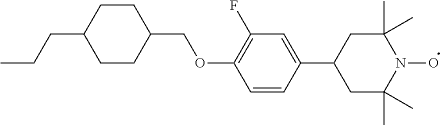 |
| (200) | 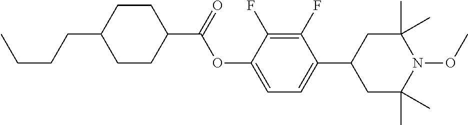 [SMILES: CCCCC1CCC(C(=O)Oc2ccc(C3CC(C)(C)N(OC)C(C)(C)C3)c(F)c2F)CC1] |
| (201) | 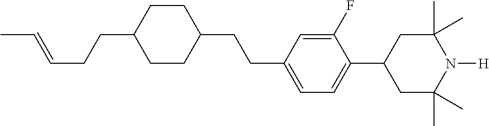 [SMILES: C/C=C/CCC1CCC(CCc2ccc(C3CC(C)(C)NC(C)(C)C3)c(F)c2)CC1] |
| (202) | 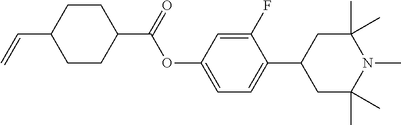 [SMILES: C=CC1CCC(C(=O)Oc2ccc(C3CC(C)(C)N(C)C(C)(C)C3)c(F)c2)CC1] |
| (203) | 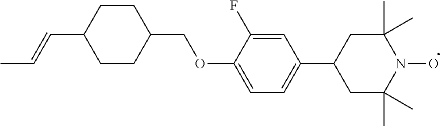 |
| (204) | 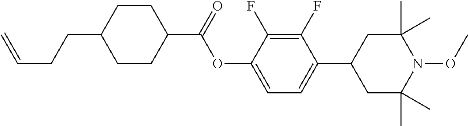 [SMILES: C=CCCC1CCC(C(=O)Oc2ccc(C3CC(C)(C)N(OC)C(C)(C)C3)c(F)c2F)CC1] |
| (205) | 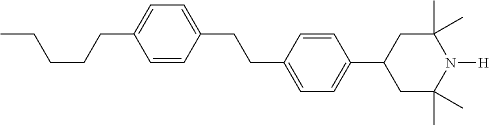 [SMILES: CCCCCc1ccc(CCc2ccc(C3CC(C)(C)NC(C)(C)C3)cc2)cc1] |
| (206) | 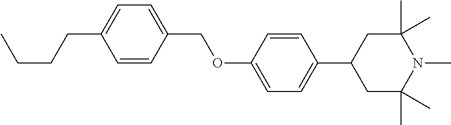 [SMILES: CCCCc1ccc(COc2ccc(C3CC(C)(C)N(C)C(C)(C)C3)cc2)cc1] |
| (207) | 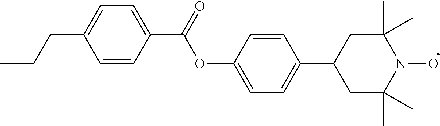 |
| (208) | 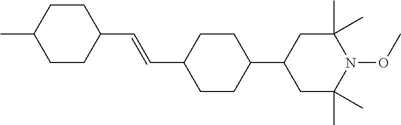 |
| (209) | 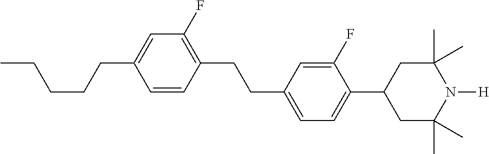 [SMILES: CCCCCc1ccc(CCc2ccc(C3CC(C)(C)NC(C)(C)C3)c(F)c2)c(F)c1] |
| (210) | 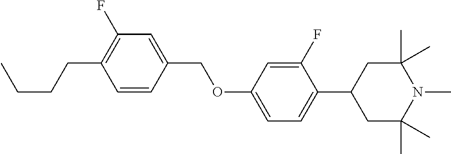 [SMILES: CCCCc1ccc(COc2ccc(C3CC(C)(C)N(C)C(C)(C)C3)c(F)c2)cc1F] |
| (211) | 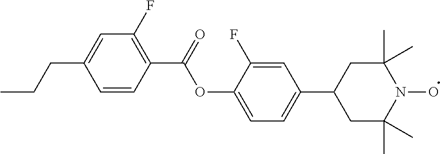 |
| (212) | 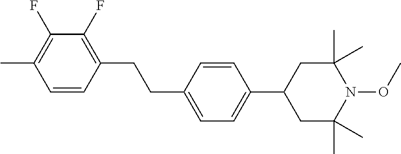 |
| (213) | 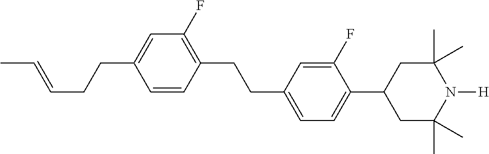 [SMILES: C/C=C/CCc1ccc(CCc2ccc(C3CC(C)(C)NC(C)(C)C3)c(F)c2)c(F)c1] |
| (214) | 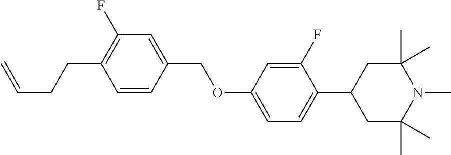 [SMILES: C=CCCc1ccc(COc2ccc(C3CC(C)(C)N(C)C(C)(C)C3)c(F)c2)cc1F] |
| (215) | 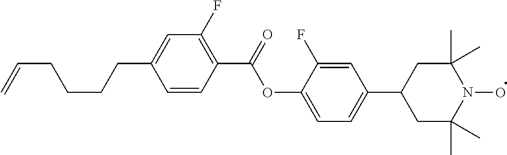 |
| (216) | 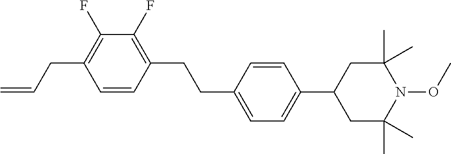 [SMILES: C=CCc1ccc(CCc2ccc(C3CC(C)(C)N(OC)C(C)(C)C3)cc2)c(F)c1F] |
| (217) | 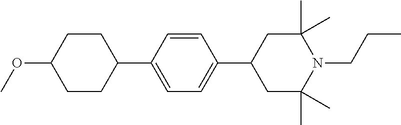 [SMILES: CCCN1C(C)(C)CC(c2ccc(C3CCC(OC)CC3)cc2)CC1(C)C] |
| (218) | 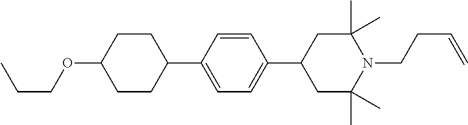 [SMILES: C=CCCN1C(C)(C)CC(c2ccc(C3CCC(OCCC)CC3)cc2)CC1(C)C] |
| (219) | 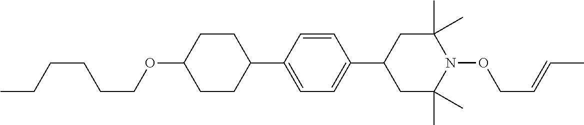 [SMILES: C/C=C/CON1C(C)(C)CC(c2ccc(C3CCC(OCCCCCC)CC3)cc2)CC1(C)C] |
| (220) | 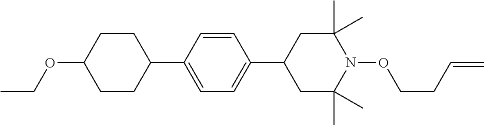 [SMILES: C=CCCON1C(C)(C)CC(c2ccc(C3CCC(OCC)CC3)cc2)CC1(C)C] |
| (221) | 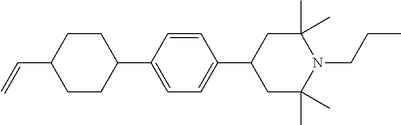 [SMILES: C=CC1CCC(c2ccc(C3CC(C)(C)N(CCC)C(C)(C)C3)cc2)CC1] |
| (222) | 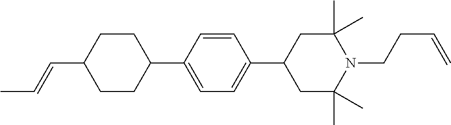 [SMILES: C=CCCN1C(C)(C)CC(c2ccc(C3CCC(/C=C/C)CC3)cc2)CC1(C)C] |
| (223) | 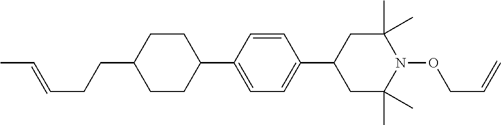 [SMILES: C=CCON1C(C)(C)CC(c2ccc(C3CCC(CC/C=C/C)CC3)cc2)CC1(C)C] |
| (224) | 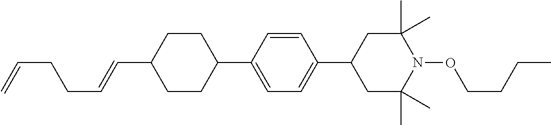 [SMILES: C=CCC/C=C/C1CCC(c2ccc(C3CC(C)(C)N(OCCCC)C(C)(C)C3)cc2)CC1] |
| (225) | 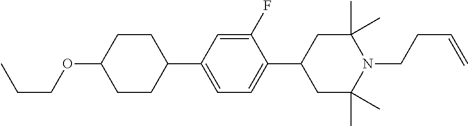 [SMILES: C=CCCN1C(C)(C)CC(c2ccc(C3CCC(OCCC)CC3)cc2F)CC1(C)C] |
| (226) | 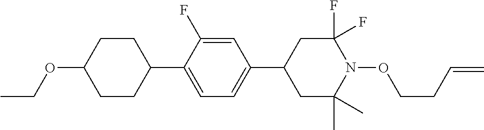 [SMILES: C=CCCON1C(C)(C)CC(c2ccc(C3CCC(OCC)CC3)c(F)c2)CC1(F)F] |
| (227) | 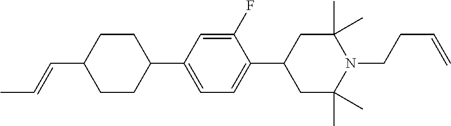 [SMILES: C=CCCN1C(C)(C)CC(c2ccc(C3CCC(/C=C/C)CC3)cc2F)CC1(C)C] |
| (228) | 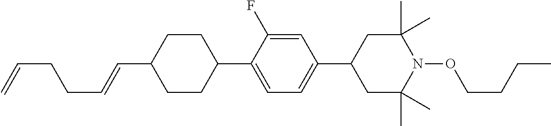 [SMILES: C=CCC/C=C/C1CCC(c2ccc(C3CC(C)(C)N(OCCCC)C(C)(C)C3)cc2F)CC1] |
| (229) | 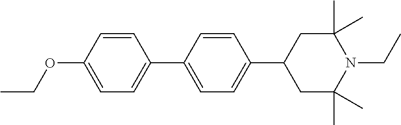 [SMILES: CCOc1ccc(-c2ccc(C3CC(C)(C)N(CC)C(C)(C)C3)cc2)cc1] |
| (230) | 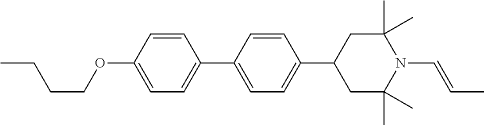 [SMILES: C/C=C/N1C(C)(C)CC(c2ccc(-c3ccc(OCCCC)cc3)cc2)CC1(C)C] |
| (231) | 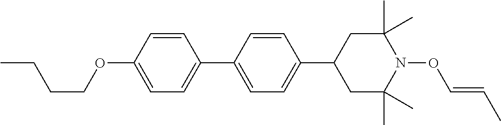 [SMILES: C/C=C/ON1C(C)(C)CC(c2ccc(-c3ccc(OCCCC)cc3)cc2)CC1(C)C] |
| (232) | 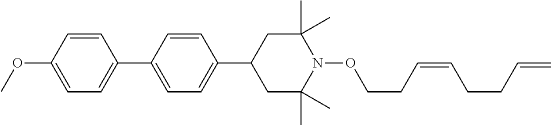 [SMILES: C=CCC/C=C\CCON1C(C)(C)CC(c2ccc(-c3ccc(OC)cc3)cc2)CC1(C)C] |
| (233) | 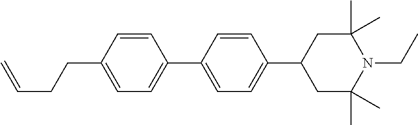 [SMILES: C=CCCc1ccc(-c2ccc(C3CC(C)(C)N(CC)C(C)(C)C3)cc2)cc1] |
| (234) | 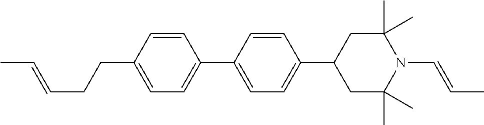 [SMILES: C/C=C/CCc1ccc(-c2ccc(C3CC(C)(C)N(/C=C/C)C(C)(C)C3)cc2)cc1] |
| (235) | 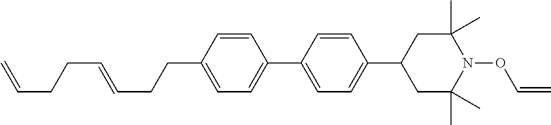 [SMILES: C=CCC/C=C/CCc1ccc(-c2ccc(C3CC(C)(C)N(OC=C)C(C)(C)C3)cc2)cc1] |
| (236) | 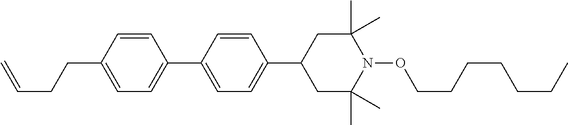 [SMILES: C=CCCc1ccc(-c2ccc(C3CC(C)(C)N(OCCCCCCC)C(C)(C)C3)cc2)cc1] |
| (237) | 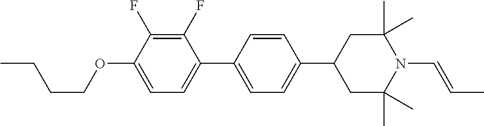 [SMILES: C/C=C/N1C(C)(C)CC(c2ccc(-c3ccc(OCCCC)c(F)c3F)cc2)CC1(C)C] |
| (238) | 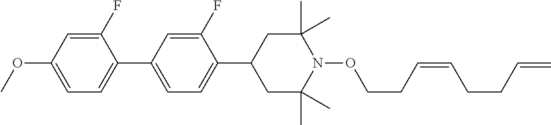 [SMILES: C=CCC/C=C\CCON1C(C)(C)CC(c2ccc(-c3ccc(OC)cc3F)cc2F)CC1(C)C] |
| (239) | 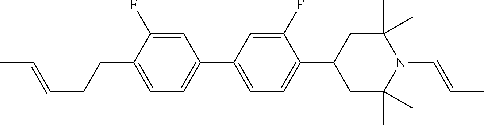 [SMILES: C/C=C/CCc1ccc(-c2ccc(C3CC(C)(C)N(/C=C/C)C(C)(C)C3)c(F)c2)cc1F] |
| (240) | 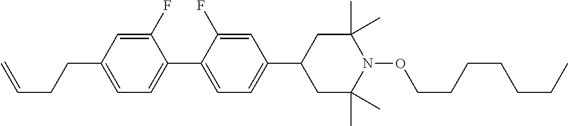 [SMILES: C=CCCc1ccc(-c2ccc(C3CC(C)(C)N(OCCCCCCC)C(C)(C)C3)cc2F)c(F)c1] |
| (241) | 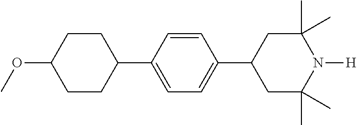 [SMILES: COC1CCC(c2ccc(C3CC(C)(C)NC(C)(C)C3)cc2)CC1] |
| (242) | 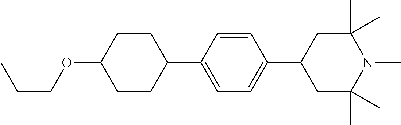 [SMILES: CCCOC1CCC(c2ccc(C3CC(C)(C)N(C)C(C)(C)C3)cc2)CC1] |
| (243) | 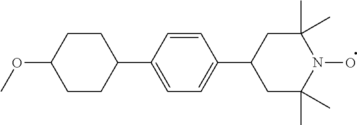 |
| (244) | 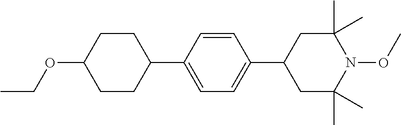 [SMILES: CCOC1CCC(c2ccc(C3CC(C)(C)N(OC)C(C)(C)C3)cc2)CC1] |
| (245) | 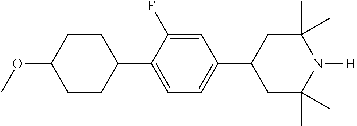 [SMILES: COC1CCC(c2ccc(C3CC(C)(C)NC(C)(C)C3)cc2F)CC1] |
| (246) | 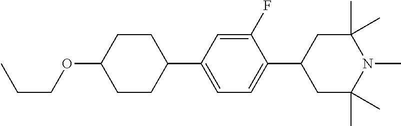 [SMILES: CCCOC1CCC(c2ccc(C3CC(C)(C)N(C)C(C)(C)C3)c(F)c2)CC1] |
| (247) | 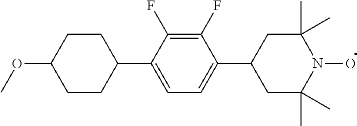 |
| (248) | 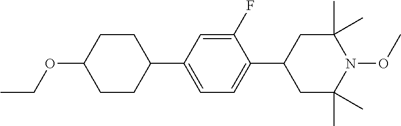 [SMILES: CCOC1CCC(c2ccc(C3CC(C)(C)N(OC)C(C)(C)C3)c(F)c2)CC1] |
| (249) | 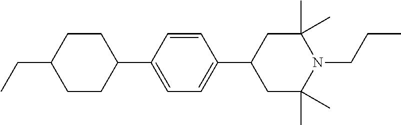 [SMILES: CCCN1C(C)(C)CC(c2ccc(C3CCC(CC)CC3)cc2)CC1(C)C] |
| (250) | 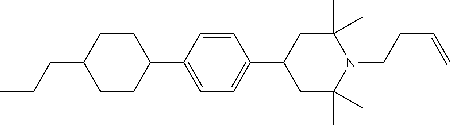 [SMILES: C=CCCN1C(C)(C)CC(c2ccc(C3CCC(CCC)CC3)cc2)CC1(C)C] |
| (251) | 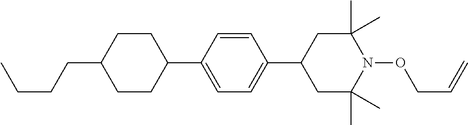 [SMILES: C=CCON1C(C)(C)CC(c2ccc(C3CCC(CCCC)CC3)cc2)CC1(C)C] |
| (252) | 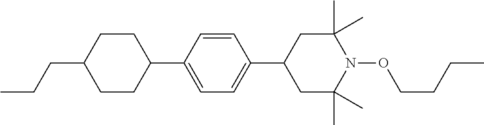 [SMILES: CCCCON1C(C)(C)CC(c2ccc(C3CCC(CCC)CC3)cc2)CC1(C)C] |
| (253) | 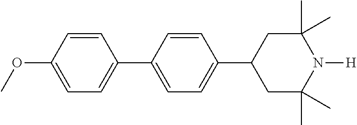 [SMILES: COc1ccc(-c2ccc(C3CC(C)(C)NC(C)(C)C3)cc2)cc1] |
| (254) | 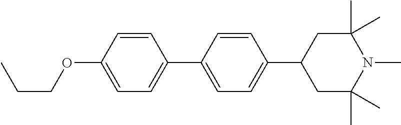 [SMILES: CCCOc1ccc(-c2ccc(C3CC(C)(C)N(C)C(C)(C)C3)cc2)cc1] |
| (255) | 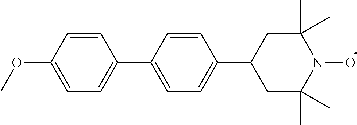 |
| (256) | 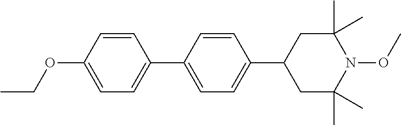 [SMILES: CCOc1ccc(-c2ccc(C3CC(C)(C)N(OC)C(C)(C)C3)cc2)cc1] |
| (257) | 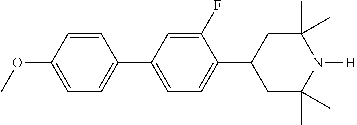 [SMILES: COc1ccc(-c2ccc(C3CC(C)(C)NC(C)(C)C3)c(F)c2)cc1] |
| (258) | 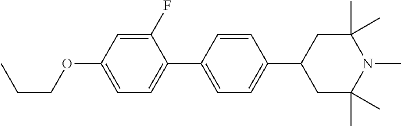 [SMILES: CCCOc1ccc(-c2ccc(C3CC(C)(C)N(C)C(C)(C)C3)cc2)c(F)c1] |
| (259) | 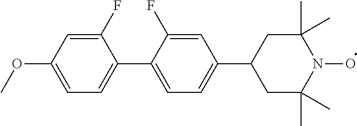 |
| (260) | 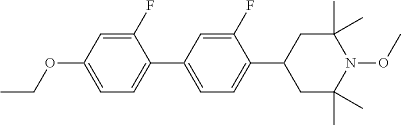 [SMILES: CCOc1ccc(-c2ccc(C3CC(C)(C)N(OC)C(C)(C)C3)c(F)c2)c(F)c1] |
| (261) | 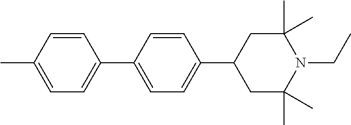 [SMILES: CCN1C(C)(C)CC(c2ccc(-c3ccc(C)cc3)cc2)CC1(C)C] |
| (262) | 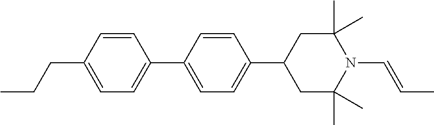 [SMILES: C/C=C/N1C(C)(C)CC(c2ccc(-c3ccc(CCC)cc3)cc2)CC1(C)C] |
| (263) | 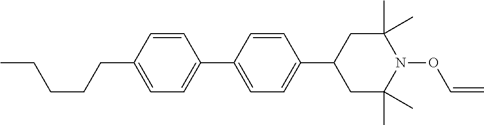 [SMILES: C=CON1C(C)(C)CC(c2ccc(-c3ccc(CCCCC)cc3)cc2)CC1(C)C] |
| (264) | 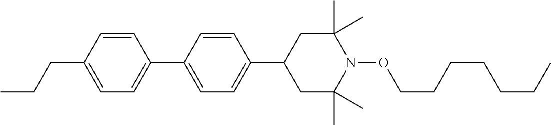 [SMILES: CCCCCCCON1C(C)(C)CC(c2ccc(-c3ccc(CCC)cc3)cc2)CC1(C)C] |
| (265) | 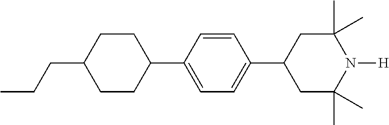 [SMILES: CCCC1CCC(c2ccc(C3CC(C)(C)NC(C)(C)C3)cc2)CC1] |
| (266) | 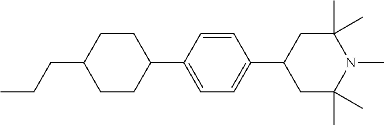 [SMILES: CCCC1CCC(c2ccc(C3CC(C)(C)N(C)C(C)(C)C3)cc2)CC1] |
| (267) | 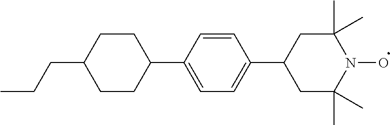 |
| (268) | 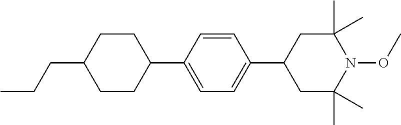 [SMILES: CCCC1CCC(c2ccc(C3CC(C)(C)N(OC)C(C)(C)C3)cc2)CC1] |
| (269) | 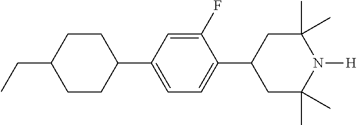 [SMILES: CCC1CCC(c2ccc(C3CC(C)(C)NC(C)(C)C3)c(F)c2)CC1] |
| (270) | 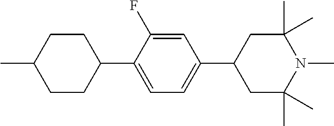 |
| (271) | 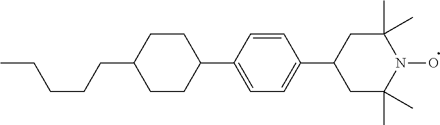 |
| (272) | 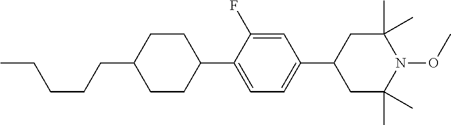 [SMILES: CCCCCC1CCC(c2ccc(C3CC(C)(C)N(OC)C(C)(C)C3)cc2F)CC1] |
| (273) | 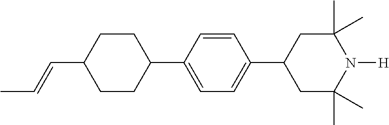 [SMILES: C/C=C/C1CCC(c2ccc(C3CC(C)(C)NC(C)(C)C3)cc2)CC1] |
| (274) | 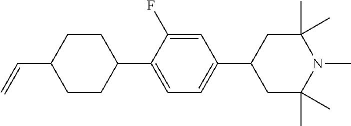 [SMILES: C=CC1CCC(c2ccc(C3CC(C)(C)N(C)C(C)(C)C3)cc2F)CC1] |
| (275) | 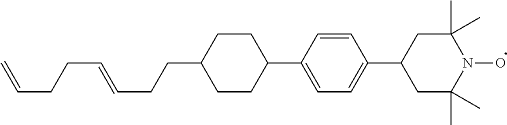 |
| (276) | 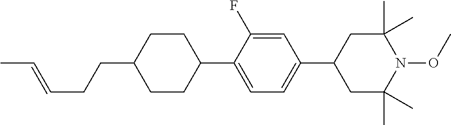 [SMILES: C/C=C/CCC1CCC(c2ccc(C3CC(C)(C)N(OC)C(C)(C)C3)cc2F)CC1] |
| (277) | 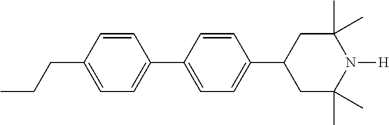 [SMILES: CCCc1ccc(-c2ccc(C3CC(C)(C)NC(C)(C)C3)cc2)cc1] |
| (278) | 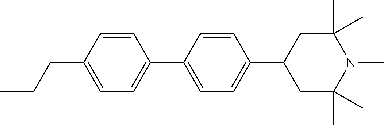 [SMILES: CCCc1ccc(-c2ccc(C3CC(C)(C)N(C)C(C)(C)C3)cc2)cc1] |
| (279) | 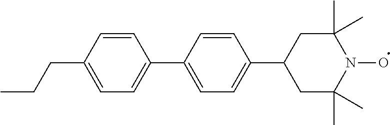 |
| (280) | 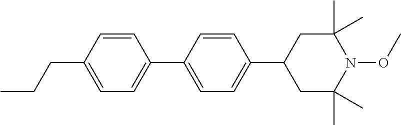 [SMILES: CCCc1ccc(-c2ccc(C3CC(C)(C)N(OC)C(C)(C)C3)cc2)cc1] |
| (281) | 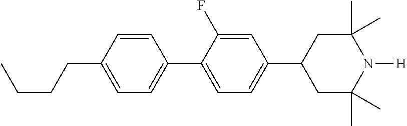 [SMILES: CCCCc1ccc(-c2ccc(C3CC(C)(C)NC(C)(C)C3)cc2F)cc1] |
| (282) | 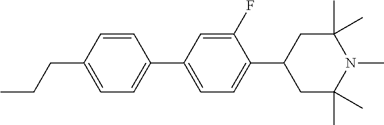 [SMILES: CCCc1ccc(-c2ccc(C3CC(C)(C)N(C)C(C)(C)C3)c(F)c2)cc1] |
| (283) | 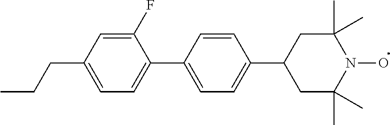 |
| (284) | 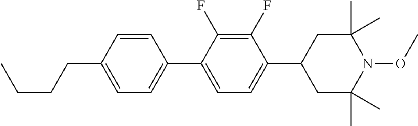 [SMILES: CCCCc1ccc(-c2ccc(C3CC(C)(C)N(OC)C(C)(C)C3)c(F)c2F)cc1] |
| (285) | 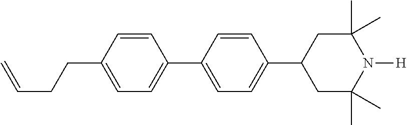 [SMILES: C=CCCc1ccc(-c2ccc(C3CC(C)(C)NC(C)(C)C3)cc2)cc1] |
| (286) | 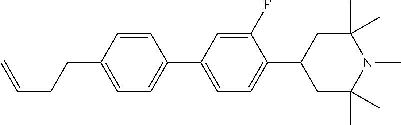 [SMILES: C=CCCc1ccc(-c2ccc(C3CC(C)(C)N(C)C(C)(C)C3)c(F)c2)cc1] |
| (287) | 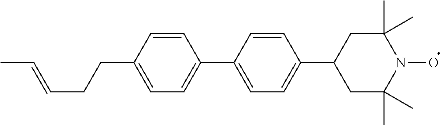 |
| (288) | 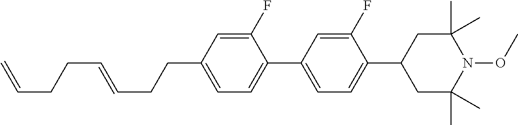 [SMILES: C=CCC/C=C/CCc1ccc(-c2ccc(C3CC(C)(C)N(OC)C(C)(C)C3)c(F)c2)c(F)c1] |
| (289) | 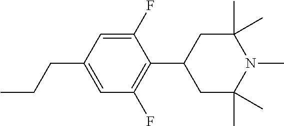 [SMILES: CCCc1cc(F)c(C2CC(C)(C)N(C)C(C)(C)C2)c(F)c1] |
| (290) | 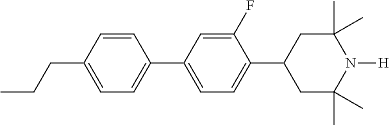 [SMILES: CCCc1ccc(-c2ccc(C3CC(C)(C)NC(C)(C)C3)c(F)c2)cc1] |
| (291) | 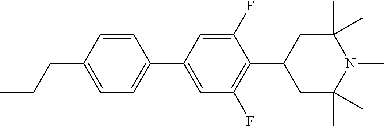 [SMILES: CCCc1ccc(-c2cc(F)c(C3CC(C)(C)N(C)C(C)(C)C3)c(F)c2)cc1] |
| (292) | 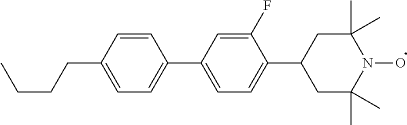 |
| (293) | 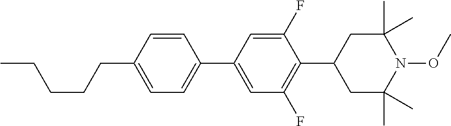 [SMILES: CCCCCc1ccc(-c2cc(F)c(C3CC(C)(C)N(OC)C(C)(C)C3)c(F)c2)cc1] |
| (294) | 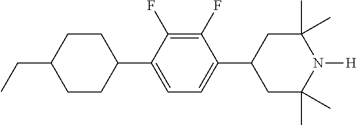 [SMILES: CCC1CCC(c2ccc(C3CC(C)(C)NC(C)(C)C3)c(F)c2F)CC1] |
| (295) | 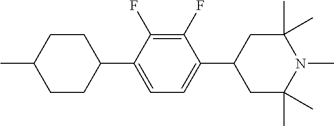 |
| (296) | 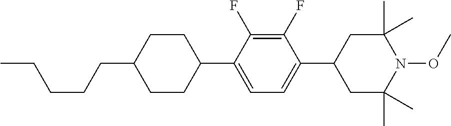 [SMILES: CCCCCC1CCC(c2ccc(C3CC(C)(C)N(OC)C(C)(C)C3)c(F)c2F)CC1] |
| (297) | 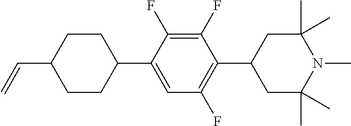 [SMILES: C=CC1CCC(c2cc(F)c(C3CC(C)(C)N(C)C(C)(C)C3)c(F)c2F)CC1] |
| (298) | 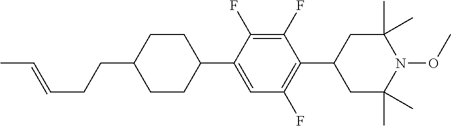 [SMILES: C/C=C/CCC1CCC(c2cc(F)c(C3CC(C)(C)N(OC)C(C)(C)C3)c(F)c2F)CC1] |
| (299) | 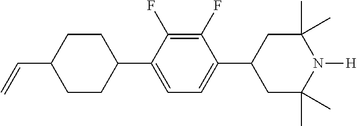 [SMILES: C=CC1CCC(c2ccc(C3CC(C)(C)NC(C)(C)C3)c(F)c2F)CC1] |
| (300) | 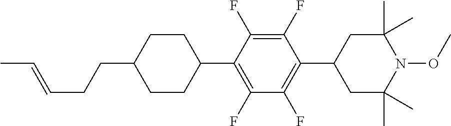 [SMILES: C/C=C/CCC1CCC(c2c(F)c(F)c(C3CC(C)(C)N(OC)C(C)(C)C3)c(F)c2F)CC1] |
| (301) | 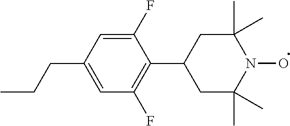 |
| (302) | 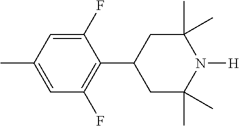 [SMILES: Cc1cc(F)c(C2CC(C)(C)NC(C)(C)C2)c(F)c1] |
| (303) | 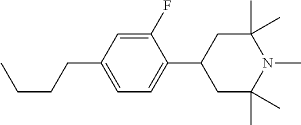 [SMILES: CCCCc1ccc(C2CC(C)(C)N(C)C(C)(C)C2)c(F)c1] |
| (304) | 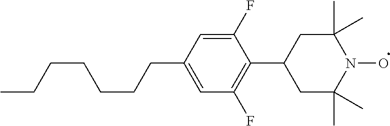 |
| (305) | 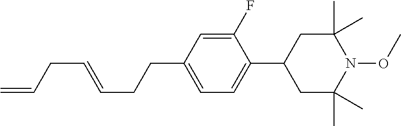 [SMILES: C=CC/C=C/CCc1ccc(C2CC(C)(C)N(OC)C(C)(C)C2)c(F)c1] |
| (306) | 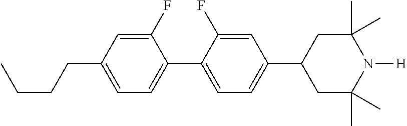 [SMILES: CCCCc1ccc(-c2ccc(C3CC(C)(C)NC(C)(C)C3)cc2F)c(F)c1] |
| (307) | 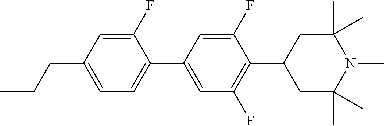 [SMILES: CCCc1ccc(-c2cc(F)c(C3CC(C)(C)N(C)C(C)(C)C3)c(F)c2)c(F)c1] |
| (308) | 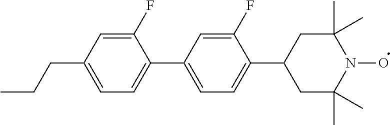 |
| (309) | 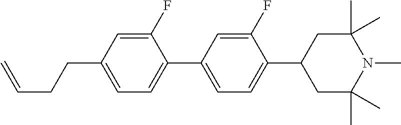 [SMILES: C=CCCc1ccc(-c2ccc(C3CC(C)(C)N(C)C(C)(C)C3)c(F)c2)c(F)c1] |
| (310) | 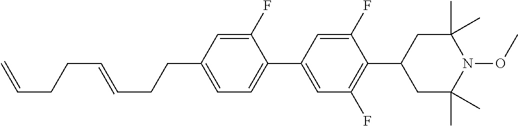 [SMILES: C=CCC/C=C/CCc1ccc(-c2cc(F)c(C3CC(C)(C)N(OC)C(C)(C)C3)c(F)c2)c(F)c1] |
| (311) | 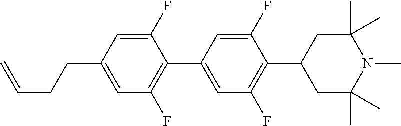 [SMILES: C=CCCc1cc(F)c(-c2cc(F)c(C3CC(C)(C)N(C)C(C)(C)C3)c(F)c2)c(F)c1] |
| (312) | 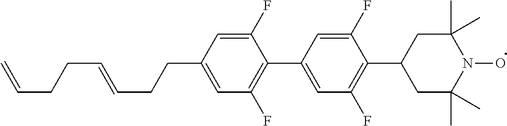 |

### 2. Examples of Liquid Crystal Composition

The invention will be described in greater detail by way of Use Examples. However, the invention is not limited by the Use Examples. The invention includes a mixture of a composition in Use Example 1 and a composition in Use Example 2. The invention also includes a mixture in which at least two compositions in Use Examples are mixed. Compounds in Examples (including Use Examples) are represented using symbols according to definitions in Table 3 described below. In Table 3, a configuration with regard to 1,4-cyclohexylene is trans. A parenthesized number next to a symbol in Examples represents a chemical formula to which the compound belongs. A symbol (-) means any other liquid crystal compound. A proportion of the liquid crystal compound is expressed in terms of weight percent (% by weight) based thereon. Values of the characteristics of the liquid crystal composition are summarized in a last part. Characteristics were measured according to the methods described above, and measured values were directly described (without extrapolation).

TABLE 3
Method for description of compounds using symbols
R—(A₁)—Z₁— . . . -Zₙ—(Aₙ)—R′

| 1) Left-terminal group R— | Symbol |
|---|---|
| CₙH₂ₙ₊₁— | n— |
| CₙH₂ₙ₊₁O— | nO— |
| CₘH₂ₘ₊₁OCₙH₂ₙ— | mOn— |
| CH₂═CH— | V— |
| CₙH₂ₙ₊₁—CH═CH— | nV— |
| CH₂═CH—CₙH₂ₙ— | Vn— |
| CₘH₂ₘ₊₁—CH═CH—CₙH₂ₙ— | mVn— |
| CF₂═CH— | VFF— |
| CF₂═CH—CₙH₂ₙ— | VFFn— |
| 2) Right-terminal group —R′ Symbol | |
| —CₙH₂ₙ₊₁ | —n |
| —OCₙH₂ₙ₊₁ | —On |
| —COOCH₃ | —EMe |
| —CH═CH₂ | —V |
| —CH═CH—CₙH₂ₙ₊₁ | —Vn |
| —CₙH₂ₙ—CH=CH₂ | —nV |
| —CₘH₂ₘ—CH═CH—CₙH₂ₙ₊₁ | —mVn |
| —CH═CF₂ | —VFF |
| —F | —F |
| —Cl | —CL |
| —OCF₃ | —OCF3 |
| —OCF₂H | —OCF2H |
| —CF₃ | —CF3 |
| —OCH═CH—CF₃ | —OVCF3 |
| —C≡N | —C |
| 3) Bonding group —Zₙ— | Symbol |
| —CₙH₂ₙ— | n |
| —COO— | E |
| —CH═CH— | V |
| —CH₂O— | 1O |
| —OCH₂— | O1 |
| —CF₂O— | X |
| —C≡C— | T |
| 4) Ring structure —Aₙ— | Symbol |
| 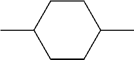 | H |
| 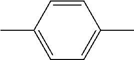 | B |
| 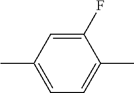 | B(F) |
| 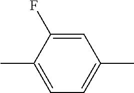 | B(2F) |
| 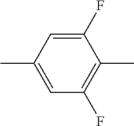 | B(F,F) |
| 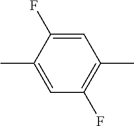 | B(2F,5F) |
| 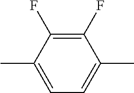 | B(2F,3F) |
| 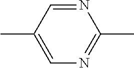 | Py |
| 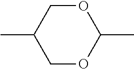 | G |
| 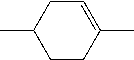 | ch |

5) Examples of description
Example 1 3—HB—CL

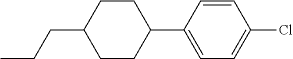 [SMILES: CCCC1CCC(c2ccc(Cl)cc2)CC1]

Example 2 5—HHBB(F,F)—F

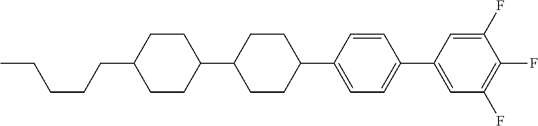 [SMILES: CCCCCC1CCC(C2CCC(c3ccc(-c4cc(F)c(F)c(F)c4)cc3)CC2)CC1]

Example 3 3—HB—O2

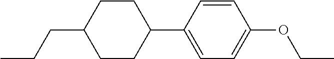 [SMILES: CCCC1CCC(c2ccc(OCC)cc2)CC1]

Example 4 3—HBB(F,F)—F

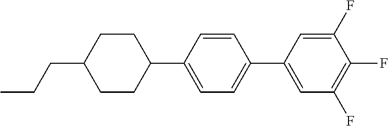 [SMILES: CCCC1CCC(c2ccc(-c3cc(F)c(F)c(F)c3)cc2)CC1]

### Use Example 1

| | | |
|---|---|---|
| 2-HB—C | (8-1) | 7% |
| 3-HB—C | (8-1) | 10% |
| 3-HB—O2 | (2-5) | 11% |
| 2-BTB-1 | (2-10) | 5% |
| 3-HHB—F | (6-1) | 5% |
| 3-HHB-1 | (3-1) | 9% |
| 3-HHB—O1 | (3-1) | 6% |
| 3-HHB-3 | (3-1) | 15% |
| 3-HHEB—F | (6-10) | 3% |
| 5-HHEB—F | (6-10) | 4% |
| 2-HHB(F)—F | (6-2) | 7% |
| 3-HHB(F)—F | (6-2) | 6% |
| 5-HHB(F)—F | (6-2) | 6% |
| 3-HHB(F, F)—F | (6-3) | 6% |

Compound (No. 265) described below was added to the composition described above in a proportion of 0.3% by weight.

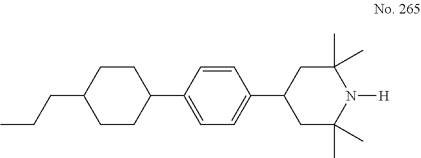 [SMILES: CCCC1CCC(c2ccc(C3CC(C)(C)NC(C)(C)C3)cc2)CC1]

NI=101.6° C.; η=18.0 mPa·s; Δn=0.103; Δε=4.6.

### Use Example 2

| | | |
|---|---|---|
| 3-HB—CL | (5-2) | 13% |
| 3-HH-4 | (2-1) | 15% |
| 3-HB—O2 | (2-5) | 8% |
| 3-HHB(F, F)—F | (6-3) | 6% |
| 3-HBB(F, F)—F | (6-24) | 25% |
| 5-HBB(F, F)—F | (6-24) | 23% |
| 5-HBB(F)B-2 | (4-5) | 5% |
| 5-HBB(F)B-3 | (4-5) | 5% |

Compound (No. 277) described below was added to the composition described above in a proportion of 0.2% by weight.

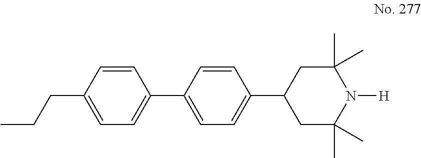 [SMILES: CCCc1ccc(-c2ccc(C3CC(C)(C)NC(C)(C)C3)cc2)cc1]

NI=71.5° C.; η=17.2 mPa·s; Δn=0.112; Δε=5.0.

### Use Example 3

| | | |
|---|---|---|
| 7-HB(F, F)—F | (5-4) | 3% |
| 3-HB-O2 | (2-5) | 7% |
| 2-HHB(F)—F | (6-2) | 10% |
| 3-HHB(F)—F | (6-2) | 10% |
| 5-HHB(F)—F | (6-2) | 8% |
| 2-HBB(F)—F | (6-23) | 9% |
| 3-HBB(F)—F | (6-23) | 11% |
| 5-HBB(F)—F | (6-23) | 15% |
| 2-HBB—F | (6-22) | 4% |
| 3-HBB—F | (6-22) | 4% |
| 5-HBB—F | (6-22) | 4% |
| 3-HBB(F, F)—F | (6-24) | 4% |
| 5-HBB(F, F)—F | (6-24) | 11% |

Compound (No. 197) described below was added to the composition described above in a proportion of 0.05% by weight.

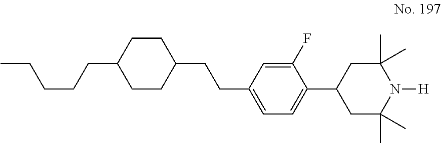 [SMILES: CCCCCC1CCC(CCc2ccc(C3CC(C)(C)NC(C)(C)C3)c(F)c2)CC1]

NI=85.1° C.; η=25.2 mPa·s; Δn=0.116; Δε=5.8.

### Use Example 4

| | | |
|---|---|---|
| 5-HB—CL | (5-2) | 18% |
| 3-HH-4 | (2-1) | 11% |
| 3-HH-5 | (2-1) | 3% |
| 3-HHB—F | (6-1) | 4% |
| 3-HHB—CL | (6-1) | 4% |
| 4-HHB—CL | (6-1) | 4% |
| 3-HHB(F)—F | (6-2) | 11% |
| 4-HHB(F)—F | (6-2) | 9% |
| 5-HHB(F)—F | (6-2) | 8% |
| 7-HHB(F)—F | (6-2) | 8% |
| 5-HBB(F)—F | (6-23) | 3% |
| 1O1-HBBH-5 | (4-1) | 3% |
| 3-HHBB(F, F)—F | (7-6) | 2% |
| 4-HHBB(F, F)—F | (7-6) | 3% |
| 5-HHBB(F, F)—F | (7-6) | 3% |
| 3-HH2BB(F, F)—F | (7-15) | 3% |
| 4-HH2BB(F, F)—F | (7-15) | 3% |

Compound (No. 198) described below was added to the composition described above in a proportion of 0.005% by weight.

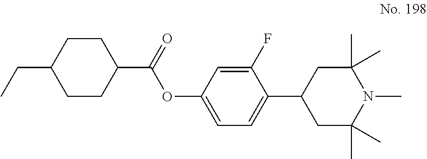 [SMILES: CCC1CCC(C(=O)Oc2ccc(C3CC(C)(C)N(C)C(C)(C)C3)c(F)c2)CC1]

NI=113.2° C.; η=19.0 mPa·s; Δn=0.092; Δε=3.9.

### Use Example 5

| | | |
|---|---|---|
| 3-HHB(F, F)—F | (6-3) | 7% |
| 3-H2HB(F, F)—F | (6-15) | 7% |
| 4-H2HB(F,F)—F | (6-15) | 7% |
| 5-H2HB(F, F)—F | (6-15) | 9% |
| 3-HBB(F, F)—F | (6-24) | 21% |
| 5-HBB(F, F)—F | (6-24) | 21% |
| 3-H2BB(F, F)—F | (6-27) | 11% |
| 5-HHBB(F, F)—F | (7-6) | 4% |
| 5-HHEBB—F | (7-17) | 2% |
| 3-HH2BB(F, F)—F | (7-15) | 4% |
| 1O1-HBBH—4 | (4-1) | 4% |
| 1O1-HBBH—5 | (4-1) | 3% |

Compound (No. 206) described below was added to the composition described above in a proportion of 0.06% by weight.

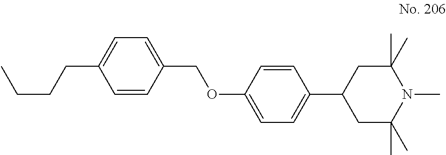 [SMILES: CCCCc1ccc(COc2ccc(C3CC(C)(C)N(C)C(C)(C)C3)cc2)cc1]

NI=98.3° C.; η=35.7 mPa·s; Δn=0.118; Δε=9.1.

### Use Example 6

| | | |
|---|---|---|
| 5-HB—F | (5-2) | 12% |
| 6-HB—F | (5-2) | 9% |
| 7-HB—F | (5-2) | 7% |
| 2-HHB—OCF3 | (6-1) | 9% |
| 3-HHB—OCF3 | (6-1) | 6% |
| 4-HHB—OCF3 | (6-1) | 7% |
| 5-HHB—OCF3 | (6-1) | 4% |
| 3-HH2B—OCF3 | (6-4) | 4% |
| 5-HH2B—OCF3 | (6-4) | 4% |
| 3-HHB(F, F)—OCF2H | (6-3) | 3% |
| 3-HHB(F, F)—OCF3 | (6-3) | 3% |
| 3-HH2B(F)—F | (6-5) | 3% |
| 3-HBB(F)—F | (6-23) | 10% |
| 5-HBB(F)—F | (6-23) | 10% |
| 5-HBBH-3 | (4-1) | 6% |
| 3-HB(F)BH-3 | (4-2) | 3% |

Compound (No. 208) described below was added to the composition described above in a proportion of 0.01% by weight.

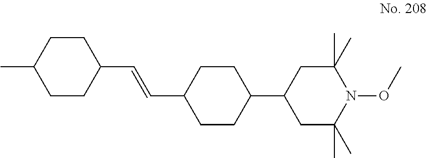

NI=89.9° C.; η=15.2 mPa·s; Δn=0.094; Δε=4.2.

### Use Example 7

| | | |
|---|---|---|
| 5-HB—CL | (5-2) | 14% |
| 3-HH-4 | (2-1) | 7% |
| 3-HHB-1 | (3-1) | 4% |
| 3-HHB(F, F)—F | (6-3) | 8% |
| 3-HBB(F, F)—F | (6-24) | 18% |
| 5-HBB(F, F)—F | (6-24) | 16% |
| 3-HHEB(F, F)—F | (6-12) | 10% |
| 4-HHEB(F, F)—F | (6-12) | 4% |
| 5-HHEB(F, F)—F | (6-12) | 3% |
| 2-HBEB(F, F)—F | (6-39) | 4% |
| 3-HBEB(F, F)—F | (6-39) | 4% |
| 5-HBEB(F, F)—F | (6-39) | 3% |
| 3-HHBB(F, F)—F | (7-6) | 5% |

Compound (No. 289) described below was added to the composition described above in a proportion of 0.1% by weight.

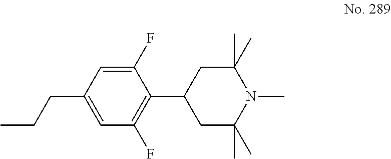 [SMILES: CCCc1cc(F)c(C2CC(C)(C)N(C)C(C)(C)C2)c(F)c1]

NI=76.3° C.; η=21.5 mPa·s; Δn=0.102; Δε=8.7.

### Use Example 8

| | | |
|---|---|---|
| 3-HB—CL | (5-2) | 4% |
| 5-HB—CL | (5-2) | 4% |
| 3-HHB—OCF3 | (6-1) | 6% |
| 3-H2HB—OCF3 | (6-13) | 5% |
| 5-H4HB—OCF3 | (6-19) | 15% |
| V-HHB(F)—F | (6-2) | 4% |
| 3-HHB(F)—F | (6-2) | 4% |
| 5-HHB(F)—F | (6-2) | 6% |
| 3-H4HB(F, F)—CF3 | (6-21) | 8% |
| 5-H4HB(F, F)—CF3 | (6-21) | 10% |
| 5-H2HB(F, F)—F | (6-15) | 6% |
| 5-H4HB(F, F)—F | (6-21) | 7% |
| 2-H2BB(F)—F | (6-26) | 5% |
| 3-H2BB(F)—F | (6-26) | 12% |
| 3-HBEB(F, F)—F | (6-39) | 4% |

Compound (No. 265) described below was added to the composition described above in a proportion of 0.08% by weight.

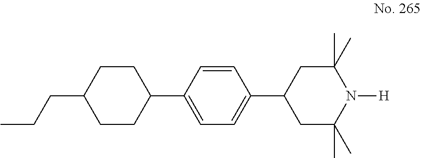 [SMILES: CCCC1CCC(c2ccc(C3CC(C)(C)NC(C)(C)C3)cc2)CC1]

NI=72.1° C.; η=25.9 mPa·s; Δn=0.098; Δε=8.2.

### Use Example 9

| | | |
|---|---|---|
| 5-HB—CL | (5-2) | 14% |
| 7-HB(F,F)—F | (5-4) | 4% |
| 3-HH-4 | (2-1) | 12% |
| 3-HH-5 | (2-1) | 6% |
| 3-HB—O2 | (2-5) | 14% |
| 3-HHB-1 | (3-1) | 7% |
| 3-HHB—O1 | (3-1) | 5% |
| 2-HHB(F)—F | (6-2) | 8% |
| 3-HHB(F)—F | (6-2) | 6% |
| 5-HHB(F)—F | (6-2) | 7% |
| 3-HHB(F, F)—F | (6-3) | 8% |
| 3-H2HB(F, F)—F | (6-15) | 5% |
| 4-H2HB(F, F)—F | (6-15) | 4% |

Compound (No. 277) described below was added to the composition described above in a proportion of 0.0051 by weight.

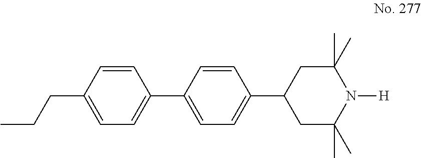 [SMILES: CCCc1ccc(-c2ccc(C3CC(C)(C)NC(C)(C)C3)cc2)cc1]

NI=71.1° C.; η=13.6 mPa·s; Δn=0.072; Δε=2.8.

### Use Example 10

| | | |
|---|---|---|
| 5-HB—CL | (5-2) | 5% |
| 7-HB(F)—F | (5-3) | 5% |
| 3-HH-4 | (2-1) | 8% |
| 3-HH-5 | (2-1) | 12% |
| 3-HB—O2 | (2-5) | 11% |
| 3-HHEB—F | (6-10) | 10% |
| 5-HHEB—F | (6-10) | 8% |
| 3-HHEB(F, F)—F | (6-12) | 10% |
| 4-HHEB(F, F)—F | (6-12) | 6% |
| 3-GHB(F, F)—F | (6-109) | 4% |
| 4-GHB(F, F)—F | (6-109) | 5% |
| 5-GHB(F, F)—F | (6-109) | 6% |
| 2-HHB(F, F)—F | (6-3) | 5% |
| 3-HHB(F, F)—F | (6-3) | 5% |

Compound (No. 197) described below was added to the composition described above in a proportion of 0.031 by weight.

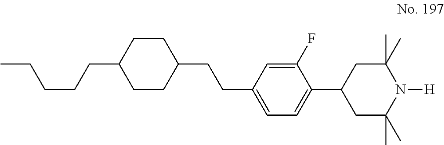 [SMILES: CCCCCC1CCC(CCc2ccc(C3CC(C)(C)NC(C)(C)C3)c(F)c2)CC1]

NI=75.8° C.; η=18.3 mPa·s; Δn=0.069; Δε=5.5.

### Use Example 11

| | | |
|---|---|---|
| 1V2-BEB(F, F)—C | (8-15) | 4% |
| 3-HB—C | (8-1) | 19% |
| 2-BTB-1 | (2-10) | 11% |
| 5-HH—VFF | (2-1) | 27% |
| 3-HHB-1 | (3-1) | 5% |
| VFF—HHB-1 | (3-1) | 8% |
| VFF2-HHB-1 | (3-1) | 11% |
| 3-H2BTB-2 | (3-17) | 5% |
| 3-H2BTB-3 | (3-17) | 5% |
| 3-H2BTB-4 | (3-17) | 5% |

Compound (No. 198) described below was added to the composition described above in a proportion of 0.02% by weight.

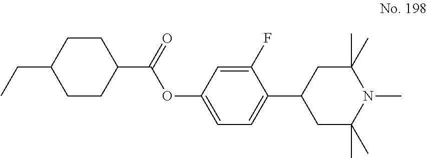 [SMILES: CCC1CCC(C(=O)Oc2ccc(C3CC(C)(C)N(C)C(C)(C)C3)c(F)c2)CC1]

NI=85.2° C.; η=11.6 mPa·s; Δn=0.135; Δε=5.3.

### Example 1

| | | |
|---|---|---|
| 3-BB(2F, 3F)—O2 | (2-4) | 13% |
| 2-HH1OB(2F, 3F)—O2 | (2-8) | 20% |
| 3-HH1OB(2F, 3F)O—2 | (2-8) | 14% |
| 3-HH—V | (3-1) | 31% |
| 1-BB-5 | (3-3) | 8% |
| 3-HHB-1 | (3-5) | 4% |
| 3-HHB-3 | (3-5) | 3% |
| 5-B(F)BB-2 | (3-8) | 7% |

### Comparative Example 1

Comparative compound (S-7), compound 265 and compound 277 were added to the composition described in Example 1 in a proportion of 0.10% by weight. Line image sticking parameters (LISP) were 11.3%, 1.5% and 3.4%, respectively (Table 4). Compound (265) and compound (277) are found to be superior to the comparative compound from the result.

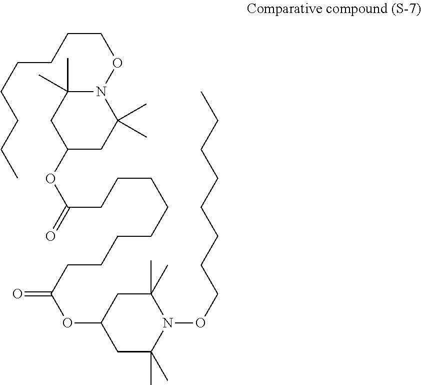 [SMILES: CCCCCCCCON1C(C)(C)CC(OC(=O)CCCCCCCCC(=O)OC2CC(C)(C)N(OCCCCCCCC)C(C)(C)C2)CC1(C)C]

TABLE 4
Comparison of line image sticking parameter

| | | | Line image |
|---|---|---|---|
| | | Amount of | sticking |
| | Additive | addition | parameter |
| Example 1 | Compound 265 | 0.10% by weight | 1.5% |
| Example 2 | Compound 277 | 0.10% by weight | 3.4% |
| Comparative | Comparative | 0.10% by weight | 11.3% |
| Example 1 | compound | | |

### INDUSTRIAL APPLICABILITY

A compound of the invention is useful as a light stabilizer. A liquid crystal composition containing the compound can be widely utilized to a liquid crystal display device used in a computer monitor, a television and so forth.

What is claimed is:

1. A compound, represented by formula (1): 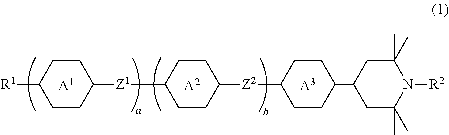
wherein, in formula (1),
R¹ is alkyl having 1 to 10 carbons, alkoxy having 1 to 10 carbons or alkenyl having 2 to 10 carbons, and in the groups, at least one hydrogen may be replaced by fluorine or chlorine;
R² is hydrogen, hydroxy, oxy radical, alkyl having 1 to 10 carbons, alkoxy having 1 to 10 carbons or alkenyl having 2 to 10 carbons, and in the groups, at least one hydrogen bound to carbon may be replaced by fluorine or chlorine;
ring A¹, ring A² and ring A³ are independently 1,4-cyclohexylene, 1,4-cyclohexenylene, 3,4-dihydro-2H-pyrane-2,5-diyl, 3,4-dihydro-2H-pyrane-3,6-diyl, 3,6-dihydro-2H-pyrane-2,5-diyl, tetrahydropyran-2,5-diyl, 1,3-dioxane-2,5-diyl, 1,4-phenylene, pyridine-2,5-diyl, decahydronaphthalene-2,6-diyl, naphthalene-1,3-diyl, naphthalene-1,4-diyl, naphthalene-1,5-diyl, naphthalene-1,6-diyl, naphthalene-1,7-diyl, naphthalene-1,8-diyl, naphthalene-2,3-diyl, naphthalene-2,6-diyl or naphthalene-2,7-diyl, and in the rings, at least one hydrogen on an aromatic ring may be replaced by fluorine, chlorine, cyano, alkyl having 1 to 5 carbons, alkoxy having 1 to 5 carbons, alkyl having 1 to 5 carbons in which at least one hydrogen is replaced by fluorine or chlorine, or alkoxy having 1 to 5 carbons in which at least one hydrogen is replaced by fluorine or chlorine;
Z¹ and Z² are independently a single bond or alkylene having 1 to 10 carbons, and in the alkylene, at least one piece of —CH₂— may be replaced by —O—, —COO— or —OCO—, and at least one piece of —CH₂—CH₂— may be replaced by —CH═CH— or —C≡C—, and in the groups, at least one hydrogen may be replaced by fluorine or chlorine; and
a and b are independently 0 or 1.

2. The compound according to claim 1, wherein, in formula (1), R¹ is alkyl having 1 to 10 carbons, alkoxy having 1 to 10 carbons or alkenyl having 2 to 10 carbons; R² is hydrogen, hydroxy, oxy radical, alkyl having 1 to 10 carbons, alkoxy having 1 to 10 carbons or alkenyl having 2 to 10 carbons; ring A¹, ring A² and ring A³ are independently 1,4-cyclohexylene, 1,4-cyclohexenylene, tetrahydropyran-2,5-diyl, 1,3-dioxane-2,5-diyl, 1,4-phenylene, pyridine-2,5-diyl or naphthalene-2,6-diyl, and in rings, at least one hydrogen on an aromatic ring may be replaced by fluorine, chlorine, —CF₃, —CHF₂, —CH₂F, —OCF₃, —OCHF₂ or —OCH₂F; Z¹ and Z² are independently a single bond, —COO—, —OCO—, —CH₂O—, —OCH₂—, —CF₂O—, —OCF₂—, —CH₂CH₂—, —CF₂CF₂—, —CH═CH—, —CF═CF—, —CH₂CH₂CH₂CH₂— or —CH₂CH═CHCH₂—; and a and b are independently 0 or 1.

3. The compound according to claim 1, wherein, in formula (1), R¹ is alkyl having 1 to 10 carbons, alkoxy having 1 to 10 carbons or alkenyl having 2 to 10 carbons; R² is hydrogen, hydroxy, oxy radical, alkyl having 1 to 10 carbons, alkoxy having 1 to 10 carbons or alkenyl having 2 to 10 carbons; ring A¹, ring A² and ring A³ are independently 1,4-cyclohexylene, 1,4-cyclohexenylene, tetrahydropyran-2,5-diyl, 1,3-dioxane-2,5-diyl, 1,4-phenylene or pyridine-2,5-diyl, and in the rings, at least one hydrogen on an aromatic ring may be replaced by fluorine or chlorine; Z¹ and Z² are independently a single bond, —COO—, —CH₂O—, —CF₂O—, —CH₂CH₂— or —CH═CH—; and a and b are independently 0 or 1.

4. The compound according to claim 1, wherein, in formula (1), R¹ is alkyl having 1 to 8 carbons, alkenyl having 2 to 8 carbons or alkoxy having 2 to 8 carbons; R² is hydrogen, hydroxy, oxy radical, alkyl having 1 to 8 carbons, alkoxy having 1 to 8 carbons or alkenyl having 2 to 8 carbons; ring A¹, ring A² and ring A³ are independently 1,4-cyclohexylene, 1,4-phenylene, or 1,4-phenylene in which at least one hydrogen is replaced by fluorine; Z¹ and Z² are independently a single bond, —COO—, —CH₂O— or —CH₂CH₂—; and a and b are independently 0 or 1.

5. The compound according to claim 1, represented by formula (1a) or (1b): 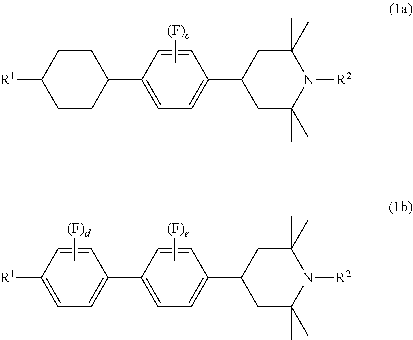
wherein, in formula (1a) or (1b), R¹ is straight-chain alkyl having 1 to 8 carbons, straight-chain alkoxy having 2 to 8 carbons or straight-chain alkenyl having 2 to 8 carbons; R² is hydrogen, hydroxy, oxy radical, straight-chain alkyl having 1 to 8 carbons, straight-chain alkoxy having 1 to 8 carbons or straight-chain alkenyl having 2 to 8 carbons; and c, d and e are independently an integer from 0 to 4.

6. The compound according to claim 5, wherein, in formula (1a) or (1b), R¹ is straight-chain alkyl having 1 to 8 carbons; R² is hydrogen, hydroxy, oxy radical or straight-chain alkyl having 1 to 8 carbons; and c, d and e are independently 0 or 1.

7. The compound according to claim 1, represented by formula (1c) or (1d): 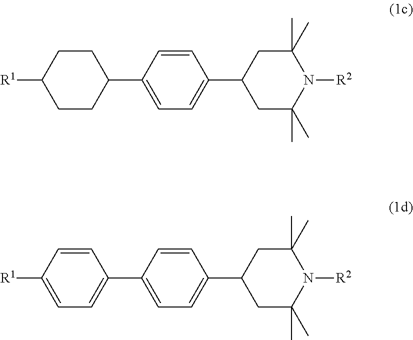
wherein, in formula (1c) or (1d), R¹ is straight-chain alkyl having 1 to 6 carbons, straight-chain alkoxy having 1 to 6 carbons or straight-chain alkenyl having 2 to 6 carbons; and R² is hydrogen, hydroxy, oxy radical, straight-chain alkyl having 1 to 6 carbons, straight-chain alkoxy having 1 to 6 carbons or straight-chain alkenyl having 2 to 6 carbons.

8. The compound according to claim 7, wherein, in formula (1c) or (1d), R¹ is straight-chain alkyl having 1 to 6 carbons; and R² is hydrogen, hydroxy, oxy radical or straight-chain alkyl having 1 to 6 carbons.

9. A liquid crystal composition, containing at least one compound according to claim 1.

10. The liquid crystal composition according to claim 9, further containing at least one compound selected from the group of compounds represented by formulas (2) to (4): 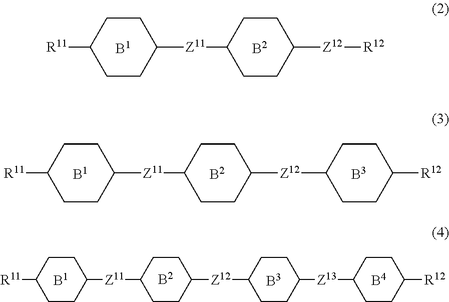
wherein, in formulas (2) to (4),
R¹¹ and R¹² are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine;
ring B¹, ring B², ring B³ and ring B⁴ are independently 1,4-cyclohexylene, 1,4-phenylene, 2-fluoro-1,4-phenylene, 2,5-difluoro-1,4-phenylene or pyrimidine-2,5-diyl; and
Z¹¹, Z¹² and Z¹³ are independently a single bond, —COO—, —CH₂CH₂—, —CH═CH— or —C≡C—.

11. The liquid crystal composition according to claim 9, further containing at least one compound selected from the group of compounds represented by formulas (5) to (7): 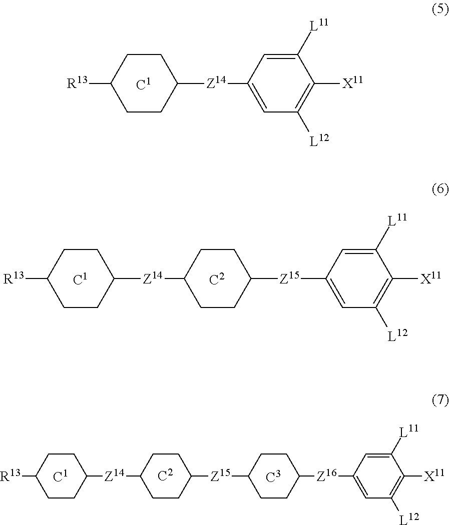
wherein, in formulas (5) to (7),
R¹³ is alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine;
X¹¹ is fluorine, chlorine, —OCF₃, —OCHF₂, —CF₃, —CHF₂, —CH₂F, —OCF₂CHF₂ or —OCF₂CHFCF₃;
ring C¹, ring C² and ring C³ are independently 1,4-cyclohexylene, 1,4-phenylene, 1,4-phenylene in which at least one hydrogen is replaced by fluorine, tetrahydropyran-2,5-diyl, 1,3-dioxane-2,5-diyl, or pyrimidine-2,5-diyl;
Z¹⁴, Z¹⁵ and Z¹⁶ are independently a single bond, —COO—, —OCO—, —CH₂O—, —OCH₂—, —CF₂O—, —OCF₂—, —CH₂CH₂—, —CH═CH—, —C≡C— or —(CH₂)₄—; and
L¹¹ and L¹² are independently hydrogen or fluorine.

12. The liquid crystal composition according to claim 9, further containing at least one compound selected from the group of compounds represented by formula (8): 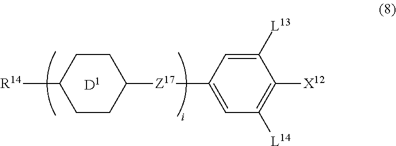
wherein, in formula (8),
R¹⁴ is alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine;
X¹² is —C≡N or —C≡C—C≡N;
ring D¹ is independently 1,4-cyclohexylene, 1,4-phenylene, 1,4-phenylene in which at least one hydrogen is replaced by fluorine, tetrahydropyran-2,5-diyl, 1,3-dioxane-2,5-diyl, or pyrimidine-2,5-diyl;
Z¹⁷ is independently a single bond, —COO—, —OCO—, —CH₂O—, —OCH₂—, —CF₂O—, —OCF₂—, —CH₂CH₂— or —C≡C—;
L¹³ and L¹⁴ are independently hydrogen or fluorine; and
i is 1, 2, 3 or 4.

13. The liquid crystal composition according to claim 9, further containing at least one compound selected from the group of compounds represented by formulas (9) to (15): 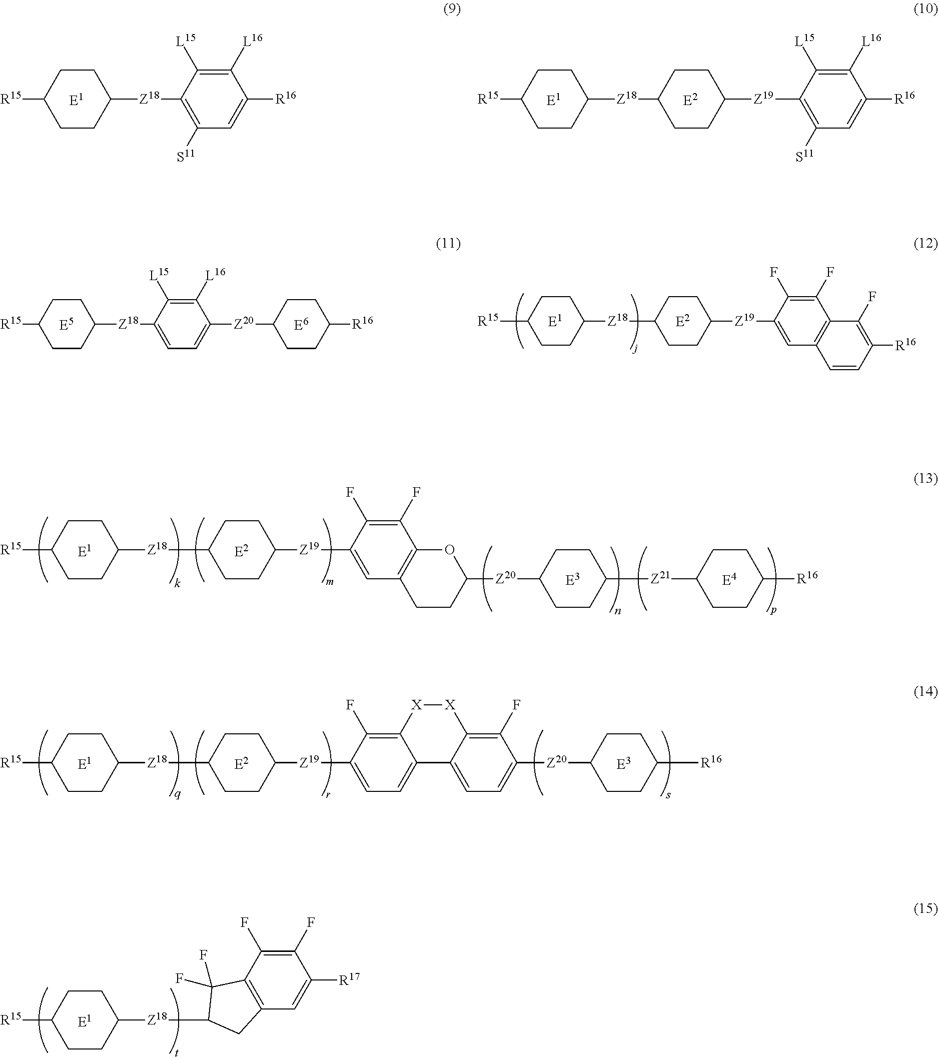
wherein, in formulas (9) to (15),
R¹⁵, R¹⁶ and R¹⁷ are independently alkyl having 1 to 10 carbons or alkenyl having 2 to 10 carbons, and in the alkyl and the alkenyl, at least one piece of —CH₂— may be replaced by —O—, and in the groups, at least one hydrogen may be replaced by fluorine, and R¹⁷ may be hydrogen or fluorine;
ring E¹, ring E², ring E³ and ring E⁴ are independently 1,4-cyclohexylene, 1,4-cyclohexenylene, 1,4-phenylene, 1,4-phenylene in which at least one hydrogen is replaced by fluorine, tetrahydropyran-2,5-diyl or decahydronaphthalene-2,6-diyl;
ring E⁵ and ring E⁶ are independently 1,4-cyclohexylene, 1,4-cyclohexenylene, 1,4-phenylene, tetrahydropyran-2,5-diyl or decahydronaphthalene-2,6-diyl;
Z¹⁸, Z¹⁹, Z²⁰ and Z²¹ are independently a single bond, —COO—, —OCO—, —CH₂O—, —OCH₂—, —CF₂O—, —OCF₂—, —CH₂CH₂—, —CF₂OCH₂CH₂— or —OCF₂CH₂CH₂—;
L¹⁵ and L¹⁶ are independently fluorine or chlorine;
S¹¹ is hydrogen or methyl;
X is —CHF— or —CF₂—; and
j, k, m, n, p, q, r and s are independently 0 or 1, a sum of k, m, n and p is 1 or 2, a sum of q, r and s is 0, 1, 2 or 3, and t is 1, 2 or 3.

14. A liquid crystal display device, including at least one liquid crystal composition according to claim 9.
